(19) United States
(12) Patent Application Publication
(10) Pub. No.: US 2009/0111837 A1
(43) Pub. Date: Apr. 30, 2009

(54) USE OF PDE7 INHIBITORS FOR THE TREATMENT OF NEUROPATHIC PAIN

(76) Inventors: Peter Cox, Kent (GB); Ross Anderson Kinloch, Kent (GB); Graham Nigel Maw, Kent (GB)

Correspondence Address: PFIZER INC., PATENT DEPARTMENT, MS8260-1611, EASTERN POINT ROAD, GROTON, CT, 06340, US

(21) Appl. No.: 11/817,528

(22) PCT Filed: Feb. 16, 2006
(86) PCT No.: PCT/IB06/00369

§ 371 Date: Nov. 6, 2008

Related U.S. Application Data
(60) Provisional application No. 60/675,761, filed on Apr. 27, 2005.

(30) Foreign Application Priority Data
Mar. 1, 2005 (GB) 0504209.8

(51) Int. Cl. A61K 31/527; C07D 239/72; A61P 25/02; A61P 25/00

(52) U.S. Cl. 514/267; 544/231

(57) ABSTRACT

The present invention relates to the use of a phosphodiesterase 7 (PDE7) inhibitor in the manufacture of a medicament for the treatment of neuropathic pain and to a method of treating neuropathic pain using an inhibitor of PDE7.

### FIELD OF THE INVENTION

The invention relates to the use of a phosphodiesterase 7 (PDE7) inhibitor in the manufacture of a medicament for the treatment of neuropathic pain and to a method of treating neuropathic pain using an inhibitor of PDE7.

### BACKGROUND OF THE INVENTION

Phosphodiesterases (PDEs) are a family of enzymes which affect various cellular signaling processes by the process of hydrolyzing the second messenger molecules cAMP and cGMP to the corresponding inactive 5′-monophosphate nucleotides and thereby regulating their physiological level. The secondary messengers cAMP and cGMP are responsible for the regulation of numerous intracellular processes. There are at least 11 families of PDE's, some (PDE3, 4, 7, 8) being specific for cAMP, and others for cGMP (PDE5, 6, and 9).

PDE7 is one member of the PDE family and comprises 2 subclass members PDE7 A and B. The mRNA of PDE7 is expressed in various tissues and cell types known to be important in the pathogenesis of several diseases such as Tcell related disorders, in particular PDE7A and its splice variants are upregulated in activated Tcells, [L. Li, C. Yee and J. A. Beavo. Science 283 (1999), pp. 848-851], and in B-lymphocytes. [R. Lee, S. Wolda, E. Moon, J. Esselstyn, C. Hertel and A. Lerner. Cell. Signal 14 (2002), pp. 277-284], autoimmune disease. [L. Li, C. Yee and J. A. Beavo. Science 283 (1999), pp. 848-851], and airway disease [Smith S J, et al Am. J. Physiol. Lung. Cell. Mol. Physiol. 2003, 284, L279-L289]. Consequently it is expected that selective inhibitors of PDE7 will have broad application as both immunosuppressants and treatment for respiratory conditions, for example chronic obstructive pulmonary disease and asthma. [N. A. Glavas, C. Ostenson, J. B. Schaefer, V. Vasta and J. A. Beavo. PNAS 98 (2001), pp. 6319-6324.]

Studies in rat have shown that PDE7A mRNA is found to be widely distributed in rat brain in both neuronal and non-neuronal cell populations. The highest levels are observed in the olfactory bulb, olfactory tubercle, hippocampus, cerebellum, medial habenula nucleus, pineal gland, area postrema, and choroid plexus. PDE7A mRNA is also widely detected in other non brain tissue. These results are consistent with PDE7A being involved in the regulation of cAMP signaling in many brain functions and suggests that PDE7A could have an effect on memory, depression, and emesis [X. Miró, S. Pérez-Torres, J. M. Palacios, P. Puigdomènech, G. Mengod Synapse 40:201-214, 2001] a link to Alzheimers disease is also suggested [S. Pérez Torres R, Cortés M, Tolnay A., Probst J. M., Palaciosand G. Mengod, Experimental Neurology, 182,2, August 2003, Pages 322-334]. Additionally PDE7 has also been implicated in both fertility disorders [WO 0183772] and leukemia [Lee r, et. al. Cell Signalling 2002, 14, 277-284]. PDE7A has been isolated from yeast [Michaeli, T., et al J. Biol. Chem. 268 1993 12925-12932], human [Han, P., Xiaoyan, Z., Tamar, M., Journ. Biol. Chem. 272 26 1997 16152-16157], mouse [Bloom, T., Beavo, J A., proc. Natl. Acad. Sci. USA 93 1996 14188-14192] and mouse, and upregulation of PDE7A levels is seen in human T lymphocytes [Ichimura, M., Kase, H. Biochem. Biophys. Res. Commun 193, 1993 985-990]. PDE7B, the second member of the PDE7 family, shares 70% amino acid homology with PDE7A in the C-terminal catalytic domain (N terminal domain is the regulatory domain containing the phosphorylation site which is conserved across the PDE family]. PDE7B is cAMP specific and has been cloned from mouse [accession number—AJ251858] and human [accession number—AJ251860] sources [C. Gardner, N. Robas, D. Cawkill and M. Fidock. Biochem. Biophys. Res. Commun. 272 (2000), pp. 186-192]. It has been shown to be expressed in a wide variety of tissues: the caudate nucleus, putamen and occipital lobe of the brain and peripherally in the heart, ovary and pituriary gland, kidney and liver small intestine and thymus, additionally in skeletal muscle, colon, bladder, uterus, prostate, stomach adrenal gland and thyroid gland. PDE7B has also been shown to discriminate among several general PDE inhibitors [J. M. Hetman, S. H. Soderling, N. A. Glavas and J. A. Beavo. PNAS 97 (2000), pp. 472-476], many standard PDE inhibitors, zaprinast, rolipram, milrinone do not specifically inhibit PDE7B.

The amino acid and nucleotide sequences that encode PDE7 of various species are known to those skilled in the art and can be found in GenBank under accession numbers AB057409, U77880, AB038040, L12052, AK035385, AY007702.

Inhibitors of PDE7 are known as is their use in the treatment of various PDE7 related diseases. The patent application EP1348701A1 (published: Jan. 10, 2003) discloses pharmaceutical compositions comprising phosphodiesterase 7 inhibitors. EP1348701A1 addresses the problem of providing a means of alleviating visceral pain using such compositions. Visceral pain is known to be a particular and narrow class of nociceptive pain. It is known that there are 2 fundamental and different types of pain: nociceptive pain and neuropathic pain. It is further known that nociceptive and neuropathic pain are clinically and mechanistically distinct from each other.

The clinical characteristics of nociceptive pain are determined by excessive and/or prolonged activation of specific sensory neurones A6 and C fibers. These may be activated by a mechanical, chemical, or thermal stimulus and become sensitised in chronic inflammatory conditions.

Neuropathic pain however is defined as pain which arises as a result of damage to or dysfunction of the nervous system. The clinical characteristics of neuropathic pain are therefore determined predominantly by the mechanisms, location, and severity of the neuropathologic process itself and arises from neurons that have themselves been damaged. Neuropathic pain has important elements which are mediated via activity in sensory nerves which do not normally convey pain, the Aβ neurones.

Additionally, in contrast to nociceptive pain, neuropathic pain is notoriously difficult to treat; it responds very poorly or not at all to standard analgesic therapies which are effective in the treatment of nociceptive pain such as nonsteroidal anti-inflammatory drugs and acetaminophen; and responds less predictably and less robustly to opioids than do nociceptive pain conditions. Effective treatments for nociceptive pain are not expected to extend to neuropathic pain. In addition, medicaments such as gabapentin, pregabalin and amitripiline, which provide some relief to neuropathic pain, are often not effective in the treatment of nociceptive pain. Thus for these reasons: difference in clinical characteristics, difference in mechanism and difference in amenability to treatment, neuropathic pain is clearly distinguished as different from nociceptive pain.

The present invention addresses the problem of the providing a new therapeutic use for PDE7 inhibitors and presents the suprising and advantageous finding that a pharmaceutical composition comprising phosphodiesterase 7 inhibitors as an active component is effective in the alleviation of neuropathic pain, the present application demonstrates the suprising technical effect of the compositions of the invention and their particularly advantageous analgesic effects for the treatment of neuropathic pain.

Neuropathic pain is a condition resulting from disease or trauma to peripheral nerves or the CNS. The International Association for the study of pain defines this condition as pain initiated or caused by a primary lesion or dysfunction in the nervous system. Thus this type of pain affects many patients with a wide range of ailments. Common causes include metabolic (e.g. painful diabetic neuropathy), trauma (e.g. phantom limb pain), infection (post-herpetic neuralgia & HIV) and nerve compression (e.g. cancer, back pain). It has been estimated that this condition affects approximately 1% of the population. Neuropathic pain patients often exhibit multiple pain symptoms including hyperlagesia (exaggerated pain to noxious stimulus), allodynia, (pain from a previously innocuous stimulus) as well as ongoing pain. Neuropathic pain is pathological as it has no protective role. It is often present well after the original cause has dissipated, commonly lasting for years significantly decreasing patients' quality of life (Woolf and Mannion 1999 Lancet 353: 1959-1964). Neuropathic pain is difficult to treat clinically due to the above mentioned multiple pain symptoms which may act via different pain pathways and are not always treatable by any one particular analgesic compound. It has previously been shown that many analgesic compounds, including opioids and non steroidal anti inflammatory drugs (NSAIDs) exhibit low levels or no analgesic efficacy for neuropathic pain.

Accordingly, there is a critical medical need to identify pharmaceutically active compounds that interfere with key steps of the neuropathic pain processes that contribute to these pain symptoms. Also there is a medical need to develop new combinations of analgesic compounds which in combination either act synergistically to avert neuropathic pain or in combination treat different symptoms of neuropathic pain.

Additionally it is advantageous to identify target enzymes involved in pain pathways which are centrally expressed in the central nervous system (CNS) and to identify pharmaceutically active compounds which exert an analgesic effect by acting centrally in the CNS and associated tissue. PDE7 has been shown to be centrally expressed in the CNS tissues including, but not necessarily restricted to the caudate nucleus, putamen and occipital lobe of the brain in humans as well as being expressed in a number of peripheral tissues too, [C. Gardner, N. Robas, D. Cawkill and M. Fidock. Biochem. Biophys. Res. Commun. 272 (2000), pp. 186-192].

PDE7 has been the target of inhibitor development as such inhibitors are considered to represent a path to the treatment of inflammatory and immunological disease particularly T-cell related disease. Several classes of inhibitors of PDE7 have been produced which present micromolar levels of binding affinity for example, benzyl derivatives of 2,1,3-benzo[3,2-a]thiadiazine 2,2-dioxides and 2,1,3-benzothieno[3,2-a]thiadiazine 2,2-dioxides [A. Castro, M. I. Abasolo, C. Gil, V. Segarra and A. Martinez. Eur. J. Med. Chem. 36 (2001), pp. 333-338]. Also known are a series of guanine analogues which have been assessed in vitro to have low micromolar inhibitor activity for PDE7 and to show selectivity over other PDE family members (the 8-bromo-9-substituted compounds being the most potent) Barnes Mj, Cooper N, Davenport R J, Biorg. Med. Chem. Lett. (2001) 23 (8): 1081-1083. Two related series of PDE7 inhibitors with sub-micromolar potency have been described in WO0198274 (CellTech Chiroscience Ltd). These are m-substituted phenyl-N-phenylsulfonamides particularly N-phenyl-3-benzoxazol-2-ylphenylsulfonamide and N-phenyl-3-benzimidazol-2-ylphenylsulfonamide derivatives, they represent a series of PDE7 inhibitors described as useful in the treatment of asthma and allergic diseases, via modulation of T cell function. A series of purine based inhibitors of PDE7 have been described [Pitts, W J., et al Biorg. Med. Chem. Lett 14 2004 2955-2958] which show good PDE7 selectivity and micromolar inhibitor activity. A further group of potent selective PDE7 inhibitors spiroquinazolinones [Iorthiois, E., et al Biorg. Med. Chem. Lett, 14 2004 4623-4626] and 5,8-disubstituted spirocyclohexane-quinazolinones particularly 5 substituted 8-chloro-spirocyclohexane-quinazolinones derivatives such as 5-alkoxy-8 chloro-quinazolinone [Bernardelli, P., et al Bioorg. Med. Chem. Lett, 14 2004 4627-4631] have been prepared and shown by in-vivo pharmacokinetic models to be effective selective PDE7 inhibitors. WO0174786 (Darwin Discovery Ltd) describes a series of heterobiarylsulphonamides, and also WO0068230 (Darwin Discovery Ltd) describes 9-(1,2,3,4-Tetrahydronapthalen-1-yl)-1,9-dihydropurin-6-one derivatives and their use as PDE7 inhibitors. Merck has produced a diverse selection of heterocyclic PDE7 inhibitors the details of which are presented in the following applications: imidazole derivatives—WO0129049 and WO0136425, isoxazole derivatives—WO0132175, pyrrole derivatives—WO0132618, imidazopyridine derivatives—WO0134601. A further group of PDE7 inhibitors are presented in [Vergne, F., et al Bioorg. Med. Chem. Left, 2004, 14, 4607-461] & [Vergne, F., et al Bioorg. Med. Chem. Lett, 2004, 14, 4615-4621] and comprise a group of thiadiazoles which demonstrate nanomolar selective PDE7 inhibitory activity.

### BRIEF DESCRIPTION OF THE INVENTION

The invention is directed to the use of a PDE7 inhibitor for the manufacture of a medicament for the treatment of neuropathic pain.

The present invention further provides a method of treatment for neuropathic pain, in a mammalian subject, which comprises administering to the subject a therapeutically effective amount of an inhibitor of PDE7.

### DETAILED DESCRIPTION OF THE INVENTION

In a preferred embodiment the PDE7 inhibitor is selected from those compounds generally or specifically disclosed in the published patent applications WO02/074754 (Warner Lambert), which discloses quinazolinones which are PDE7 inhibitors and are useful for the manufacture of a medicament for the treatment of neuropathic pain and for the treatment of neuropathic pain.

According to this embodiment the PDE7 inhibitor is a compound having the following formula (I), (II) or (III),

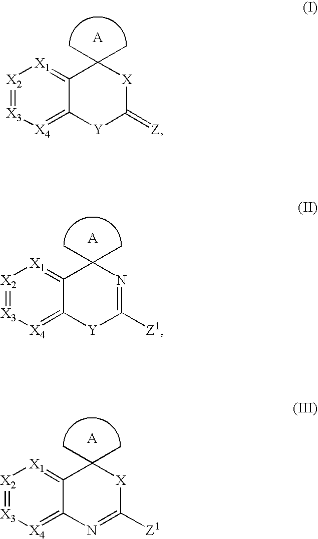

in which
a) X₁, X₂, X₃ and X₄ are the same or different and are selected from:
N, provided that not more than two of the groups X₁, X₂, X₃ and X₄ simultaneously represent a nitrogen atom, or,
C—R¹, in which R¹ is selected from:
   Q1, or
   lower alkyl, lower alkenyl or lower alkynyl, these groups being unsubstituted or substituted with one or several groups Q2;
   the group X⁵—R⁵ in which,
   X⁵ is selected from:
      a single bond,
      lower alkyl, lower alkenylene or lower alkynylene, optionally interrupted with 1 or 2 heteroatoms chosen from O, S, S(═O), SO₂ or N, the carbon atoms of these groups being unsubstituted or substituted with one or several groups, identical or different, selected from SR⁶, OR⁶, NR⁶R⁷, ═O, ═S or ═N—R⁶ in which R⁶ and R⁷ are the same or different and are selected from hydrogen or lower alkyl, and,
      R⁵ is selected from aryl, heteroaryl, cycloalkyl optionally interrupted with C(═O) or with 1, 2, or 3 heteroatoms chosen from O, S, S(═O), SO₂ or N, cycloalkenyl optionally interrupted with C(—O) or with 1, 2, or 3 heteroatoms chosen from O, S, S(═O), SO₂ or N, or a bicyclic group, these groups being unsubstituted or substituted with one or several groups selected from Q3, heteroaryl or lower alkyl optionally substituted with Q3;
in which Q1, Q2, Q3 are the same or different and are selected from
   hydrogen, halogen, CN, NO₂, SO₃H, P(═O)(OH)₂
   OR², OC(═O)R², C(═O)OR², SR², S(═O)R², NR³R⁴, Q-R², Q-NR³R⁴, NR²-Q-NR³R⁴ or NR³-Q-R² in which Q is selected from C(═NR), C(═O), C(═S) or SO₂, R is selected from hydrogen or lower alkyl and R², R³ and R⁴ are the same or different and are selected from:
      hydrogen,
      lower alkyl optionally interrupted with C(═O), (CH₂)ₙ-aryl, (CH₂)ₙ-heteroaryl, (CH₂)ₙ-cycloalkyl optionally interrupted with C(═O) or with 1 or 2 heteroatoms chosen from O, S, S(═O), SO₂ or N or (CH₂)ₙ-cycloalkenyl optionally interrupted with C(═O) or with 1 or 2 heteroatoms chosen from O, S, S(═O), SO₂ or N, in which n is an integer selected from 0, 1, 2, 3 or 4;
      these groups being unsubstituted or substituted with one or several groups selected from lower alkyl, halogen, CN, SO₃H, CH₃, SO₂CH₃, CF₃, C(═O)—NH—SO₂—CH₃, OR⁶, COOR⁶, NR⁶R⁷, C(═O)NR⁶R⁷ or SO₂NR⁶R⁷, in which R⁶ and R⁷ are the same or different and are selected from hydrogen or lower alkyl optionally substituted with one or two groups selected from OR, COOR or NRR⁸ in which R and R⁸ are hydrogen or lower alkyl, and,
      R⁶ and R⁷, and/or, R³ and R⁴, together with the nitrogen atom to which they are linked, can form a 4- to 8-membered heterocyclic ring, which may contain one or two heteroatoms selected from O, S, S(═O), SO₂ or N, and which may be substituted with,
         a 4- to 8-membered heterocyclic ring, which may contain one or two heteroatoms selected from O, S or N and which may be substituted with a lower alkyl, or,
         a lower alkyl optionally substituted with OR′, NR′R″, C(═O)NR′R″ or COOR′ in which R′ and R″ are the same or different and are selected from,
         H, or,
         lower alkyl optionally substituted with OR or COOR in which R is hydrogen or lower alkyl and,
      R′ and R″ together with the nitrogen atom to which they are linked, can form a 4- to 8-membered heterocyclic ring, which may contain one or two heteroatoms selected from O, S or N;
         b) X is O, S or NR⁹, in which R⁹ is selected from,
hydrogen, CN, OH, NH₂,
lower alkyl, lower alkenyl or lower alkynyl, these groups being unsubstituted or substituted with cycloalkyl optionally interrupted with 1 or 2 heteroatoms chosen from O, S, S(═O), SO₂ or N, cycloalkenyl optionally interrupted with 1 or 2 heteroatoms chosen from O, S, S(═O), SO₂ or N, aryl, heteroaryl, OR¹⁰ or NR¹⁰R¹¹ in which R¹⁰ and R¹¹ are the same or different and are selected from hydrogen or lower alkyl;
   c) Y is selected from O, S or N—R¹², in which R¹² is selected from:
hydrogen, CN, OH, NH₂,
lower alkyl, lower alkenyl or lower alkynyl, these groups being unsubstituted or substituted with, cycloalkyl optionally interrupted with 1 or 2 heteroatoms chosen from O, S, S(═O), SO₂ or N, cycloalkenyl optionally interrupted with 1 or 2 heteroatoms chosen from O, S, S(═O), SO₂ or N, aryl, heteroaryl, OR¹⁰ or NR¹⁰R¹¹ in which R¹⁰ and R¹¹ are the same or different and are selected from hydrogen or lower alkyl;
   d) Z is chosen from CH—NO₂, O, S or NR¹³ in which R¹³ is selected from:
hydrogen, CN, OH, NH₂, aryl, heteroaryl, cycloalkyl optionally interrupted with one or several heteroatoms chosen from O, S, S(═O), SO₂ or N, cycloalkenyl optionally interrupted with one or several heteroatoms chosen from O, S, S(═O), SO₂ or N, C(═O)R¹⁴, C(═O)NR¹⁴R¹⁵, OR¹⁴, or,
lower alkyl, unsubstituted or substituted with one or several groups which are the same or different and which are selected OR¹⁴ or NR¹⁴R¹⁵;
   R¹⁴ and R¹⁵ being independently selected from hydrogen or lower alkyl, or, R¹⁴ and R¹⁵, together with the nitrogen atom to which they are linked, can form a 4- to 8-membered heterocyclic ring which may contain one or two heteroatoms chosen from O, S or N, and which may be substituted with a lower alkyl;
   e) Z¹ is chosen from H, CH₃ or NR¹⁶R¹⁷ in which R¹⁶ and R¹⁷ are the same or different and are selected from:
hydrogen, CN, aryl, heteroaryl, cycloalkyl optionally interrupted with one or several heteroatoms chosen from O, S, S(═O), SO₂ or N, cycloalkenyl optionally interrupted with one or several heteroatoms chosen from O, S, S(═O), SO₂ or N, C(═O)R¹⁴, C(═O)NR¹⁴R¹⁵, OR¹⁴, or,
lower alkyl unsubstituted or substituted with one or several groups selected from OR¹⁴ or NR¹⁴R¹⁵,
   R¹⁴ and R¹⁵ being chosen from hydrogen or lower alkyl, and,
   R¹⁴ and R¹⁵, and/or, R¹⁶ and R¹⁷, together with the nitrogen atom to which they are linked, can form a 4- to 8-membered heterocyclic ring which may contain one or two heteroatoms chosen from O, S or N, and which may be substituted with a lower alkyl;
   f) A is a cycle chosen from:

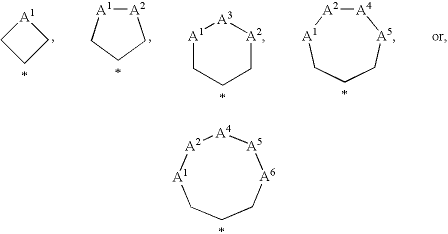

in which,
A¹, A², A³, A⁴, A⁵ and A⁶ are the same or different and are selected from O, S, C, C(═O), SO, SO₂ or N—R¹⁸ in which R¹⁸ is selected from:
   hydrogen, aryl, heteroaryl, cycloalkyl optionally interrupted with one or several heteroatoms chosen from O, S, S(═O), SO₂ or N, cycloalkenyl optionally interrupted with one or several heteroatoms chosen from O, S, S(═O), SO₂ or N,
   lower alkyl unsubstituted or substituted with aryl, heteroaryl, cycloalkyl optionally interrupted with one or several heteroatoms chosen from O, S, S(═O), SO₂ or N, cycloalkenyl optionally interrupted with one or several heteroatoms chosen from O, S, S(═O), SO₂ or N, CN, NR¹⁹R²⁰, C(═O)NR¹⁹R²⁰, OR¹⁹, C(═O)R¹⁹ or C(═O)OR¹⁹ in which R¹⁹ and R²⁰ are identical or different and are selected from hydrogen or lower alkyl;
* represents the carbon atom which is shared between the cycle A and the backbone cycle containing X and/or Y;
each carbon atom of the cycle A is unsubstituted or substituted with 1 or 2 groups, identical or different, selected from lower alkyl optionally substituted with OR²¹, NR²¹R²², COOR²¹ or CONR²¹R²², lower haloalkyl, CN, F, ═O, SO₂NR¹⁹R²⁰, OR¹⁹, SR¹⁹, C(═O)OR¹⁹, C(═O)NR¹⁹R²⁰ or NR¹⁹R²⁰ in which R¹⁹ and R²⁰ are identical or different and are selected from hydrogen or lower alkyl optionally substituted with OR²¹, NR²¹R²², COOR²¹ or CONR²¹R²² in which R²¹ and R²² identical or different and are selected from hydrogen or lower alkyl, and, R¹⁹ and R²⁰, and/or, R²¹ and R²², together with the nitrogen atom to which they are linked, can form a 4- to 8-membered heterocyclic ring;
2 atoms of the cycle A, which are not adjacent, may be linked by a 2, 3 or 4 carbon atom chain which may be interrupted with 1 heteroatom chosen from O, S or N;
   provided that not more than two of the groups A¹, A², A³, A⁴, A⁵ and A⁶ simultaneously represent a heteroatom;
   of their tautomeric forms, their racemic forms or their isomers and of their pharmaceutically acceptable derivatives, or a pharmaceutically acceptable salt or solvate thereof.

A particularly preferred PDE7 inhibitor disclosed in WO02/074754 is 5′-(3-(Carboxy)propoxy)-8′-chlorospiro[cyclohexane-1,4′-quinazolin]-2′(1′H)-one or a pharmaceutically acceptable salt or solvate thereof.

Alternatively the PDE7 inhibitor is an antibody, an antibody ligand binding domain or a polynucleotide.

Alternatively the PDE7 inhibitor is a compound of formula (IV) as disclosed in U.S. provisional patent application 60/741,854:

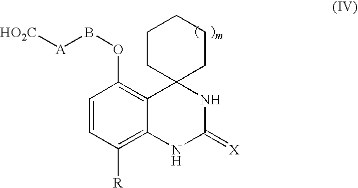

wherein:
m is 0, 1 or 2;

### X is O, S or N—CN;

### R is F, Cl or CN;

A is a C₃₋₆ cycloalkylene group optionally substituted with a C₁₋₄ alkyl group; and
B is a single bond or a C₁₋₂ alkylene group;
or a pharmaceutically acceptable salt, solvate or prodrug thereof.

Preferably in compounds of Formula (IV), m is 1 or 2, more preferably 1.

Preferably in compounds of Formula (IV), X is O or N—CN, more preferably O.

Preferably in compounds of Formula (IV), R is F or Cl, more preferably Cl.

Preferably in compounds of Formula (IV), A is a cyclobutylene or cyclohexylene group optionally substituted with a methyl group. More preferably, A is a cyclobutylene group. Even more preferably in compounds of formula IV, A is a 1,3-cyclobutylene group, especially a trans-1,3-cyclobutylene group.

Preferably in compounds of Formula (IV), B is a single bond or a methylene group. More preferably, B is a single bond.

Particularly preferred compounds of Formula (IV) include those in which each variable in Formula (IV) is selected from the suitable and/or preferred groups for each variable. Even more preferred compounds of Formula (IV) include those where each variable in Formula (IV) is selected from the more preferred or most preferred groups for each variable.

Alternatively the PDE7 inhibitor is a compound of formula (V) as disclosed in PCT published patent application WO04/026818:

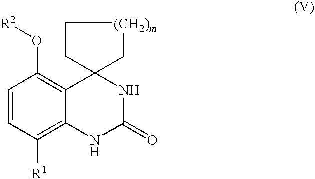

wherein,
m is 1, 2 or 3, and,
R¹ is selected from CH₃, Cl, Br and F and,
R² is selected from,
   Q¹-Q²-Q³-Q⁴ wherein,
      Q¹ is a single bond or a linear or branched (C₁-C₆)alkylene group;
      Q2 is a saturated 4 to 6-membered heterocycle comprising one or two heteroatoms selected from O or N;
      Q³ is a linear or branched (C₁-C₆)alkylene group;
      Q⁴ is a 4 to 8-membered, aromatic or non aromatic, heterocycle comprising 1 to 4 heteroatoms selected from O, S, S(═O), SO₂ and N, said heterocycle being optionally substituted with one or several groups selected from OR, NRR′, CN and (C₁-C₆)alkyl, wherein R and R′ are the same or different and are selected from H and (C₁-C₆)alkyl;
      the atom of Q² bound to Q¹ is a carbon atom, and,
      the atom of Q⁴ bound to Q³ is a carbon atom.
   (C₁-C₆)alkyl,
      said alkyl group being substituted with 1 to 3 groups, preferably 1, selected from OR⁴, COOR⁴, NR⁴R⁵, NRC(═O)R⁴, C(═O)NR⁴R⁵ and SO₂NR⁴R⁵, wherein,
         R is H or (C₁-C₆)alkyl;
         R⁴ is (C₁-C₆)alkyl substituted with one or several groups, preferably 1 to 3, selected from F, CN, S(═O)R⁶, SO₃H, SO₂R⁶, SR⁷, C(═O)—NH—SO₂—CH₃, C(═O)R⁷, NR′C(═O)R⁷, NR′SO₂R⁶, C(═O)NR⁷R⁸, O—C(═O)NR⁷R⁸ and SO₂NR⁷R⁸, wherein R′ is H or (C₁-C₆)alkyl, R⁶ is (C₁-C₆)alkyl optionally substituted with one or two groups OR″ wherein R″ is selected from H and (C₁-C₆)alkyl and R⁷ and R⁸ are the same or different and are selected from H and R⁶;
         R⁵ is selected from R⁴, H and (C₁-C₆)alkyl; or,
      said alkyl group being
      1) substituted with 1 to 3 groups, preferably 1, selected from OC(═O)R⁴, SR⁴, S(═O)R³, C(═NR⁹)R⁴, C(═NR⁹)—NR⁴R⁵, NR—C(—NR⁹)—NR⁴R⁵, NRCOOR⁴, NR—C(═O)—NR⁴R⁵, NR—SO₂—NR⁴R⁵, NR—C(═NR⁹)—R⁴ and NR—SO₂—R³ and,
      2) optionally substituted with 1 or 2 groups selected from OR⁴, COOR⁴, C(═O)—R⁴, NR⁴R⁵, NRC(═O)R⁴, C(═O)NR⁴R⁵ and SO₂NR⁴R⁵;
      wherein,
         R is selected from H and (C₁-C₆)alkyl;
         R⁹ is selected from H, CN, OH, OCH₃, SO₂CH₃, SO₂NH₂ and (C₁-C₆)alkyl, and,
         R³ is (C₁-C₆)alkyl, unsubstituted or substituted with one or several groups, preferably 1 to 3, selected from F, CN, S(═O)R⁶, SO₃H, SO₂R⁶, C(═O)—NH—SO₂—CH₃, OR⁷, SR⁷, COOR⁷, C(═O)R⁷, O—C(═O)NR⁷R⁸, NR⁷R⁸, NR′C(═O)R⁷, NR′SO₂R⁶, C(═O)NR⁷R⁸ and SO₂NR⁷R⁸, wherein R′ is H or (C₁-C₆)alkyl, R⁶ is (C₁-C₆)alkyl optionally substituted with one or two groups OR″, wherein R″ is selected from H and (C₁-C₆)alkyl and R⁷ and R⁸ are the same or different and are selected from H and R⁶;
         R⁴ and R⁵ are the same or different and are selected from H and R³;
            or their racemic forms, their isomers and their pharmaceutically acceptable derivatives.

Of the compounds of formulae (I), (II) and (III) disclosed in WO 02/074754 particularly preferred are:
Spiro[cyclohexane-1-4′-(3′,4′-dihydro) quinazolin]-2′(1′H)-one, 6′-Methoxyspiro[cyclohexane-1-4′-(3′,4′-dihydro) quinazolin]-2′(1′H)-one, Spiro[cycloheptane-1-4′-(3′,4′-dihydro)quinazolin]-2′(1′H)-one, 7′-Methoxyspiro[cyclohexane-1-4′-(3′,4′-dihydro) quinazolin]-2′(1′H)-one, 6′-Phenylspiro[cycloheptane-1-4′-(3′,4′-dihydro) quinazolin]-2′(1′H)-one, 8′-Methoxyspiro[cyclohexane-1-4′-(3′,4′-dihydro) quinazolin]-2′(1′H)-one, 8′-Chlorospiro[cyclohexane-1-4′-(3′,4′-dihydro) quinazolin]-2′(1′H)-one, 7′-chlorospiro[cyclohexane-1-4′-(3′,4′-dihydro) quinazolin]-2′(1′H)-one, 5′-chlorospiro[cyclohexane-1-4′-(3′,4′-dihydro) quinazolin]-2′(1′H)-one, 8′-methylspiro[cyclohexane-1-4′-(3′,4′-dihydro) quinazolin]-2′(1′H)-one, 6′-chlorospiro[cyclohexane-1-4′-(3′,4′-dihydro) quinazolin]-2′(1′H)-one, 8′-bromospiro[cyclohexane-1-4′-(3′,4′-dihydro) quinazolin]-2′(1′H)-one, 8′-fluorospiro[cyclohexane-1-4′-(3′,4′-dihydro) quinazolin]-2′(1′H)-one, 6′-methylspiro[cyclohexane-1-4′-(3′,4′-dihydro) quinazolin]-2′(1′H)-one, 5′,8′-dichlorospiro[cyclohexane-1-4′-(3′,4′-dihydro) quinazolin]-2′(1′H)-one, 6′,7′-dichlorospiro[cyclohexane-1-4′-(3′,4′-dihydro) quinazolin]-2′(1′H)-one, 5′,6′-dichlorospiro[cyclohexane-1-4′-(3′,4′-dihydro) quinazolin]-2′(1′H)-one, 6′-phenylspiro[cyclohexane-1-4′-(3′,4′-dihydro) quinazolin]-2′(1′H)-one, 8′-iodospiro[cyclohexane-1-4′-(3′,4′-dihydro) quinazolin]-2′(1′H)-one, 8′-Bromospiro[cyclobutane-1-4′-(3,4′-dihydro)cquinazolin]-2′(1′H)-one, 8′-Bromospiro[cycloheptane-1-4′-(3,4′-dihydro)cquinazolin]-2′(1′H)-one, 8′-Bromo-4-methylspiro[cyclohexane-1-4′-(3′,4′-dihydro) qu(1′H)-one, 8′-Bromospiro[b]cyclo[3,2,1]octane-2-4′-(3′,4′-dihydro)quinazolin]-2′(1′H)-one, 6′,8′-dichlorospiro[cyclohexane-1-4′-(3′,4′-dihydro)quinazolin]-2′(1′H)-one 8′-chloro-6′-iodospiro[cyclohexane-1-4′-(3′,4′-dihydro)quinazolin]-2′(1′H)-one, 8′-chloro-6′-methoxyspiro[cyclohexane-1-4′-(3′,4′-dihydro) quinazolin]-2′(1′H)-one, 8′-chloro-6′-phenylspiro[cycloheptane-1-4′-(3′,4′-dihydro) quinazolin]-2′(1′H)-one, 8′-chloro-6′-phenylspiro[cyclohexane-1-4′-(3′,4′-dihydro) quinazolin]-2′(1′H)-one, 8′-chloro-6′-methylspiro[cyclohexane-1-4′-(3′,4′-dihydro) quinazolin]-2′(1′H)-one, 8′-chloro-6′-(3-pyridyl)spiro[cyclohexane-1-4′-(3′,4′-dihydro)quinazolin]-2′(1′H)-one, 8′-chloro-6-(4-pyridyl)spiro[cyclohexane-1-4′-(3′,4′-dihydro) quinazolin]-2′(1′H)one, 6′-(4-carboxyphenyl)-8′-chlorospiro[cyclohexane-1-4′-(3′,4′-dihydro)-quinazolin2′(1′H)-one, 6′-(3-carboxyphenyl)-8′-chlorospiro[cyclohexane-1-4′(3′,4′-dihydro)-quinazolin]2′(1′H)-one, 8′-chloro-6′-(1H-indol-5yl) spiro cyclohexane-1′4′-(3′,4′-dihydro)-quinazolin]2′(1′H)-one, 8′-chloro-6′-(2-pyridyl)spiro[cyclohexane-1-4′-(3′,4′-dihydro) quinazolin]-2′(1′H)one, 8′-chloro-6′-(3-dimethylamino-prop-1-ynyl)spiro[cyclohexane-1-4′-(3′,4′-dihydro)-quinazolin]-2′(1′H)-one, 8′-chloro-6′-(3-methylamino-prop-1-ynyl)spiro[cyclohexane-1-4′-(3′,4′-dihydro)quinazolin]-2′(1′H)-one, 8′-chloro-6′-[4-(4-methyl-piperazine-1-carbonyl)phenyl]spiro[cyclohexane-1-4′(3′,4′-dihydro) quinazolin]-2′(1′H)-one, 8′-chloro-6′-[4-(3-N-dimethylamino-propylcarboxamide) phenyl]-spiro-[cyclohexane-1-4′-(3′,4′-dihydro) quinazolin]-2′(1′H)-one, 8′-chloro-6′-[4-(2-N-dimethylamino-ethylcarboxamide)phenyl]-spiro-[cyclohexane1-4′-(3′,4′-dihydro)quinazolin[-2′(1′H)-one, 8′-chloro-6′-[3-(3-N-dimethylamino-propylcarboxamide) phenyl]-spiro-[cyclohexane-1-4′-(3′,4′-dihydro) quinazolin]-2′(1′H)-one, 8′-chloro-6′-[3-(4-methyl-piperazine-1-carbonyl)-phenyl]spiro-[cyclohexane-1-4′-(3′,4′-dihydro)quinazolin]-2′(1′H)-one, 8′-chloro-6′-[3-(2-N-dimethylamino-ethylcarboxamide) phenyl]spiro-[cyclohexane-1-4′-(3′,4′-dihydro) quinazolin]-2′(1′H)-one, 8′-Chlorospiro[cyclohexane-1-4′-(3′,4′-dihydro)quinazolin]-2′(1′H)-thione8′-Chloro-2′-cyanoiminospiro[cyclohexane-1-4′-(3′,4′-dihydro) quinazoline8′-Chloro-2′-methoxyiminospiro[cyclohexane-1-4′-(3′,4′-dihydro) quinazoline, 8′-Chloro-2′-dimethylaminospiro[cyclohexane-1-4′-(3′,4′-dihydro)quinazoline], 8′-Chloro-1′-methylspiro[cyclohexane-1-4′-(3′,4′-dihydro) quinazolin]-2′(1′H)-one, 8′-Chloro-1′-(ethoxycarbonylmethyl)spiro[cyclohexane-1-4′-(3′,4′-dihydro)-quinazolin]-2′(1′H)-one, 8′-Chloro-3′-methylspiro[cyclohexane-1-4′-(3′,4′-dihydro) quinazolin]-2′(1′H)-one, 8′-chloro-6′-[4-(4-pyrimidin-2-yl-piperazine-1-carbonyl)phenyl]spiro[-cyclohexanyl-4′-(3′,4′-dihydro) quinazolin]-2′(1′H)-one, 8′-chloro-6′-[4-(4-(2-morpholin-4-yl-ethyl)-piperazine-1-carbonyl)-phenyl]spiro[cyclohexane-1-4′-(3′,4′-dihydro) quinazolin]-2′(1′H)-one, 8′-chloro-6′-[4-(4-(2-morpholin-4-yl-2-oxo-ethyl)-piperazine-1-carbonyl)-phenyl]spiro[-cyclohexane-1-4′-(3′,4′-dihydro) quinazolin]-2′(1′H)-one, 8′-chloro-6′-[4-(4-(2-hydroxy-ethoxy)-ethyl)-piperazine-1-carbonyl)-phenyl]spiro[cyclohexane-1-4′-(3′,4′-dihydro) quinazolin]-2′(1′H)-one, 9′-Chlorospiro[cyclohexane-1-5′-(5′,10′-dihydro)]-imidazo[2,1-b]quinazoline9′-Chlorospiro[cyclohexane-1-5′-(5′,10′-dihydro)]-[1,2,4]triazolo[3,4-b]quinazoline, 9′-Chlorospiro[cyclohexane-1-5′-(4′,5′-dihydro)]-[1,2,4]triazolo[4,3-a]quinazoline, Spiro[cyclohexane-1-9′-(8′,9′-dihydro)-pyrazolo[4′,3′-f]quinazolin]-7′(6′H)-one, 8′-Chloro-5′-methoxyspiro[cyclohexane-1-4′-(3′,4′-dihydro) quinazolin]-2′(1′H)-one, 5′,8′-difluorospiro[cyclohexane-1-4′-(3′,4′-dihydro) quinazolin]-2′(1′H)-one, 8′-Chloro-5′-methylspiro[cyclohexane-1-4′-(3′,4′-dihydro) quinazolin]-2′(1′H)-one, 8′-Chloro-6′-(morpholin-4-yl)methylspiro[cyclohexane-1-4′-(3′,4′dihydro)quinazolin]-2′(1′H)-one, 8′-Chloro-5′-hydroxyspiro[cyclohexane-1-4′-(3′,4′-dihydro)quinazolin]-2′(1′H)-one, 8′-Chloro-5′-hydroxy-6′-iodo-spiro[cyclohexane-1-4′-(3′,-4′-dihydro)quinazolin]2′(1′H)-one, 8′-Chloro-6′-iodo-5′-methoxy-spiro[cyclohexane-1-4′-(3′,4′-dihydro) quinazolin]-2′(1′H)-one, 8′-chloro-6′-cyano-5′-methoxy-spiro[cyclohexane-1-4′-(3′,4′-dihydro)quinazolin]2′(1′H)-one, 8′-Chloro-5′-[2-(4-morpholino)ethoxy]spiro[cyclohexane-1-4′-(3′,4′dihydro) quinazolin]-2′(1′H)-one, 8′-Chloro-5′-[2-dimethylaminoethoxy]spiro[cyclohexane-1-4′-(3′,4′dihydro)quinazolin]-2′(1′H)-one, 8′-Chloro-5′(2-aminoethoxy)-spiro[cyclohexane-1-4′-(3′,4′-dihydro) quinazolin]2′(1′H)-one, 8′-Chloro-5′-[2-(methylamino)ethoxy]-spiro [cyclohexane-1′-4′(3′,4′dihydro)quinazolin]-2′(1′H)-one, 8-′Chloro-5′-[2-(2-aminoethoxy)ethoxy]spiro[cyclohexane-1-4′-(3′,4′dihydro)quinazolin]-2′(1′H)-one, 8′-Chloro-5′-[2-dimethylaminopropoxy]spiro[cyclohexane-1-4′-(3′,4′dihydro)quinazolin]-2′(1′H)-one, 8′-Chloro-5′-ethoxycarbonylmethoxyspiro[cyclohexane-1-4′-(3′,4′dihydro)quinazolin]-2′(1′H)-one, 5′-carboxymethoxy-8′-chloro-spiro[cyclohexane-1-4′-(3′,4′-dihydro) quinazolin]2′(1′H)-one, 5′-carboxypropoxy-8′-chloro-spiro[cyclohexane-1-4′-(3′,4′-dihydro) quinazolin]2′(1′H)-one, 8′-chloro-5′-(3-sulphopropoxy)-spiro[cyclohexane-1-4′-(3′,4′-dihydro) quinazolin2′(1′H)-one, 8′-Chloro-5′-[2-(tetrahydro-pyran-2-yloxy)-ethoxy]-spiro[cyclohexane-1-4′-(3′,4′dihydro)quinazolin]-2′(1′H)-one, 8′-Chloro-5′-(2-hydroxy-ethoxy)-spiro[cyclohexane-1-4′-(3′,4′-dihydro) quinazolin2′(1′H)-one, 8′-Chloro-5′-(5-ethoxycarbonyl-furan-2-ylmethoxy)-spiro[cyclohexane-1-4′-(3′,4′dihydro)quinazolin]-2′(1′H)-one, 8′-Chloro-5′-(5-carboxy-furan-2-ylmethoxy)-spiro[cyclohexane-1-4′-(3′,4′dihydro)quinazolin]-2′(1′H)-one, 8′-Chloro-5′-cyanomethoxyspiro[cyclohexane-1-4′-(3′,4′-dihydro) quinazolin]2′(1′H)-one, 8′-Chloro-5′-(1H-tetrazol-5-ylmethoxy)-spiro [cyclohexane-1-4′-(3′,4′dihydro)quinazolin]-2′(1′H)-one, 8′-Chloro-5′-(5-hydroxy-[1,2,4]oxadiazol-3-ylmethoxy)-spiro[cyclohexane-1-4′(3′,4′-dihydro) quinazolin]-2′(1′H)-one, 8′-Chloro-6′-iodo-5-′[2-dimethylamino-ethoxy]spiro[cyclohexane-1-4′-(3′,4′dihydro)quinazolin]-2′(1′H)-one, 6′-(4-carboxyphenyl)-8-′chloro-5′-methoxyspiro[cyclohexane-1-4′-(3′,4′dihydro) quinazolin]-2′(1′H)-one, 6′-(3-carboxyphenyl)-8′-chloro-5′-methoxyspiro[cyclohexane-1-4′-(3′,4′dihydro) quinazolin]-2′(1′H)-one, 8′-chloro-6′-[2-(4-methyl-piperazine-1-carbonyl)phenyl]spiro[cyclohexane-1-4′(3′,4′-dihydro) quinazolin]-2′(1′H)-one, <RTI 8′-chloro-6′-[2-methyl-4-(4-methyl-piperazine-1-carbonyl)phenyl]spiro[cyclohexane-1-4′-(3′,4′-dihydro) quinazolin]-2′(1′H)-one, 8′-chloro-6′-[4-(piperazine-1-carbonyl)phenyl]spiro[cyclohexane-1-4′-(3′,4′dihydro)quinazolin]-2′(1′H)-one, 8′-chloro-6′-[4-carbamoyl-phenyl]spiro[cyclohexane-1-4′-(3′,4′-dihydro) quinazolin]2′(1′H)-one, 8′-chloro-6′-[4-(1-methyl-piperidin-4-yl)-piperazine-1-carbonyl)phenyl]spiro[cyclohexane-1-4′-(3′,4′-dihydro) quinazolin]-2′(1′H)-one, 8′-chloro-5′-methoxy-6′-[4-(4-methyl-piperazine-1-carbonyl)phenyl]spiro[cyclohexane-1-4′-(3′,4′-dihydro) quinazolin]-2′(1′H)-one, 8-Chloro-5-methoxyspiro[4H-benzo[d][1,3]oxazin-2-ylamino-4-4′-(tetrahydropyran-4′-yl)], 8′-Trifluoromethylspiro[cyclohexane-1-4′-(3′,4′-dihydro) quinazolin]-2′(1′H)-one, 8′-Chloro-6′-cyanomethylspiro[cyclohexane-1-4′-(3′,4′-dihydro)quinazolin]-2′(1′H)-one, 8′-chloro-5′-(3-dimethylamino-2-hydroxy-propoxy)-spiro[cyclohexane-1-4′-(3′,4′dihydro)quinazolin]-2′(1′H)-one, 8′-Chloro-5′-(3-methylamino-2-hydroxy-propoxy)-spiro [cyclohexane-1-4′-(3′,4′dihydro) quinazolin]-2′(1′H)-one, 8′-Chloro-5′-[2-(ethoxycarbonylmethyl-amino)-ethoxy]-spiro[cyclohexane-1-4′-(3′,4′-dihydro) quinazolin]-2′(1′H)-one, 8′-Chloro-5′-[2-(carboxymethyl-amino)-ethoxy]-spiro [cyclohexane-1-4′-(3′, 4 dihydro) quinazolin]-2′(1′H)-one hydrochloride, 8′-Chloro-5′-(2-methanesulfonylamino-2-oxo-ethoxy)-spiro[cyclohexane-1-4′-(3′,4′-dihydro)quinazolin]-2′(1′H)-one, 8′-Chloro-5′-(2-[(5-methyl-isoxazol-3-ylmethyl)-amino]ethoxy)-spiro [cyclohexan1-4′-(3′,4′-dihydro) quinazolin]-2′(1′H)-one, 8′-bromospiro[cyclohexane-1-4′-(3′,4′-dihydro) quinazolin]-2′(1′H)-one, 5′,8′-dichlorospiro[cyclohexane-1-4′-(3′,4′-dihydro) quinazolin]-2′(1′H)-one, 8′-Bromospiro[cycloheptane-1-4′-(3′,4′-dihydro) quinazolin]-2′(1′H)-one, 8′-chloro-6′-methoxyspiro[cyclohexane-1-4′-(3′,4′-dihydro) quinazolin]-2′(1′H)-one, 8′-chloro-6′-phenylspiro[cyclohexane-1-4′-(3′,4′-dihydro) quinazolin]-2′(1′H)-one, 8′-chloro-6′-(3-pyridyl)spiro[cyclohexane-1-4′-(3′,4′-dihydro)quinazolin]-2′(1′H)— one, 8′-chloro-6′-(4-pyridyl)spiro[cyclohexane-1-4′-(3′,4′-dihydro) quinazolin]-2′(1′H)one, 6′-(4-carboxyphenyl)-8′-chlorospiro[cyclohexane-1-4′-(3′,4′-dihydro)-quinazolin]2′(1′H)-one, 6′-(3-carboxyphenyl)-8′-chlorospiro[cyclohexane-1-4′-(3′,4′-dihydro)-quinazolin]2′(1′H)-one, 8′-chloro-6′-(1H-indol-5-yl)spiro[cyclohexane-1-4′-(3′,4′-dihydro)-quinazolin]-2′(1′H)-one, 8′-chloro-6′-(2-pyridyl)spiro[cyclohexane-1-4′-(3′,4′-dihydro)quinazolin]-2′(1′H)-one, 8′-chloro-6′-(3-dimethylamino-prop-1-ynyl)spiro[cyclohexane-1-4′-(3′,4′-dihydro)-quinazolin]-2′(1′H)-one, 8′-chloro-6′-(3-methylamino-prop-1-ynyl)spiro[cyclohexane-(3′,4′dihydro)quinazolin]-2′(1′H)-one, 8′-chloro-6′-[4-{4-methyl-piperazine-1-carbonyl)phenyl]spiro[cyclohexane-1-4′(3′,4′-dihydro)quinazolin]-2′(1′H)-one, 8′-chloro-6′-[4-(3-N-dimethylamino-propylcarboxamide)phenyl]-spiro[cyclohexane-1-4′-(3′,4′-dihydro) quinazolin]-2′(1′H)-one, 8′-chloro-6′-[4-(2-N-dimethylamino-ethylcarboxamide) phenyl]-spiro-[cyclohexane-1-4′-(3′,4′-dihydro) quinazolin]-2′(1′H)-one, 8′-chloro-6′-[3-(3-N-dimethylamino-propylcarboxamide) phenyl]-spiro-[cyclohexane-1-4′-(3′,4′-dihydro) quinazolin]-2′(1′H)-one, 8′-chloro-6′-[3-(4-methyl-piperazine-1-carbonyl)-phenyl]spiro-[cyclohexane-1-4′(3′,4′-dihydro)quinazolin]-2′(1′H)-one, 8′-chloro-6′-[3-(2-N-dimethylamino-ethylcarboxamide) phenyl]spiro-[cyclohexane-1-4′-(3′,4′-dihydro) quinazolin]-2′(1′H)-one, 8′-chloro-6′-[4-(4-pyrimidin-2-yl-piperazine-1-carbonyl)phenyl]spiro[-cyclohexanyl-4′-(3′,4′-dihydro) quinazolin]-2′(1′H)-one, 8′-chloro-6′-[4-(4-(2-morpholin-4-yl-ethyl)-piperazine-1-carbonyl)-phenyl]spiro[cyclohexane-1-4′-(3′,4′-dihydro) quinazolin]-2′(1′H)-one, 8′-chloro-6′-[4-(4-(2-morpholin-4-yl-2-oxo-ethyl)-piperazine-1-carbonyl)-phenyl]spiro [cyclohexane-1-4′-(3′,4′-dihydro) quinazolin]-2′(1′H)-one, 8′-chloro-6′-[4-(4-(2-hydroxy-ethoxy)-ethyl)-piperazine-1-carbonyl)-phenyl]spiro[cyclohexane-1-4′(3-,4′-dihydro) quinazolin]-2′(1′H)-one, 8′-Chloro-5′-methoxyspiro[cyclohexane-1-4-(3′,4′-dihydro) quinazolin]-2′(1′H)-one, 8′-Chloro-5′-methylspiro[cyclohexane-1-4′-(3′,4′-dihydro) quinazolin]-2′(1′H)-one, 8′-Chloro-5′-hydroxyspiro[cyclohexane-1-4′-(3′,4′-dihydro) quinazolin]-2′(1′H)-one, 8′-Chloro-6′-cyano-5′-methoxy-spiro[cyclohexane-1-4′-(3′,4′-dihydro) quinazolin]2′(1′H)-one, 8′-Chloro-5′-[2-(4-morpholino) ethoxy]spiro(cyclohexane-1-4′-(3′,4′dihydro)quinazolin]-2′(1′H)-one, 5′-carboxymethoxy-8′-chloro-spiro[cyclohexane-1-4′-(3′,4′-dihydro) quinazolin2′(1′H)-one, 5′-carboxpropoxy-8′-chloro-spiro[cyclohexane-1-4′-(3′,4′-dihydro) quinazolin2′(1′H)-one, 8′-chloro-5′-(3-sulphopropoxy)-spiro[cyclohexane-1-4′-(3′,4′-dihydro) quinazolin]2′(1′H)-one, 8′-Chloro-5′-(2-hydroxy-ethoxy)-spiro[cyclohexane-1-4′-(3′,4′-dihydro)quinazolin]2′(1′H)-one, 8′-Chloro-5-(5-ethoxycarbonyl-furan-2-ylmethoxy)-spiro[cyclohexane-1-4′-(3′,4′dihydro)quinazolin]-2′(1′H)-one, 8′-Chloro-5′-(5-carboxy-furan-2-ylmethoxy)-spiro [cyclohexane-1-4′-(3′,4dihydro) quinazolin]-2′(1′H)-one, 8′-Chloro-5′-cyanomethoxyspiro[cyclohexane-1-4′-(3′,4′-dihydro)quinazolin]2′(1′H)-one, 8′-Chloro-5′-(1H-tetrazol-5-ylmethoxy)-spiro(cyclohexane-1-4′-(3′,4′-dihydro)quinazolin]-2′(1′H)-one, 8′-Chloro-5′-(5-hydroxy-[1,2,4]oxadiazol-3-ylmethoxy)-spiro[cyclohexane-1-4′(3′,4′-dihydro) quinazolin]-2′(1′H)-one, 6′-(4-carboxyphenyl)-8′-chloro-5′-methoxyspiro[cyclohexane-1-4′-(3′,4dihydro) quinazolin]-2′(1′H)-one, 6′-(3-carboxyphenyl)-8′-chloro-5′-methoxyspiro[cyclohexane-1-4′-(3′,4′dihydro)quinazolin]-2′(1′H)-one, 8′-chloro-6′-[2-methyl-4-(4-methyl-piperazine-1 carbonyl)phenyl]spiro[cyclohexane-1-4′-(3′,4′-dihydro)quinazolin]-2′(1′H)-one, 8′-chloro-6′-[4-(piperazine-1-carbonyl)phenyl]spiro[cyclohexane-1-4′-(3′,4′dihydro) quinazolin]-2′(1′H)-one, 8′-chloro-6′-[4-carbamoyl-phenyl]spiro[cyclohexane-1-4′-(3′,4′-dihydro)quinazolin]2′(1′H)-one, 8′-chloro-6′-[4-((1-methyl-piperidin-4-yl)-piperazine-1-carbonyl)phenyl]spiro[cyclohexane-1-4′-(3′,4′-dihydro) quinazolin]-2′(1′H)-one, and, 8′-Chloro-5′-[2-(carboxymethyl-amino)-ethoxy]-spiro[cyclohexane-1-4′-(3′,4′dihydro)quinazolin]-2′(1′H)-one hydrochloride, 8′-Chloro-5′-(2-methanesulfonylamino-2-oxo-ethoxy)-spiro[cyclohexane-1-4′-(3′,4′-dihydro)quinazolin]-2′(1′H)-one, 8′-Chloro-5′-(2-[(5-methyl-isoxazol-3-ylmethyl)-amino]ethoxy)-spiro[cyclohexanel-4′-(3′,4′-dihydro)quinazolin]-2′(1′H)-one, optionally in combination with an appropriate carrier.

The following compounds of Formula (IV) are most preferred:
cis-3-[(8′-Chloro-2′-oxo-2′,3′-dihydro-1′H-spiro[cyclohexane-1,4′-quinazolin]-5′-yl)oxy]cyclobutanecarboxylic acid; trans-3-[(8′-Chloro-2′-oxo-2′,3′-dihydro-1′H-spiro[cyclohexane-1,4′-quinazolin]-5′-yl)oxy]cyclobutanecarboxylic acid; and pharmaceutically acceptable salts, solvates and prodrugs thereof.

Of the compounds of formulae (V) disclosed in WO 04/026818, particularly preferred are 5′-(2-[(2-amino-2-oxoethyl)amino]ethoxy)-8′-chloro-1′H-spiro[cyclohexane-1,4′-quinazolin]-2′(3′H)-one; 8′-chloro-5′-([methylsulfinyl]methoxy)-1′H-spiro[cyclohexane-1,4′-quinazolin]-2′(3′H)-one; 5′-(2-{[2-(acetylamino)ethyl]amino}ethoxy)-8′-chloro-1′H-spiro[cyclohexane-1,4′-quinazolin]-2′(3′H)-one; 8′-fluoro-5′-[3-(methylsulfinyl)propoxy]-1′H-spiro[cyclohexane-1,4′-quinazolin]-2′(3′H)-one; 8′-fluoro-5′-([methylsulfinyl]methoxy)-1′H-spiro[cyclohexane-1,4′-quinazolin]-2′(3′H)-one, and, 8′-fluoro-5′-(2-{[1-(1H-pyrazol-3-ylmethyl)azetidin-3-yl]oxy}1′H-spiro[cyclohexane-1,4′-quinazolin]-2′(3′H)-one.

Further examples of suitable PDE7 inhibitors for use in the invention include those compounds generally or specifically disclosed in the PCT published patent application WO02/28847 (Warner Lambert) which discloses compounds of Formula (VI)

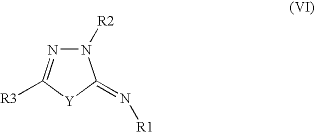

in which
Y is O or S;
R1 is:
   C₄-C₁₀ alkyl,
   C₂-C₁₀ alkenyl,
   C₂-C₁₀ alkynyl,
   cycloalkyl,
   cycloalkenyl,
   heterocycle,
   aryl,
   or a bicyclic group;
      each optionally substituted with one or several groups X₁—R₄, identical or different, in which:
X₁ is:
   a single bond, lower alkylene, C₂-C₆ alkenylene, cycloalkylene, arylene or divalent heterocycle, and,
R₄ is:
   1) H, ═O, NO₂, CN, halogen, lower haloalkyl, lower alkyl, carboxylic acid bioisostere,
   2) COOR₅, C(═O)R₅, C(═S)R₅, SO₂R₅, SOR₅, SO₃R₅, SR₅, OR₅,
   3) C(═O)NR₇R₈, C(═S)NR₇R₃, C(═CH—NO₂)NR₇R₃, C(═N—CN)NR₇R₈, C(═N—SO₂NH₂)NR₇R₅, C(═NR₇)NHR₃, C(═NR₇)R₃, C(═NR₉)NHR₃, C(═NR₉)R₈, SO₂NR₇R₈ or NR₇R₃ in which R₇ and R₈ are the same or different and are selected from OH, R₅, R₆, C(═O)NR₅R₆, C(═O)R₅, SO₂R₅, C(═NR₉)NHR₁₀, C(═NR₉)R₁₀, C(═CH—NO₂)NR₉R₁₀, C(═N—SO₂NH₂)NR₉R₁₀, C(═N—CN)NR₉R₁₀ or C(═S)NR₉R₁₀;
R₂ is:
   lower alkyl,
   C₂-C₁₀ alkenyl,
   C₄-C₁₀ alkynyl,
   cycloalkyl,
   cycloalkenyl,
   heterocycle,
   aryl;
      each optionally substituted with one or several groups which are the same or different and which are selected from:
1) H, carboxylic acid bioisostere, lower haloalkyl, halogen,
2) COOR₅, OR₅, SO₂R₅,
3) SO₂NR₁₁R₁₂, C(═O)NR₁₁R₁₂ or NR₁₁R₁₂ in which R₁₁ and R₁₂ are the same or different and are selected from OH, R₅, R₆, C(═O)NR₅R₃, C(═O)R₅, SO₂R₅, C(═S)NR₉R₁₀, C(═CH—NO₂)NR₉R₁₀, C(═N—CN)NR₉R₁₀, C(═N—SO₂NH₂)NR₉R₁₀, C(═NR₉)NHR₁₀ or C(═NR₉)R₁₀;
R₃ is X₂—R′₃ wherein:
   X₂ is a single bond or, a group selected from C₁-C₄ alkylene, C₂-C₆ alkenylene, C₂-C₆ alkynylene, each optionally substituted with one or several groups which are the same or different and which are selected from:
      1) H, C₁-C₃ alkyl, C₃-C₄ cycloalkyl, aryl, heterocycle, ═O, CN,
      2) OR₅, ═NR₅ or,
      3) NR₁₃R₁₄ in which R₁₃ and R₁₄ are the same or different and are selected from R₅, R₆, C(═O)NR₅R₆, C(═O)R₅, SO₂R₅, C(═S)NR₉R₁₀, C(═CH—NO₂)NR₉R₁₀, C(═NR₉)NHR₁₀ or C(═NR₉)R₁₀;
   R₁₃ is:
   cycloalkyl,
   cycloalkenyl,
   aryl,
   heterocycle,
   or a polycyclic group;
      each optionally substituted with one or several groups X₃—R₁₇, identical or different, in which:
X₃ is:
   a single bond, lower alkylene, C₂-C₆ alkenylene, C₂-C₆ alkynylene, cycloalkylene, arylene, divalent heterocycle or a divalent polycyclic group, and,
R₁₇ is:
   1) H, ═O, NO₂, CN, lower haloalkyl, halogen, cycloalkyl,
   2) COOR₅, C(═O)R₅, C(═S)R₅, SO₂R₅, SOR₅, SO₃R₅, SR₅, OR₅,
   3) C(═O)NR₁₅R₁₆, C(═S)NR₁₅R₁₆, C(═N—CN)NR₁₅R₁₆, C(═N—SO₂NH₂)NR₁₅R₁₆, C(═CH—NO₂)NR₁₅R₁₆, SO₂NR₁₅R₁₆, C(═NR₁₅)NHR₁₆, C(═NR₁₅)R₁₆, C(═NR₉)NHR₁₆, C(═NR₉)R₁₆ or NR₁₅R₁₆ in which R₁₅ and R₁₆ are the same or different and are selected from OH, R₅, R₆, C(═O)NR₅R₃, C(═O)R₅, SO₂R₅, C(═S)NR₉R₁₀, C(═CH—NO₂)NR₉R₁₀, C(═N—CN)NR₉R₁₀, C(═N—SO₂NH₂)NR₉R₁₀, C(═NR₉)NHR₁₀ or C(═NR₉)R₁₀
   4) heterocycle optionally substituted with one or several groups R₅;
R₅ and R₆ are the same or different and are selected from:
   H,
   lower alkyl, C₂-C₆ alkenyl, C₂-C₆ alkynyl;
   X₄-cycloalkyl, X₄-cycloalkenyl, X₄-aryl, X₄-heterocycle or X₄-polycyclic group, in
      which X₄ is a single bond, lower alkylene or C₂-C₆ alkenylene;
each optionally substituted with one or several groups which are the same or different and which are selected from:
   halogen, ═O, COOR₂₀, CN, OR₂₀, lower alkyl optionally substituted with OR₂₀, O-lower alkyl optionally substituted with OR₂₀, C(═O)-lower alkyl, lower haloalkyl,

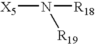

which X₅ is a single bond or lower alkylene and R₁₈, R₁₉ and R₂₀ are the same or different and are selected from H or lower alkyl;
X₆-heterocycle, X₆-aryl, X₆-cycloalkyl, X₆-cycloalkenyl, X₆-polycyclic group in which X₆ is selected from a single bond or lower alkylene, these groups being optionally substituted with one or several groups, identical or different, selected from halogens, COOR₂₁, OR₂₁, or (CH₂)ₙNR₂₁R₂₂ in which n is 0, 1 or 2 and R₂₁ and R₂₂ are the same or different and are selected from H or lower alkyl;

R₉ is selected from H, CN, OH, lower alkyl, O-lower alkyl, aryl, heterocycle, SO₂NH₂

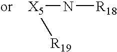

in which X₅ is a single bond or lower alkylene and R₁₈ and R₁₉ are the same or different and are selected from H or lower alkyl;
R₁₀ is selected from hydrogen, lower alkyl, cyclopropyl or heterocycle;
   or a pharmaceutically acceptable derivative thereof,
with the proviso that,
   when R1 is phenyl, it bears at least one substituent other than H,
   when X₂ is a single bond and both R₁ and R′₃ are phenyl, each of R1 and R′₃ bear at least one substituent other than H,
   when X₂ is a single bond and R₁₃ is phenyl, R₁₃ is not substituted by an ester or a carboxylic acid in the ortho position,
   the atom of R3 which is linked to the thiadiazole group is a carbon atom, with the exclusion of the following compounds,
1-Phenyl-1-[4-phenyl-5-(5-trifluoromethyl-2H-[1,2,4]triazol-3-ylimino)-4,5-dihydro-[1,3,4]thiadiazol-2-yl]-methanone, 1-[4-Phenyl-5-(5-trifluoromethyl-2H-[1,2,4]triazol-3-ylimino)-4,5-dihydro-[1,3,4]thiadiazol-2-yl]-1-thiophen-2-yl-methanone, 1-Phenyl-1-(4-phenyl-5-p-tolylimino-4,5-dihydro-[1,3,4]thiadiazol-2-yl)-methanone, Cyclohexyl-[3-(2,4,6-trichloro-phenyl)-5-(2,3,3-trimethyl-cyclopent-1-enylmethyl)-3H-[1,3,4]thiadiazol-2-ylidene]-amine, 2-(3,5-Diphenyl-3H-[1,3,4]thiadiazol-2-ylideneamino)-1,4-diphenyl-but-2-ene-1,4-dione, 2-[3-Phenyl-5-(1-phenyl-methanoyl)-3H-[1,3,4]thiadiazol-2-ylideneamino]-but-2-enedioic acid dimethyl ester, 2-[5-(1-Phenyl-methanoyl)-3-p-tolyl-3H-[1,3,4]thiadiazol-2-ylideneamino]-but-2-enedioic acid dimethyl ester, and, 2-[3-(4-Chloro-phenyl)-5-(1-phenyl-methanoyl)-3H-[1,3,4]thiadiazol-2-ylideneamino]-but-2-enedioic acid dimethyl ester.

Of the compounds of formula (VI) disclosed in WO02/28847, particularly preferred are: compounds selected from the group consisting of: 3-[5-(4-Chloro-phenyl)-3-methyl-3H-[1,3,4]thiadiazol-2-ylideneamino]-benzoic acid, (1R*,2R*)-2-[5-(4-Chloro-phenyl)-3-methyl-3H-[1,3,4]thiadiazol-2-ylideneamino]-cyclohexanecarboxylic acid, (S)-2-[5-(4-Chloro-phenyl)-3-methyl-3H-[1,3,4]thiadiazol-2-ylideneamino]-2-phenyl-ethanol, 2-{2-[5-(4-Chloro-phenyl)-3-methyl-3H-[1,3,4]thiadiazol-2-ylideneamino]-phenyl}-ethanol, {1-[5-(4-Chloro-phenyl)-3-methyl-3H-[1,3,4]thiadiazol-2-ylideneamino]-cyclopentyl}-methanol, 3-[5-(4-Chloro-phenyl)-3-methyl-3H-[1,3,4]thiadiazol-2-ylideneamino]-cyclohexanecarboxylic acid, 5-[5-(4-Chloro-phenyl)-3-methyl-3H[1,3,4]thiadiazol-2-ylideneamino]-2-fluoro-benzoic acid, 3-[5-(4-Chloro-phenyl)-3-methyl-3H-[1,3,4]thiadiazol-2-ylideneamino]-2,5,6-trifluoro-benzoic acid, [5-(4-Chloro-phenyl)-3-methyl-3H-[1,3,4]thiadiazol-2-ylidene]-propyl-amine, (S)-2-[5-(4-Chloro-phenyl)-3-methyl-3H-[1,3,4]thiadiazol-2-ylideneamino]-butan-1-ol, [5-(4-Chloro-phenyl)-3-methyl-3H-[1,3,4]thiadiazol-2-ylidene]-cyclobutyl-amine, 3-[5-(4-Chloro-phenyl)-3-methyl-3H-[1,3,4]thiadiazol-2-ylideneamino]-azepan-2-one, [5-(4-Chloro-phenyl)-3-methyl-3H-[1,3,4]thiadiazol-2-ylidene]-cyclopentyl-amine, [5-(4-Chloro-phenyl)-3-methyl-3H-[1,3,4]thiadiazol-2-ylidene]-cycloheptyl-amine, (S)-2-[5-(4-Chloro-phenyl)-3-methyl-3H-[1,3,4]thiadiazol-2-ylideneamino]-3-methyl-butan-1-ol, 2-[5-(4-Chloro-phenyl)-3-methyl-3H-[1,3,4]thiadiazol-2-ylideneamino]-2-methyl-propan-1-ol, tert-Butyl-[5-(4-chloro-phenyl)-3-methyl-3H-[1,3,4]thiadiazol-2-ylidene]-amine, [5-(4-Chloro-phenyl)-3-methyl-3H-[1,3,4]thiadiazol-2-ylidene]-isopropyl-amine, 4-[5-(4-Chloro-phenyl)-3-methyl-3H-[1,3,4]thiadiazol-2-ylideneamino]-benzoic acid, [5-(4-Chloro-phenyl)-3-methyl-3H-[1,3,4]thiadiazol-2-ylidene]-(1-ethyl-propyl)-amine, 4-[5-(4-Chloro-phenyl)-3-methyl-3H-[1,3,4]thiadiazol-2-ylideneamino]-phenol, N-[5-(4-Chloro-phenyl)-3-methyl-3H-[1,3,4]thiadiazol-2-ylidene]-cyclohexane-1,2-diamine, [5-(4-Chloro-phenyl)-3-methyl-3H-[1,3,4]thiadiazol-2-ylidene]-(4-fluoro-phenyl)-amine, N-[5-(4-Chloro-phenyl)-3-methyl-3H-[1,3,4]thiadiazol-2-ylidene]-cyclohexane-1,4-diamine, (1R*,2S*)-2-[5-(4-Chloro-phenyl)-3-methyl-3H-[1,3,4]thiadiazol-2-ylideneamino]-cyclohexanol, [5-(4-Chloro-phenyl)-3-methyl-3H-[1,3,4]thiadiazol-2-ylidene]-(4-trifluoromethyl-phenyl)-amine, 3-[5-(4-Methanesulfonyl-phenyl)-3-methyl-3H-[1,3,4]thiadiazol-2-ylideneamino]-benzoic acid, 3-[5-(4-Chloro-phenyl)-3-methyl-3H-[1,3,4]thiadiazol-2-ylideneamino]-phenol, 5-[5-(4-Chloro-phenyl)-3-methyl-3H-[1,3,4]thiadiazol-2-ylideneamino]-2-hydroxy-benzoic acid, (1-Aza-bicyclo[2.2.2]oct-3-yl)-[5-(4-chloro-phenyl)-3-methyl-3H-[1,3,4]thiadiazol-2-ylidene]-amine, 2-[5-(4-Chloro-phenyl)-3-methyl-3H-[1,3,4]thiadiazol-2-ylideneamino]-phenol, (R)-2-[5-(4-Chloro-phenyl)-3-methyl-3H-[1,3,4]thiadiazol-2-ylideneamino]-butan-1-ol, [5-(4-Chloro-phenyl)-3-methyl-3H-[1,3,4]thiadiazol-2-ylidene]-(3-fluoro-phenyl)-amine, (3-Chloro-phenyl)-[5-(4-chloro-phenyl)-3-methyl-3H-[1,3,4]thiadiazol-2-ylidene]-amine, {3-[5-(4-Chloro-phenyl)-3-methyl-3H-[1,3,4]thiadiazol-2-ylideneamino]-phenyl}-acetic acid, 3-[5-(4-Chloro-phenyl)-3-methyl-3H-[1,3,4]thiadiazol-2-ylideneamino]-benzamide, Bicyclo[2.2.1]hept-2-yl-[5-(4-chloro-phenyl)-3-methyl-3H-[1,3,4]thiadiazol-2-ylidene]-amine, (1R*,2R*)-2-[5-(4-Chloro-phenyl)-3-methyl-3H-[1,3,4]thiadiazol-2-ylideneamino]-cyclohexanol, 5-(5-Cyclohexyl-3-methyl-3H-[1,3,4]thiadiazol-2-ylideneamino)-2-methoxy-phenol, 3-(5-Cyclohexyl-3-methyl-3H-[1,3,4]thiadiazol-2-ylideneamino)-benzoic acid, 3-[5-(4-Chloro-phenyl)-3-methyl-3H-[1,3,4]thiadiazol-2-ylideneamino]-4-hydroxy-benzoic acid, (5-Cyclohexyl-3-methyl-3H-[1,3,4]thiadiazol-2-ylidene)-(3-methanesulfonyl-phenyl)-amine, (1R*,2R*)-2-[5-(4-Methanesulfonyl-phenyl)-3-methyl-3H-[1,3,4]thiadiazol-2-ylideneamino]-cyclohexanol, Cyclohexyl-[5-(2,4-dichloro-phenyl)-3-methyl-3H-[1,3,4]thiadiazol-2-ylidene]-amine, [5-(2-Chloro-phenyl)-3-methyl-3H-[1,3,4]thiadiazol-2-ylidene]-cyclohexyl-amine, Cyclohexyl-[3-methyl-5-(4-trifluoromethyl-phenyl)-3H-[1,3,4]thiadiazol-2-ylidene]-amine, Cyclohexyl-(3-methyl-5-pyridin-4-yl-3H-[1,3,4]thiadiazol-2-ylidene)-amine, [5-(3-Chloro-phenyl)-3-methyl-3H-[1,3,4]thiadiazol-2-ylidene]-cyclohexyl-amine, 4-(5-Cyclohexylimino-4-methyl-4,5-dihydro-[1,3,4]thiadiazol-2-yl)-benzonitrile, Cyclohexyl-[5-(4-methanesulfonyl-phenyl)-3-methyl-3H-[1,3,4]thiadiazol-2-ylidene]-amine, [3-(5-Cyclohexylimino-4-methyl-4,5-dihydro-[1,3,4]thiadiazol-2-yl)-phenyl]-dimethyl-amine, Cyclohexyl-[5-(3-methoxy-4-nitro-phenyl)-3-methyl-3H-[1,3,4]thiadiazol-2-ylidene]-amine, 2,4-Dichloro-5-(5-cyclohexylimino-4-methyl-4,5-dihydro-[1,3,4]thiadiazol-2-yl)-benzenesulfonamide, Cyclohexyl-(3-methyl-5-thiophen-3-yl-3H-[1,3,4]thiadiazol-2-ylidene)-amine, Cyclohexyl-[5-(3,5-dichloro-phenyl)-3-methyl-3H-[1,3,4]thiadiazol-2-ylidene]-amine, Cyclohexyl-[5-(2-ethyl-5-methyl-2H-pyrazol-3-yl)-3-methyl-3H-[1,3,4]thiadiazol-2-ylidene]-amine, [5-(3-Chloro-2,6-dimethoxy-phenyl)-3-methyl-3H-[1,3,4]thiadiazol-2-ylidene]-cyclohexyl-amine, Cyclohexyl-(5-isoxazol-5-yl-3-methyl-3H-[1,3,4]thiadiazol-2-ylidene)-amine, Cyclohexyl-[3-methyl-5-(5-pyridin-2-yl-thiophen-2-yl)-3H-[1,3,4]thiadiazol-2-ylidene]-amine, 5-(5-Cyclohexylimino-4-methyl-4,5-dihydro[1,3,4]thiadiazol-2-yl)-2-methoxy-benzene-1,3-diol; compound with trifluoro-methanesulfonic acid, 5-(5-Cyclohexylimino-4-methyl-4,5-dihydro[1,3,4]thiadiazol-2-yl)-2,3-dimethoxy-phenol, compound with trifluoro-methanesulfonic acid [5-(4-Chloro-phenyl)-3-methyl-3H-[1,3,4]thiadiazol-2-ylidene]-cyclohexyl-amine, 2-Chloro-4-(5-cyclohexylimino-4-methyl-4,5-dihydro-[1,3,4]thiadiazol-2-yl)-6-methoxy-phenol; compound with 1,1,1-trifluoro-methanesulfonic acid, 2-Chloro-5-(5-cyclohexylimino-4-methyl-4,5-dihydro-[1,3,4]thiadiazol-2-yl)-benzenesulfonamide, 2-Chloro-5-(5-cyclohexylimino-4-methyl-4,5-dihydro[1,3,4]thiadiazol-2-yl)-N,N-diethyl-benzenesulfonamide, {5-[4-Chloro-3-(4-methyl-piperazine-1-sulfonyl)-phenyl]-3-methyl-3H-[1,3,4]thiadiazol-2-ylidene}-cyclohexyl-amine, 2-Chloro-5-(5-cyclohexylimino-4-methyl-4,5-dihydro-[1,3,4]thiadiazol-2-yl)-N-pyridin-4-ylmethyl-benzenesulfonamide, 2-Chloro-5-(5-cyclohexylimino-4-methyl-4,5-dihydro-[1,3,4]thiadiazol-2-yl)-N-(2-morpholin-4-yl-ethyl)-benzenesulfonamide, 2-Chloro-5-(5-cyclohexylimino-4-methyl-4,5-dihydro-[1,3,4]thiadiazol-2-yl)-N-ethyl-benzenesulfonamide, 2-Chloro-5-(5-cyclohexylimino-4-methyl-4,5-dihydro-[1,3,4]thiadiazol-2-yl)-N-ethyl-N-(2-morpholin-4-yl-ethyl)-benzenesulfonamide, 2-Chloro-5-(5-cyclohexylimino-4-methyl-4,5-dihydro-[1,3,4]thiadiazol-2-yl)-N-isopropyl-N-(2-morpholin-4-yl-ethyl)-benzenesulfonamide, 2-Chloro-5-(5-cyclohexylimino-4-methyl-4,5-dihydro-[1,3,4]thiadiazol-2-yl)-N-ethyl-N-[2-(2-methoxy-ethoxy)-ethyl]-benzenesulfonamide, 2-Chloro-5-(cyclohexylimino-methyl-4,5-dihydro-[1,3,4]thiadiazol-2-yl)-N-(3-dimethylamino-2-hydroxy-propyl)-N-ethyl-benzenesulfonamide, 2-Chloro-5-(5-cyclohexylimino-4-methyl-4,5-dihydro-[1,3,4]thiadiazol-2-yl)-N-(2,3-dihydroxy-propyl)-N-ethyl-benzenesulfonamide, 2-Chloro-5-(5-cyclohexylimino-4-methyl-4,5-dihydro-[1,3,4]thiadiazol-2-yl)-N-ethyl-N-(2-hydroxy-3-pyrrolidin-1-yl-propyl)-benzenesulfonamide, 2-Chloro-5-(cyclohexylimino-methyl-4,5-dihydro-[1,3,4]thiadiazol-2-yl)-N-(2-diethylamino-ethyl)-N-ethyl-benzenesulfonamide, 2-Chloro-5-(5-cyclohexylimino-4-methyl-4,5-dihydro-[1,3,4]thiadiazol-2-yl)-N-(2-dimethylamino-propyl)-N-ethyl-benzenesulfonamide, [5-(4-Chloro-phenyl)-2-cyclohexylimino-[1,3,4]thiadiazol-3-yl]-acetic acid methyl ester, 3-(5-Cyclohexylimino-4-methyl-4,5-dihydro-[1,3,4]thiadiazol-2-yl)-benzoic acid methyl ester, 3-(5-Cyclohexylimino-4-methyl-4,5-dihydro-[1,3,4]thiadiazol-2-yl)-benzoic acid, 3-(5-Cyclohexylimino-4-methyl-4,5-dihydro-[1,3,4]thiadiazol-2-yl)-benzamide, 3-(5-Cyclohexylimino-4-methyl-4,5-dihydro-[1,3,4]thiadiazol-2-yl)-N-(2-hydroxy-ethyl)-benzamide, 3-(5-Cyclohexylimino-4-methyl-4,5-dihydro-[1,3,4]thiadiazol-2-yl)-N-methyl-benzamide, 4-(5-Cyclohexylimino-4-methyl-4,5-dihydro-[1,3,4]thiadiazol-2-yl)-benzene-1,2-diol, 4-(5-Cyclohexylimino-4-methyl-4,5-dihydro-[1,3,4]thiadiazol-2-yl)-2,6-dimethoxy-phenol, 6-(5-Cyclohexylimino-4-methyl-4,5-dihydro-[1,3,4]thiadiazol-2-yl)-pyridin-2-ol, 5-(5-Cyclohexylimino-4-methyl-4,5-dihydro-[1,3,4]thiadiazol-2-yl)-benzene-1,2,3-triol, 2-(5-Cyclohexylimino-4-methyl-4,5-dihydro-[1,3,4]thiadiazol-2-yl)-quinolin-8-ol, Cyclohexyl-(3-methyl-5-pyrazin-2-yl-3H-[1,3,4]thiadiazol-2-ylidene)-amine, 5-[(E)-2-(5-Cyclohexylimino-4-methyl-4,5-dihydro-[1,3,4]thiadiazol-2-yl)-vinyl]-2-methoxy-phenol, 4-(5-Cyclohexylimino-4-methyl-4,5-dihydro-[1,3,4]thiadiazol-2-yl)-2-methoxy-phenol, Cyclohexyl-(3-methyl-5-quinolin-8-yl-3H-[1,3,4]thiadiazol-2-ylidene)-amine, [4-(5-Cyclohexylimino-4-methyl-4,5-dihydro-[1,3,4]thiadiazol-2-yl)-phenyl]-dimethyl-amine, 4-(5-Cyclohexylimino-4-methyl-4,5-dihydro-[1,3,4]thiadiazol-2-yl)-benzenesulfonamide, [5-(5-Chloro-1H-indol-2-yl)-3-methyl-3H-[1,3,4]thiadiazol-2-ylidene]-cyclohexyl-amine; compound with trifluoro-methanesulfonic acid, 2-(5-Cyclohexylimino-4-methyl-4,5-dihydro-[1,3,4]thiadiazol-2-yl)-phenol; compound with 1,1,1-trifluoro-methanesulfonic acid, 5-(5-Cyclohexylimino-4-methyl-4,5-dihydro-[1,3,4]thiadiazol-2-yl)-2-methoxy-phenol, compound with 1,1,1-trifluoro-methanesulfonic acid, 4-(5-Cyclohexylimino-4-methyl-4,5-dihydro-[1,3,4]thiadiazol-2-yl)-phenol, compound with 1,1,1-trifluoro-methanesulfonic acid, Cyclohexyl-[5-(3,4-dimethoxy-phenyl)-3-methyl-3H-[1,3,4]thiadiazol-2-ylidene]-amine, [5-(3-Bromo-4-methoxy-phenyl)-3-methyl-3H-[1,3,4]thiadiazol-2-ylidene]-cyclohexyl-amine, Cyclohexyl-[5-(4-methoxy-phenyl)-3-methyl-3H-[1,3,4]thiadiazol-2-ylidene]-amine, Cyclohexyl-(3-methyl-5-phenyl-3H-[1,3,4]thiadiazol-2-ylidene)-amine, 3-(5-Cyclohexylimino-4-methyl-4,5-dihydro-[1,3,4]thiadiazol-2-yl)-phenol, 4-(5-Cyclohexylimino-4-methyl-4,5-dihydro-[1,3,4]thiadiazol-2-yl)-benzoic acid methyl ester, 4-(5-Cyclohexylimino-4-methyl-4,5-dihydro-[1,3,4]thiadiazol-2-yl)-benzoic acid, 4-(5-Cyclohexylimino-4-methyl-4,5-dihydro-[1,3,4]thiadiazol-2-yl)-N-hydroxy-benzamide, 4-(5-Cyclohexylimino-4-methyl-4,5-dihydro-[1,3,4]thiadiazol-2-yl)-benzamide, 4-(5-Cyclohexylimino-4-methyl-4,5-dihydro-[1,3,4]thiadiazol-2-yl)-N-(2H-tetrazol-5-yl)-benzamide hydrochloride salt, 4-(5-Cyclohexylimino-4-methyl-4,5-dihydro[1,3,4]thiadiazol-2-yl)-N-quinolin-8-yl-benzamide, 4-(5-Cyclohexylimino-4-methyl-4,5-dihydro[1,3,4]thiadiazol-2-yl)-N-(2,6-dimethoxy-pyridin-3-yl)-benzamide, 4-(5-Cyclohexylimino-4-methyl-4,5-dihydro[1,3,4]thiadiazol-2-yl)-N-isopropyl-benzamide, 4-(5-Cyclohexylimino-4-methyl-4,5-dihydro[1,3,4]thiadiazol-2-yl)-N-ethyl-benzamide, Cyclohexyl-{5-[4-(1-ethyl-1H-tetrazol-5-yl)-phenyl]-3-methyl-3H-[1,3,4]thiadiazol-2-ylidene}-amine, 4-(5-Cyclohexylimino-4-methyl-4,5-dihydro[1,3,4]thiadiazol-2-yl)-N-(2-dimethylamino-ethyl)-benzamide, 4-(5-Cyclohexylimino-4-methyl-4,5-dihydro[1,3,4]thiadiazol-2-yl)-N-pyridin-4-ylmethyl-benzamide, 4-(5-Cyclohexylimino-4-methyl-4,5-dihydro-[1,3,4]thiadiazol-2-yl)-N-methyl-N-(1-methyl-piperidin-4-yl)-benzamide, 4-(5-Cyclohexylimino-4-methyl-4,5-dihydro-[1,3,4]thiadiazol-2-yl)-N-isobutyl-benzamide, 4-(5-Cyclohexylimino-4-methyl-4,5-dihydro-[1,3,4]thiadiazol-2-yl)-N-methyl-benzamide, 4-(Cyclohexylimino-methyl-4,5-dihydro-[1,3,4]thiadiazol-2-yl)-N-(2-dimethylamino-ethyl)-N-methyl-benzamide, [4-(5-Cyclohexylimino-4-methyl-4,5-dihydro-[1,3,4]thiadiazol-2-yl)-phenyl]-1-(3-hydroxymethyl-piperidin-1-yl)-methanone, 2-[4-(5-Cyclohexylimino-4-methyl-4,5-dihydro-[1,3,4]thiadiazol-2-yl)-benzoylamino]-3-(4-hydroxy-phenyl)-propionic acid tert-butyl ester, 2-({1-[4-(5-Cyclohexylimino-4-methyl-4,5-dihydro-[1,3,4]thiadiazol-2-yl)-phenyl]-methanoyl}-amino)-3-(4-hydroxy-phenyl)-propionic acid, compound with 2,2,2-trifluoro-acetic acid, (S)-2-[4-(5-Cyclohexylimino-4-methyl-4,5-dihydro-[1,3,4]thiadiazol-2-yl)-benzoylamino]-propionic acid tert-butyl ester, (S)-2-[4-(5-Cyclohexylimino-4-methyl-4,5-dihydro-[1,3,4]thiadiazol-2-yl)-benzoylamino]-propionic acid; compound with 2,2,2-trifluoro-acetic acid, [4-(5-Cyclohexylimino-4-methyl-4,5-dihydro-[1,3,4]thiadiazol-2-yl)-phenyl]-(4-pyridin-2-yl-piperazin-1-yl)-methanone, [4-(5-Cyclohexylimino-4-methyl-4,5-dihydro-[1,3,4]thiadiazol-2-yl)-phenyl]-[4-(4-fluoro-phenyl)-piperazin-1-yl]-methanone, 4-(5-Cyclohexylimino-4-methyl-4,5-dihydro-[1,3,4]thiadiazol-2-yl)-N-(3,4,5-trimethoxy-benzyl)-benzamide, [4-(5-Cyclohexylimino-4-methyl-4,5-dihydro-[1,3,4]thiadiazol-2-yl)-phenyl]-(4-pyrimidin-2-yl-piperazin-1-yl)-methanone, [4-(5-Cyclohexylimino-4-methyl-4,5-dihydro-[1,3,4]thiadiazol-2-yl)-phenyl]-(4-methyl-piperazin-1-yl)-methanone, 4-(5-Cyclohexylimino-4-methyl-4,5-dihydro-[1,3,4]thiadiazol-2-yl)-N-[3-(4-methyl-piperazin-1-yl)-propyl]-benzamide, 4-(5-Cyclohexylimino-4-methyl-4,5-dihydro-[1,3,4]thiadiazol-2-yl)-N-(1-ethyl-pyrrolidin-2-ylmethyl)-benzamide, 4-(5-Cyclohexylimino-4-methyl-4,5-dihydro-[1,3,4]thiadiazol-2-yl)-N-pyridin-3-ylmethyl-benzamide, N-Benzyl-4-(5-cyclohexylimino-4-methyl-4,5-dihydro-[1,3,4]thiadiazol-2-yl)-benzamide, N-(1-Benzyl-piperidin-4-yl)-4-(5-cyclohexylimino-4-methyl-4,5-dihydro-[1,3,4]thiadiazol-2-yl)-benzamide, 4-(5-Cyclohexylimino-4-methyl-4,5-dihydro-[1,3,4]thiadiazol-2-yl)-N-(2-ethyl-2H-pyrazol-3-yl)-benzamide, 4-(5-Cyclohexylimino-4-methyl-4,5-dihydro-[1,3,4]thiadiazol-2-yl)-N-(2-morpholin-4-yl-ethyl)-benzamide, [5-(4-((N-cyano-N′-ethylmorpholine)-carboximidamide)-phenyl)-3-methyl-3H-[1,3,4]thiadiazol-2-ylidene]-cyclohexyl-amine, 4-(5-Cyclohexylimino-4-methyl-4,5-dihydro-[1,3,4]thiadiazol-2-yl)-N-(2-pyrrolidin-1-yl-ethyl)-benzamide, Cyclohexyl-(3-methyl-5-pyridin-3-yl-3H-[1,3,4]thiadiazol-2-ylidene)-amine, 3-(5-Cyclohexylimino-4-methyl-4,5-dihydro-[1,3,4]thiadiazol-2-yl)-benzenesulfonamide, (5-Benzo[1,3]dioxol-5-yl-3-methyl-3H-[1,3,4]thiadiazol-2-ylidene)-cyclohexyl-amine, Cyclohexyl-[3-methyl-5-(3,4,5-trimethoxy-phenyl)-3H-[1,3,4]thiadiazol-2-ylidene]-amine, 4-(5-Cyclopentylimino-4-methyl-4,5-dihydro-[1,3,4]thiadiazol-2-yl)-benzonitrile, 4-(5-Cycloheptylimino-4-methyl-4,5-dihydro-[1,3,4]thiadiazol-2-yl)-benzonitrile, 4-[5-(4-Fluoro-phenylimino)-4-methyl-4,5-dihydro-[1,3,4]thiadiazol-2-yl]-benzonitrile, 4-[5-(3-Hydroxy-phenylimino)-4-methyl-4,5-dihydro-[1,3,4]thiadiazol-2-yl]-benzonitrile, 5-[5-(4-Cyano-phenyl)-3-methyl-3H-[1,3,4]thiadiazol-2-ylideneamino]-2-fluoro-benzoic acid, 4-[4-Methyl-5-(cis-4-methyl-cyclohexylimino)-4,5-dihydro-[1,3,4]thiadiazol-2-yl]-benzonitrile, 4-[4-Methyl-5-(trans-4-methyl-cyclohexylimino)-4,5-dihydro-[1,3,4]thiadiazol-2-yl]-benzonitrile, 4-[5-(trans-4-Hydroxy-cyclohexylimino)-4-methyl-4,5-dihydro-[1,3,4]thiadiazol-2-yl]-benzonitrile, 4-[5-(Bicyclo[2.2.1]hept-2-ylimino)-4-methyl-4,5-dihydro-[1,3,4]thiadiazol-2-yl]-benzonitrile, 4-[5-((1R*,2R*)-2-Hydroxy-cyclohexylimino)-4-methyl-4,5-dihydro-[1,3,4]thiadiazol-2-yl]-benzonitrile, 4-[5-((1R*,2S*)-2-Hydroxy-cyclohexylimino)-4-methyl-4,5-dihydro-[1,3,4]thiadiazol-2-yl]-benzonitrile, 4-[5-((1R*,3R*)-3-Hydroxy-cyclohexylimino)-4-methyl-4,5-dihydro-[1,3,4]thiadiazol-2-yl]-benzonitrile, 4-[5-((1R*,3S*)-3-Hydroxy-cyclohexylimino)-4-methyl-4,5-dihydro-[1,3,4]thiadiazol-2-yl]-benzonitrile, (1R*,3R*))-3-[5-(4-Methanesulfonyl-phenyl)-3-methyl-3H-[1,3,4]thiadiazol-2-ylideneamino]-cyclohexanol, 4-[5-(1R*,3R*)-3-Hydroxy-cyclohexylimino)-4-methyl-4,5-dihydro-[1,3,4]thiadiazol-2-yl]-benzoic acid, 4-[5-((1R*,3R*)-3-hydroxy-cyclohexylimino)-4-methyl-4,5-dihydro-[1,3,4]thiadiazol-2-yl]-N-(2-morpholin-4-yl-ethyl)-benzamide, 4-[5-(trans-4-Hydroxy-cyclohexylimino)-4-methyl-4,5-dihydro-[1,3,4]thiadiazol-2-yl]-benzoic acid, 4-[5-(trans-4-Hydroxy-cyclohexylimino)-4-methyl-4,5-dihydro-[1,3,4]thiadiazol-2-yl]-N-(2-hydroxy-1,1-dimethyl-ethyl)-benzamide, 4-[5-((1R*,3R*)-3-Hydroxy-cyclohexylimino)-4-methyl-4,5-dihydro-[1,3,4]thiadiazol-2-yl]-N-(2-hydroxy-1,1-dimethyl-ethyl)-benzamide, N-tert-Butyl-4-[5-((1R*,3R*)-3-hydroxy-cyclohexylimino)-4-methyl-4,5-dihydro-[1,3,4]thiadiazol-2-yl]-benzamide, N-(1,1-dimethyl-3-oxo-butyl)-4-[5-(1R*,3R*)-3-hydroxy-cyclohexylimino)-4-methyl-4,5-dihydro-[1,3,4]thiadiazol-2-yl]-benzamide, N-(2-Cyano-1,2,2-trimethyl-ethyl)-4-[5-(1R*,3R*)-3-hydroxy-cyclohexylimino)-4-methyl-4,5-dihydro-[1,3,4]thiadiazol-2-yl]-benzamide, 1-{4-[5-((1R*,3R*)-3-Hydroxy-cyclohexylimino)-4-methyl-4,5-dihydro-[1,3,4]thiadiazol-2-yl]-benzoylamino}-cyclopropanecarboxylic acid methyl ester, 4-(5-Cyclopentylimino-4-methyl-4,5-dihydro-[1,3,4]thiadiazol-2-yl)-benzamide, 4-(5-Cycloheptylimino-4-methyl-4,5-dihydro-[1,3,4]thiadiazol-2-yl)-benzamide, 4-[5-(4-Fluoro-phenylimino)-4-methyl-4,5-dihydro-[1,3,4]thiadiazol-2-yl]-benzamide, 4-[5-(3-Hydroxy-phenylimino)-4-methyl-4,5-dihydro-[1,3,4]thiadiazol-2-yl]-benzamide, 5-[5-(4-Carbamoyl-phenyl)-3-methyl-3H-[1,3,4]thiadiazol-2-ylideneamino]-2-fluoro-benzoic acid, 4-[4-Methyl-5-(4-methyl-cyclohexylimino)-4,5-dihydro-[1,3,4]thiadiazol-2-yl]-benzamide, 4-[5-(4-Hydroxy-cyclohexylimino)-4-methyl-4,5-dihydro-[1,3,4]thiadiazol-2-yl]-benzamide, 4-[5-(Bicyclo[2.2.1]hept-2-ylimino)-4-methyl-4,5-dihydro-[1,3,4]thiadiazol-2-yl]-benzamide, 4-[5-((1R*,2R*)-2-Hydroxy-cyclohexylimino)-4-methyl-4,5-dihydro-[1,3,4]thiadiazol-2-yl]-benzamide, 4-[5-((1R*,2S*)-2-Hydroxy-cyclohexylimino)-4-methyl-4,5-dihydro-[1,3,4]thiadiazol-2-yl]-benzamide, 4-[5-((1R*,3R*)-3-Hydroxy-cyclohexylimino)-4-methyl-4,5-dihydro-[1,3,4]thiadiazol-2-yl]-benzamide, 4-[5-((1R*,3S*)-3-Hydroxy-cyclohexylimino)-4-methyl-4,5-dihydro-[1,3,4]thiadiazol-2-yl]-benzamide, 4-[4-Methyl-5-(3-oxo-cyclohexylimino)-4,5-dihydro-[1,3,4]thiadiazol-2-yl]-benzamide, 4-[5-(3,3-Difluoro-cyclohexylimino)-4-methyl-4,5-dihydro-[1,3,4]thiadiazol-2-yl]-benzamide, 4-[5-((1R*,3R*)-3-Fluoro-cyclohexylimino)-4-methyl-4,5-dihydro-[1,3,4]thiadiazol-2-yl]-benzamide, 4-[5-(Cyclohex-3-enylimino)-4-methyl-4,5-dihydro-[1,3,4]thiadiazol-2-yl]-benzamide, (1R*,3R*)-3-{3-Methyl-5-[4-(1H-tetrazol-5-yl)-phenyl]-3H-[1,3,4]thiadiazol-2-ylideneamino}-cyclohexanol, 3-[5-(4-Chloro-phenyl)-3-methyl-3H-[1,3,4]thiadiazol-2-ylideneamino]-2-hydroxy-benzoic acid, 3-[5-(4-Cyano-phenyl)-3-methyl-3H-[1,3,4]thiadiazol-2-ylideneamino]-benzoic acid, 3-[5-(4-carbamoyl-phenyl)-3-methyl-3H-[1,3,4]thiadiazol-2-ylideneamino]-benzoic acid, 2-Fluoro-5-[5-(4-methanesulfonyl-phenyl)-3-methyl-3H-[1,3,4]thiadiazol-2-ylideneamino]-benzoic acid, 3-[5-(4-methanesulfonyl-phenyl)-3-methyl-3H-[1,3,4]thiadiazol-2-ylideneamino]-cyclohexanecarboxylic acid, [5-(4-methanesulfonyl-phenyl)-3-methyl-3H-[1,3,4]thiadiazol-2-ylidene]-piperidin-1-yl amine, [5-(4-Methanesulfonyl-phenyl)-3-methyl-3H-[1,3,4]thiadiazol-2-ylidene]-(tetrahydro-pyran-4-yl)-amine, 3-[5-(4-Acetylamino-phenyl)-3-methyl-3H-[1,3,4]thiadiazol-2-ylideneamino]-benzoic acid, N-{4-[5-(trans-4-Hydroxy-cyclohexylimino)-4-methyl-4,5-dihydro-[1,3,4]thiadiazol-2-yl]-phenyl}-acetamide, N-{4-[5-((1R*,3S*)-3-Hydroxy-cyclohexylimino)-4-methyl-4,5-dihydro-[1,3,4]thiadiazol-2-yl]-phenyl}-acetamide, N-{4-[5-((1R*,3R*)-3-Hydroxy-cyclohexylimino)-4-methyl-4,5-dihydro-[1,3,4]thiadiazol-2-yl]-phenyl}-acetamide, N-{5-[5-((1R*,3R*)-3-Hydroxy-cyclohexylimino)-4-methyl-4,5-dihydro-[1,3,4]thiadiazol-2-yl]-pyridin-2-yl}-acetamide, 3-[5-(4-Chloro-phenyl)-3-methyl-3H-[1,3,4]thiadiazol-2-ylideneamino]-benzonitrile, [5-(4-Chloro-phenyl)-3-methyl-3H-[1,3,4]thiadiazol-2-ylidene]-[3-(1H-tetrazol-5-yl)-phenyl]-amine, 3-[5-(4-Chloro-phenyl)-3-methyl-3H-[1,3,4]thiadiazol-2-ylideneamino]-N-hydroxy-benzamidine, 3-{3-[5-(4-Chloro-phenyl)-3-methyl-3H-[1,3,4]thiadiazol-2-ylideneamino]-phenyl}-[1,2,4]oxadiazol-5-ol, [5-(4-Bromo-3-methyl-phenyl)-3-methyl-3H-[1,3,4]thiadiazol-2-ylidene]-cyclohexyl-amine, 4-(5-Cyclohexylimino-4-methyl-4,5-dihydro-[1,3,4]thiadiazol-2-yl)-2-methyl-benzonitrile, 4-(5-Cyclohexylimino-4-methyl-4,5-dihydro-[1,3,4]thiadiazol-2-yl)-2-methyl-benzamide, [5-(4-Bromo-3-methoxy-phenyl)-3-methyl-2,3-dihydro-[1,3,4]thiadiazol-2-yl]-cyclohexyl-amine, 4-(5-Cyclohexylimino-4-methyl-4,5-dihydro-[1,3,4]thiadiazol-2-yl)-2-methoxy-benzamide, 4-(5-Cyclohexylimino-4-methyl-4,5-dihydro-[1,3,4]thiadiazol-2-yl)-2-hydroxy-benzamide, 4-(5-Cyclohexylimino-4-methyl-4,5-dihydro-[1,3,4]thiadiazol-2-yl)-2-nitro-benzoic acid methyl ester, 2-Amino-4-(5-cyclohexylimino-4-methyl-4,5-dihydro-[1,3,4]thiadiazol-2-yl)-benzoic acid methyl ester, 2-Acetylamino-4-(5-cyclohexylimino-4-methyl-4,5-dihydro-[1,3,4]thiadiazol-2-yl)-benzoic acid methyl ester, 2-Amino-4-(5-cyclohexylimino-4-methyl-4,5-dihydro-[1,3,4]thiadiazol-2-yl)-benzamide, 7-(5-Cyclohexylimino-4-methyl-4,5-dihydro-[1,3,4]thiadiazol-2-yl)-3H-quinazolin-4-one, 7-(5-Cyclohexylimino-4-methyl-4,5-dihydro-[1,3,4]thiadiazol-2-yl)-quinazolin-4-ylamine, 7-(5-Cyclohexylimino-4-methyl-4,5-dihydro-[1,3,4]thiadiazol-2-yl)-1H-quinazoline-2,4-dione, 4-(5-Cyclohexylimino-4-methyl-4,5-dihydro-[1,3,4]thiadiazol-2-yl)-2-methoxy-benzenesulfonamide, 5-(5-Cyclohexylimino-4-methyl-4,5-dihydro-[1,3,4]thiadiazol-2-yl)-2-methoxy-benzenesulfonamide, 3-[5-(3-Cyano-phenyl)-3-methyl-3H-[1,3,4]thiadiazol-2-ylideneamino]-benzoic acid methyl ester, 3-[5-(3-Cyano-phenyl)-3-methyl-3H-[1,3,4]thiadiazol-2-ylideneamino]-benzoic acid, 3-[3-Methyl-5-pyridin-2-yl-3H-[1,3,4]thiadiazol-2-ylideneamino]-benzoic acid, 3-[5-(4-Chloro-3-sulfamoyl-phenyl)-3-methyl-3H-[1,3,4]thiadiazol-2-ylideneamino]-benzoic acid, 4-(5-Cyclohexylimino-4-methyl-4,5-dihydro-[1,3,4]thiadiazol-2-yl)-benzonitrile, Cyclohexyl-{3-methyl-5-[4-(1H-tetrazol-5-yl)-phenyl]-3H-[1,3,4]thiadiazol-2-ylidene}-amine, Cyclohexyl-[3-methyl-5-(4-nitro-phenyl)-3H-[1,3,4]thiadiazol-2-ylidene]-amine, 4-(5-Cyclohexylimino-4-methyl-4,5-dihydro-[1,3,4]thiadiazol-2-yl)-phenylamine, [5-(4-(N-cyano-N′-(2-dimethylaminoethyl)-carboximidamide)-phenyl)-3-methyl-3H-[1,3,4]thiadiazol-2-ylidene]-cyclohexyl-amine, N-[4-(5-Cyclohexylimino-4-methyl-4,5-dihydro-[1,3,4]thiadiazol-2-yl)-phenyl]-acetamide, [5-(4-(bis-ethylsulfonylamino)-phenyl)-3-methyl-3H-[1,3,4]thiadiazol-2-ylidene]-cyclohexyl-amine, [5-(4-(1-(2-dimethylaminoethyl)amino-2-nitro-vinylamino)-phenyl)-3-methyl-3H-[1,3,4]thiadiazol-2-ylidene]-cyclohexyl-amine, (E)-N¹-[4-(5-Cyclohexylimino-4-methyl-4,5-dihydro-[1,3,4]thiadiazol-2-yl)-phenyl]-2-nitro-ethene-1,1-diamine, [5-(N-cyano-N′-methyl-4-carboximidamide-phenyl)-3-methyl-3H-[1,3,4]thiadiazol-2-ylidene]-cyclohexyl-amine, [5-(4-(N-cyano-N′-amino-carboximidamide)-phenyl)-3-methyl-3H-[1,3,4]thiadiazol-2-ylidene]-cyclohexyl-amine, Ethanesulfonic acid [4-(5-cyclohexylimino-4-methyl-4,5-dihydro-[1,3,4]thiadiazol-2-yl)-phenyl]-amide, [4-(5-Cyclohexylimino-4-methyl-4,5-dihydro-[1,3,4]thiadiazol-2-yl)-phenyl]-urea, 1-[4-(Cyclohexylimino-methyl-4,5-dihydro-[1,3,4]thiadiazol-2-yl)-phenyl]-3-(2-dimethylamino-ethyl)-urea, 2-Chloro-4-(5-cyclohexylimino-4-methyl-4,5-dihydro-[1,3,4]thiadiazol-2-yl)-benzenesulfonamide, 2-Chloro-4-(5-cyclohexylimino-4-methyl-4,5-dihydro-[1,3,4]thiadiazol-2-yl)-benzoic acid methyl ester, 2-Chloro-4-(5-cyclohexylimino-4-methyl-4,5-dihydro-[1,3,4]thiadiazol-2-yl)-benzamide, 2-Chloro-5-(5-cyclohexylimino-4-methyl-4,5-dihydro-[1,3,4]thiadiazol-2-yl)-benzamide, 4-(5-Cyclohexylimino-4-methyl-4,5-dihydro-[1,3,4]oxadiazol-2-yl)-benzoic acid methyl ester, and, 4-(5-Cyclohexylimino-4-methyl-4,5-dihydro-[1,3,4]oxadiazol-2-yl)-benzamide.

Of the compounds of formula (VI) disclosed in WO02/28847, further preferred are compounds selected from the group consisting of:
5-(5-Cyclohexylimino-4-methyl-4,5-dihydro[1,3,4]thiadiazol-2-yl)-2-methoxy-benzene-1,3-diol; compound with trifluoro-methanesulfonic acid, 5-(5-Cyclohexylimino-4-methyl-4,5-dihydro[1,3,4]thiadiazol-2-yl)-2,3-dimethoxy-phenol; compound with trifluoro-methanesulfonic acid, 2-Chloro-5-(5-cyclohexylimino-4-methyl-4,5-dihydro-[1,3,4]thiadiazol-2-yl)-benzenesulfonamide, 2-Chloro-5-(5-cyclohexylimino-4-methyl-4,5-dihydro[1,3,4]thiadiazol-2-yl)-N,N-diethyl-benzenesulfonamide, {5-[4-Chloro-3-(4-methyl-piperazine-1-sulfonyl)-phenyl]-3-methyl-3H-[1,3,4]thiadiazol-2-ylidene}-cyclohexyl-amine, 2-Chloro-5-(5-cyclohexylimino-4-methyl-4,5-dihydro-[1,3,4]thiadiazol-2-yl)-N-pyridin-4-ylmethyl-benzenesulfonamide, 2-Chloro-5-(5-cyclohexylimino-4-methyl-4,5-dihydro-[1,3,4]thiadiazol-2-yl)-N-(2-morpholin-4-yl-ethyl)-benzenesulfonamide, 2-Chloro-5-(5-cyclohexylimino-4-methyl-4,5-dihydro-[1,3,4]thiadiazol-2-yl)-N-ethyl-benzenesulfonamide, 2-Chloro-5-(5-cyclohexylimino-4-methyl-4,5-dihydro-[1,3,4]thiadiazol-2-yl)-N-ethyl-N-(2-morpholin-4-yl-ethyl)-benzenesulfonamide, 2-Chloro-5-(5-cyclohexylimino-4-methyl-4,5-dihydro-[1,3,4]thiadiazol-2-yl)-N-isopropyl-N-(2-morpholin-4-yl-ethyl)-benzenesulfonamide, 2-Chloro-5-(5-cyclohexylimino-4-methyl-4,5-dihydro-[1,3,4]thiadiazol-2-yl)-N-ethyl-N-[2-(2-methoxy-ethoxy)-ethyl]-benzenesulfonamide, 2-Chloro-5-(cyclohexylimino-methyl-4,5-dihydro-[1,3,4]thiadiazol-2-yl)-N-(3-dimethylamino-2-hydroxy-propyl)-N-ethyl-benzenesulfonamide, 2-Chloro-5-(5-cyclohexylimino-4-methyl-4,5-dihydro-[1,3,4]thiadiazol-2-yl)-N-(2,3-dihydroxy-propyl)-N-ethyl-benzenesulfonamide, 2-Chloro-5-(5-cyclohexylimino-4-methyl-4,5-dihydro-[1,3,4]thiadiazol-2-yl)-N-ethyl-N-(2-hydroxy-3-pyrrolidin-1-yl-propyl)-benzenesulfonamide, 3-(5-Cyclohexylimino-4-methyl-4,5-dihydro-[1,3,4]thiadiazol-2-yl)-benzamide, 4-(5-Cyclohexylimino-4-methyl-4,5-dihydro-[1,3,4]thiadiazol-2-yl)-benzamide, 4-(5-Cyclohexylimino-4-methyl-4,5-dihydro[1,3,4]thiadiazol-2-yl)-N-quinolin-8-yl-benzamide, 4-(5-Cyclohexylimino-4-methyl-4,5-dihydro[1,3,4]thiadiazol-2-yl)-N-(2,6-dimethoxy-pyridin-3-yl)-benzamide, 4-(5-Cyclohexylimino-4-methyl-4,5-dihydro[1,3,4]thiadiazol-2-yl)-N-isopropyl-benzamide, 4-(5-Cyclohexylimino-4-methyl-4,5-dihydro[1,3,4]thiadiazol-2-yl)-N-ethyl-benzamide, 4-(5-Cyclohexylimino-4-methyl-4,5-dihydro[1,3,4]thiadiazol-2-yl)-N-(2-dimethylamino-ethyl)-benzamide, 4-(5-Cyclohexylimino-4-methyl-4,5-dihydro[1,3,4]thiadiazol-2-yl)-N-pyridin-4-ylmethyl-benzamide, 4-(5-Cyclohexylimino-4-methyl-4,5-dihydro-[1,3,4]thiadiazol-2-yl)-N-methyl-N-(1-methyl-piperidin-4-yl)-benzamide, 4-(5-Cyclohexylimino-4-methyl-4,5-dihydro-[1,3,4]thiadiazol-2-yl)-N-methyl-benzamide, 2-[4-(5-Cyclohexylimino-4-methyl-4,5-dihydro-[1,3,4]thiadiazol-2-yl)-benzoylamino]-3-(4-hydroxy-phenyl)-propionic acid tert-butyl ester, (S)-2-[4-(5-Cyclohexylimino-4-methyl-4,5-dihydro-[1,3,4]thiadiazol-2-yl)-benzoylamino]-3-(4-hydroxy-phenyl)-propionic acid; compound with 2,2,2-trifluoro-acetic acid, 4-(5-Cyclohexylimino-4-methyl-4,5-dihydro-[1,3,4]thiadiazol-2-yl)-N-(3,4,5-trimethoxy-benzyl)-benzamide, 4-(5-Cyclohexylimino-4-methyl-4,5-dihydro-[1,3,4]thiadiazol-2-yl)-N-[3-(4-methyl-piperazin-1-yl)-propyl]-benzamide, 4-(5-Cyclohexylimino-4-methyl-4,5-dihydro-[1,3,4]thiadiazol-2-yl)-N-pyridin-3-ylmethyl-benzamide, N-(1-Benzyl-piperidin-4-yl)-4-(5-cyclohexylimino-4-methyl-4,5-dihydro-[1,3,4]thiadiazol-2-yl)-benzamide, 4-(5-Cyclohexylimino-4-methyl-4,5-dihydro-[1,3,4]thiadiazol-2-yl)-N-(2-ethyl-2H-pyrazol-3-yl)-benzamide, 4-(5-Cyclohexylimino-4-methyl-4,5-dihydro-[1,3,4]thiadiazol-2-yl)-N-(2-morpholin-4-yl-ethyl)-benzamide, 4-(5-Cyclohexylimino-4-methyl-4,5-dihydro-[1,3,4]thiadiazol-2-yl)-N-(2-pyrrolidin-1-yl-ethyl)-benzamide, 3-[5-(4-carbamoyl-phenyl)-3-methyl-3H-[1,3,4]thiadiazol-2-ylideneamino]-benzoic acid, [5-(4-Chloro-phenyl)-3-methyl-3H-[1,3,4]thiadiazol-2-ylidene]-[3-(1H-tetrazol-5-yl)-phenyl]-amine, 2-Amino-4-(5-cyclohexylimino-4-methyl-4,5-dihydro-[1,3,4]thiadiazol-2-yl)-benzoic acid methyl ester, 2-Amino-4-(5-cyclohexylimino-4-methyl-4,5-dihydro-[1,3,4]thiadiazol-2-yl)-benzamide, 7-(5-Cyclohexylimino-4-methyl-4,5-dihydro-[1,3,4]thiadiazol-2-yl)-3H-quinazolin-4-one, 7-(5-Cyclohexylimino-4-methyl-4,5-dihydro-[1,3,4]thiadiazol-2-yl)-quinazolin-4-ylamine, N-[4-(5-Cyclohexylimino-4-methyl-4,5-dihydro-[1,3,4]thiadiazol-2-yl)-phenyl]-acetamide, and, 1-[4-(Cyclohexylimino-methyl-4,5-dihydro-[1,3,4]thiadiazol-2-yl)-phenyl]-3-(2-dimethylamino-ethyl)-urea.

Further examples of suitable PDE7 inhibitors for use in the invention include those compounds generally or specifically disclosed in the published patent application WO 03/082277. Particularly preferred are N-{4-[(2Z)-2-(cyclohexylimino)-3-methyl-2,3-dihydro-1,3-thiazol-5-yl]phenyl}acetamide, N-{4-[(2Z)-2-[(3-hydroxycyclohexyl)imino]-3-methyl-2,3-dihydro-1,3-thiazol-5-yl]phenyl}acetamide, 7-[(2Z)-2-(cyclohexylimino)-3-methyl-2,3-dihydro-1,3-thiazol-5-yl]quinazolin-4-amine, and 7-{(2Z)-2-[(3-hydroxycyclohexyl)imino]-3-methyl-2,3-dihydro-1,3-thiazol-5-yl}quinazolin-4-amine, optionally its racemics forms, its isomers, and its pharmaceutically acceptable acid or base salts.

Further examples of suitable PDE7 inhibitors for use in the invention include those compounds generally or specifically disclosed in the published patent application WO 03/082839. Particularly preferred are N-{4-[5-(cyclohexylamino)-4-methyl-1,3-thiazol-2-yl]phenyl}acetamide, N-{4-[5-[(3-hydroxycyclohexyl)amino]-4-methyl-1,3-thiazol-2-yl]phenyl}acetamide, 7-[5-(cyclohexylamino)-4-methyl-1,3-thiazol-2-yl]quinazolin-4-amine, and 7-{5-[(3-hydroxycyclohexyl)amino]-4-methyl-1,3-thiazol-2-yl}quinazolin-4-amine, optionally its racemics forms, its isomers, and its pharmaceutically acceptable acid or base salts.

Examples of suitable PDE7 inhibitors for use in the invention include those compounds generally or specifically disclosed in the publication of A. Castro, M. I. Abasolo, C. Gil, V. Segarra and A. Martinez. Eur. J. Med. Chem. 36 (2001), pp. 333-338 in particular the compounds which are benzyl derivatives of 2,1,3-benzo[3,2-a]thiadiazine 2,2-dioxides and 2,1,3-benzothieno[3,2-a]thiadiazine 2,2-dioxides and pharmaceutically acceptable salts and solvates thereof.

Further examples of suitable PDE7 inhibitors for use in the invention include those compounds generally or specifically disclosed in the publication of Barnes Mj, Cooper N, Davenport R J, Biorg. Med. Chem. Lett. (2001) 23 (8): 1081-1083, 338 in particular the compounds which are guanine analogues, the 8-bromo-9-substituted compounds being the most preferred, and pharmaceutically acceptable salts and solvates thereof.

Further examples of suitable PDE7 inhibitors for use in the invention include those compounds generally or specifically disclosed in the publication of Pitts, W J., et al Biorg. Med. Chem. Left 14 2004 2955-2958, particularly the compounds which are purine based compounds and pharmaceutically acceptable salts and solvates thereof.

Further examples of suitable PDE7 inhibitors for use in the invention include those compounds generally or specifically disclosed in the publication of lorthiois, E., et al Biorg. Med. Chem. Lett, 14 2004 4623-4626 particularly the compounds which are spiroquinazolinones and pharmaceutically acceptable salts and solvates thereof.

Further examples of suitable PDE7 inhibitors for use in the invention include those compounds generally or specifically disclosed in the publication of Bernardelli, P., et al Bioorg. Med. Chem. Lett, 14 2004 4627-4631, particularly the compounds which are 5,8-disubstituted spirocyclohexane-quinazolinones particularly 5 substituted 8-chloro-spirocyclohexane-quinazolinones derivatives such as 5-alkoxy-8 chloro-quinazolinone, and pharmaceutically acceptable salts and solvates thereof.

Further examples of suitable PDE7 inhibitors for use in the invention include those compounds generally or specifically disclosed in the publication of Vergne, F., et al Bioorg. Med. Chem. Lett, 2004, 14, 4607-461 & Vergne, F., et al Bioorg. Med. Chem. Lett, 2004, 14, 4615-4621, particularly the compounds which are thiadiazoles and pharmaceutically acceptable salts and solvates thereof.

Further examples of suitable PDE7 inhibitors for use in the invention include those compounds generally or specifically disclosed in the patent application WO0198274 (CellTech Chiroscience Ltd), M-substituted phenyl-N-phenylsulfonamides particularly N-phenyl-3-benzoxazol-2-ylphenylsulfonamide and N-phenyl-3-benzimidazol-2-ylphenylsulfonamide derivatives.

Patent application WO 0198274 (Celltech Chiroscience) discloses further examples of suitable PDE7 inhibitors which are sulfonamides and suitable for use in the invention.

In addition, patent application WO0174786 (Darwin Discovery Ltd) discloses further examples of PDE7 inhibitors suitable for use in the invention and which are a series of heterobiarylsulphonamides particularly suitable are the N-aryl-3-benzimidazolylbenzenesulfonamides. Patent application WO0068230 (Darwin Discovery Ltd) discloses further suitable PDE7 inhibitors, 9-(1,2,3,4-Tetrahydronapthalen-1-yl)-1,9-dihydropurin-6-one derivatives also published in, Bioorganic and Medicinal Chemistry Letters 2001, 1081-1083.

Patent applications WO0129049 (Merck), WO0136425 (Merck) and DE 19954707 (Merck) disclose imidazole derivatives, WO0132175 (Merck) and DE 19953024 (Merck) disclose isoxazole derivatives, WO0132618 (Merck) and DE 19953025 (Merck) disclose pyrrole derivatives, DE19953414 (Merck) discloses imidazo[4,5-c]pyridine derivatives, all of which are further examples of PDE7 inhibitors and suitable for use in the invention.

Further examples of suitable PDE7 inhibitors include antibodies or antibody subdomains to PDE7, particularly anti PDE7 monoclonal antibody or antibody subdomains for example an antibody or subdomain specific for PDE7, or an antibody or subdomain specific for an epitope provided in part by cAMP or AMP.

Further examples of suitable PDE7 inhibitors suitable for use in the invention include those compounds generally or specifically disclosed in the following patent applications:

WO2004111054 which discloses (Pyridinyl)pyrazolopyrimidinones (Daichi Suntory) as PDE7 inhibitors.

WO03053975 which discloses Pyrazolopyrimidinones (Daiichi Suntory) as PDE7 inhibitors.

WO 2004111053 which discloses Imidazotriazinones (Daichi Suntory) as PDE7 inhibitors.

WO02102314 which discloses Purine Inhibitors (Bristol-Myers-Squibb) as PDE7 inhibitors, also disclosed in the literature reference Biorganic and Medicinal Chemistry Letters 2004, 14, 2955-2958.

WO02102315 which discloses Quinazoline and pyrido[2,3-d]pyrimidines (Bristol-Myers-Squibb) as PDE7 inhibitors.

WO02102313 which discloses Pyrimidines (Bristol-Myers-Squibb) as PDE7 inhibitors.

WO02088079 and WO02088080 which disclose related structures described as mixed PDE4/7 inhibitors.

US 2002-683897 which discloses BRL 50481 (Smithkline Beecham) as a PDE7 inhibitors which is also disclosed in the publication, Molecular Pharmacology (2004), 66(6), 1679-1689.

WO2004065391 which discloses 4-aminothieno[2,3-d]pyrimidine-6-carbonitrile derivatives (Almirall Prodesfarma S.A) as PDE7 inhibitors.

WO03064389 which discloses Isoquinolines (Ono Pharmaceutical Co) as PDE7 inhibitors.

WO03057149 which discloses Fused pyrimidines (Bayer) as PDE7 inhibitors.

US2003119829 which discloses 4-amino-5,6-substituted thiopheno[2,3-d]pyrimidines for use in the treatment or prevention of PDE7B mediated diseases (Bayer) as PDE7 inhibitors.

WO02085906 which discloses Phthalazinones as PDE4/7 inhibitors (Altana Pharma) as PDE7 inhibitors.

WO02085894 which discloses Arylindenopyridines as PDE7 inhibitors (Ortho-McNeil Pharmaceuticals).

WO0240450 which discloses (Dihydro)isoquinolines as phosphodiesterase inhibitors (BYK Gulden Lomberg Chemische Fabrik) as PDE7 inhibitors.

Preferably a PDE7 inhibitor according to the present invention is centrally acting. In order to be centrally acting such a compound should be able to penetrate the blood brain barrier.

### DEFINITIONS

In the compounds of Formulae (I), (II) and (III) disclosed in WO 02/074754, the groups are defined as follows:

Halogen includes fluoro, chloro, bromo, and iodo. Preferred halogens are F and Cl.

Lower alkyl includes straight and branched carbon chains having from 1 to 6 carbon atoms. Examples of such alkyl groups include methyl, ethyl, isopropyl, tert-butyl and the like.

Lower alkenyl includes straight and branched hydrocarbon radicals having from 2 to 6 carbon atoms and at least one double bond. Examples of such alkenyl groups are ethenyl, 3-buten-1-yl, 2-ethenylbutyl, 3-hexen-1-yl, and the like.

Lower alkynyl includes straight and branched hydrocarbon radicals having from 2 to 6 carbon atoms and at least one triple bond. Examples of such alkynyl groups are ethynyl, 3-butyn-1-yl, propynyl, 2-butyn-1-yl, 3-pentyn-1-yl, and the like.

Lower haloalkyl includes a lower alkyl as defined above, substituted with one or several halogens. A preferred haloalkyl is trifluoromethyl.

Aryl is understood to refer to an aromatic carbocycle containing between 6 and 10, preferably 6, carbon atoms. A preferred aryl group is phenyl.

Heteroaryl includes aromatic cycles which have from 5 to 10 ring atoms, from 1 to 4 of which are independently selected from the group consisting of O, S, and N. Preferred heteroaryl groups have 1, 2, 3 or 4 heteroatoms in a 5- or 6-membered aromatic ring. Examples of such groups are tetrazole, pyridyl, thienyl and the like.

Preferred cycloalkyl contain from 3 to 8 carbon atoms. Examples of such groups are cyclopropyl, cyclobutyl, cyclopentyl, cyclohexyl, cycloheptyl and cyclooctyl.

The term “interrupted” means that in a backbone chain, a carbon atom is replaced by an heteroatom or a group as defined herein. For example, in “cycloalkyl or cycloalkenyl optionally interrupted with C(═O) or with 1 heteroatom chosen from O, S, S(═O), SO₂ or N”, the term “interrupted” means that C(═O) or a heteroatom can replace a carbon atom of the ring. Example of such groups are morpholine or piperazine.

Cycloalkenyl includes 3- to 10-membered cycloalkyl containing at least one double bond.

Heterocyclic ring include heteroaryl as defined above and cycloalkyl or cycloalkenyl, as defined above, interrupted with 1, 2 or 3 heteroatoms chosen from O, S, S(═O), SO₂, or N.

Bicyclic substituents refer to two cycles, which are the same or different and which are chosen from aryl, heterocyclic ring, cycloalkyl or cycloalkenyl, fused together to form said bicyclic substituents. A preferred bicyclic substituent is indolyl.

Sp² hybridization state: carbon atoms in an sp² hybridization state are trigonal instead of tetraedric. It means that the carbon atoms in a sp² hybridization state are linked to three atoms and form a double bond with one of these three atoms.
aryl is understood to refer to an unsaturated carbocycle, exclusively comprising carbon atoms in the cyclic structure, the number of which is between 5 and 10, including phenyl, naphthyl or tetrahydronaphthyl;
heterocycle is understood to refer to a non-saturated or saturated monocycle containing between 1 and 7 carbon atoms in the cyclic structure and at least one heteroatom in the cyclic structure, such as nitrogen, oxygen, or sulfur, preferably from 1 to 4 heteroatoms, identical or different, selected from nitrogen, sulfur and oxygen atoms. Suitable heterocycles include morpholinyl, piperazinyl, pyrrolidinyl, piperidinyl, pyrimidinyl, 2- and 3-furanyl, 2- and 3-thienyl, 2-pyridyl, 2- and 3-pyranyl, hydroxypyridyl, pyrazolyl, isoxazolyl, tetrazole, imidazole, triazole and the like;
polycyclic groups include at least two cycles, identical or different, selected from aryl, heterocycle, cycloalkyl, cycloalkenyl groups fused together to form said polycyclic group such as 2- and 3-benzothienyl, 2- and 3-benzofuranyl, 2-indolyl, 2- and 3-quinolinyl, acridinyl, quinazolinyl, indolyl benzo[1,3]dioxolyl and 9-thioxantanyl. Preferred polycyclic groups include 2 or 3 cycles as defined above. More preferred polycyclic groups include 2 cycles (bicyclic substituents) as defined above-bicyclic groups refer to two cycles, which are the same or different and which are chosen from aryl, heterocycle, cycloalkyl or cycloalkenyl, fused together to form said bicyclic groups;

In the compounds of formula (IV) disclosed in U.S. 60/741,854 the groups are defined as follows:

the term “alkylene” denotes a divalent saturated hydrocarbon chain having 1 or 2 carbon atoms. Examples of alkylene groups include methylene, ethylene and methylmethylene, of which methylene is preferred.

The term “cycloalkylene” denotes a divalent saturated carbocyclic ring having 3 to 6 carbon atoms. Examples of cycloalkylene groups include cyclopropylene (eg 1,1-cyclopropylene and cis- and trans-1,2-cyclopropylene), cyclobutylene (eg 1,1-cyclobutylene, cis- and trans-1,2-cyclobutylene, and cis- and trans-1,3-cyclobutylene), cyclopentylene (eg 1,1-cyclopentylene, cis- and trans-1,2-cyclopentylene, and cis- and trans-1,3-cyclopentylene) and cyclohexylene (eg 1,1-cyclohexylene, cis- and trans-1,2-cyclohexylene, cis- and trans-1,3-cyclohexylene) and cis- and trans-1,4-cyclohexylene). Preferred examples include cyclobutylene and cyclohexylene, more preferably cyclobutylene, even more preferably 1,3-cyclobutylene, and most preferably trans-1,3-cyclobutylene.

The term “alkyl” denotes a monovalent, straight or branched, saturated hydrocarbon chain containing 1 to 4 carbon atoms. Examples of alkyl groups include methyl, ethyl, n-propyl, isopropyl, n-butyl, isobutyl, sec-butyl and tert-butyl. Preferred examples include methyl and ethyl, especially methyl.

The cycloalkylene group is optionally substituted with a C₁₋₄ alkyl group. Preferably, the alkyl substituent, if present, is a methyl or ethyl group, more preferably a methyl group. The alkyl substituent, if present, may be present at any position on the ring, but is preferably present at the 1-position (ie the same position as the carboxylic acid group).

In the compounds of formula (V) disclosed in WO 04/026818 the groups are defined as follows:

The term “linear or branched (C₁-C₆)alkylene group” represent a carbon atom chain, linear or branched containing from 1 to 6 carbon atoms. Examples of such (C₁-C₆)alkylene are methylene, ethylene, isopropylene, tert-butylene and the like.

The term “(C₁-C₆)alkyl” represent a linear or branched carbon atom chain containing from 1 to 6 carbon atoms. Example of “(C₁-C₆)alkyl” are methyl, ethyl, propyl, butyl, isopropyl, tert-butyl and the like.

Examples of “saturated 4 to 6-membered heterocycle comprising one or two heteroatoms selected from nitrogen or oxygen” are azetidine, pyrrolidine, piperidine, tetrahydrofurane, tetrahydropyrane, morpholine and piperazine.

A preferred “saturated 4 to 6-membered heterocycle comprising a nitrogen atom or an oxygen atom” is azetidine.

Examples of “4 to 8-membered, aromatic or non aromatic, heterocycle comprising 1 to 4 heteroatoms selected from O, S, S(═O), SO₂ and N” are isoxazolyl, oxazolyl, thiazolyl, isothiazolyl, triazolyl, oxadiazolyl, thiadiazolyl, pyridyl, pyrazolyl, imidazolyl, azetidine, pyrrolidine, piperidine, tetrahydrofurane, tetrahydropyrane, morpholine and piperazine. Preferably, said heterocycle is 5 or 6-membered, aromatic, and comprises 1 or 2 nitrogen atoms. Examples of such groups are pyridyl, pyrazolyl and imidazolyl.

In the compounds of Formula (VI) disclosed in WO 02/28847 the groups are defined as follows:
halogen is understood to refer to fluorine, chlorine, bromine or iodine;
lower alkyl is understood to mean that the alkyl is linear or branched and contains 1 to 6 carbon atoms; Examples of lower alkyl groups include methyl, ethyl, propyl, butyl, isopropyl, tert-butyl, isobutyl, n-butyl, pentyl, hexyl and the like.
alkenyl is understood to refer to a linear or branched unsaturated carbon atom chain, comprising one or several double bonds, preferably one or two double bonds. Preferred alkenyls comprise from 3 to 6 carbon atoms and one double bonds.
alkynyl is understood to refer to a linear or branched unsaturated carbon atom chain, comprising one or several triple bonds, preferably one or two triple bonds. Preferred alkynyls comprise from 3 to 6 carbon atoms and one triple bonds.
lower haloalkyl are understood to refer to a lower alkyl substituted with one or several halogens; Preferred lower haloalkyl groups include perhaloalkyl groups such as CF₃.
cycloalkyl is understood to refer to saturated monocarbocyle containing from 3 to 10 carbon atoms; preferred cycloalkyl groups comprise cyclopropyl, cyclobutyl, cyclopentyl, cyclohexyl and cycloheptyl.
cycloalkenyl is understood to refer to unsaturated monocarbocyle containing from 3 to 10 carbon atoms. Preferred cycloalkenyl groups contain 1 or 2 double bonds. Examples of suitable cycloalkenyl are 3-cyclohexene, 3-cycloheptene or the like.
carboxylic acid bioisostere has the classical meaning; common carboxylic acid bioisostere, are tetrazol, hydroxamic acid, isoxazole, hydroxythiadiazole, sulfonamide, sulfonylcarboxamide, phosphonates, phosphonamides, phosphinates, sulfonates, acyl sulfonamide, mercaptoazole, acyl cyanamides.

### PDE7 Ligands and Inhibitors

The term “PDE7ligand” means a compound that binds to the PDE7 enzyme. Such compounds may be organic or inorganic compounds analogs or stereoisomers thereof, or other chemical or biological compounds, natural or synthesized, for example, peptides, polypeptides, proteins, including antibodies and antibody ligand binding domains, hormones, nucleotides, nucleic acids such as DNA or RNA, and further includes a pharmaceutically acceptable salt of the compound or stereoisomer, a prodrug of the compound or stereoisomer, or a pharmaceutically acceptable salt of the prodrug. A PDE7 ligand may also be a PDE7 inhibitor.

The term “PDE7 inhibitor” as used herein means a compound that acts to block the enzymatic activity of the PDE7. PDEs are enzymes that convert cyclic nucleotides, like cAMP, to the monoester forms. Several purines and particularly their methylated derivatives (theophylline, theobromine, caffeine) are potent cAMP phosphodiesterase inhibitors. Examples of suitable inhibitors include, organic compounds such as natural purines, or analogs thereof, or other compounds, organic or inorganic molecules, peptides, proteins, including antibodies and ligand binding domains of antibodies, nucleic acids such as DNA or RNA. Suitable examples of inhibitors of PDE7 may be for example organic compounds, or peptides or proteins, antibodies and fragments thereof peptidomimetic organic compounds that bind, for example, to the catalytic or regulatory domain of PDE7 and inhibit the activity triggered by the natural ligand substrate cAMP or the product AMP. The term inhibitor includes peptides and soluble peptides, including but not limited to members of random peptide libraries; (see, e.g., Lam et al., 1991, Nature 354:82-84; Houghten et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules. Suitable inhibitors may also be derived from diversity libraries, such as random or combinatorial peptide or nonpeptide, any libraries are known in the art that can be used, e.g., chemically synthesized libraries, recombinant (e.g., phage display libraries), and in vitro translation-based libraries. Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohimeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott & Smith, 1990, Science 249:386-390; Devlin et al., 1990, Science, 249:404-406; Christian, et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al/, 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO94/18318.

A compound which is PDE7 inhibitor may bind, and have effects, at the same site on PDE7 at which cAMP normally binds, although it may act at sites on PDE7remote to the cAMP binding site. Inhibitors of PDE7 may act to block the PDE7 activation by any suitable means such as for example, by binding to PDE7 or to cAMP or AMP or any other substrate or product ligand, and thereby inhibit the binding of cAMP or substrate ligand with PDE7. Such inhibitors may act in the place of cAMP at the PDE7, or may interact with, combine with or otherwise modify cAMP, thereby affecting how it acts at the PDE7. Alternatively the inhibitor can act to block PDE7 activity by affecting PDE7 gene expression, such inhibitors include, for example, molecules, proteins or small organic molecules or DNA or RNA, siRNA, that affect transcription or interfere with splicing events so that expression of the full length or the truncated form of PDE7 can be effected. Thus such PDE7 inhibitors can also include antisense RNA and sRNA products (silence interfering RNA).

The term “selective” means that a ligand or inhibitor binds with greater affinity to a particular enzyme when compared with the binding affinity of the ligand or inhibitor to another enzyme. Preferably, the binding affinity of the inhibitor for the first enzyme is about 50% or greater than the binding affinity for the second enzyme. More preferably, the binding affinity of the inhibitor to the first enzyme is about 75% or greater than the binding affinity to the second enzyme. Most preferably, the binding affinity of the inhibitor to the first enzyme is about 90% or greater than the binding affinity to the second enzyme. In a preferred embodiment of the invention, the inhibitor exhibits a greater binding affinity for the PDE7. Particularly preferred inhibitors are those that bind with greater affinity to the PDE7 enzyme when compared with binding to another PDE enzymes such as PDE 1, 3, 4, 5. It is contemplated that preferred inhibitors bind PDE7 with micromolar or greater affinity. More preferred inhibitors bind PDE7 with nanomolar or greater affinity. Preferred PDE7 inhibitors of the present invention include compounds or ligands that are selective inhibitors of PDE7. Selectivity can be determined based on comparative kinetic inhibition assays of inhibitors against different PDEs [Pitt, W J, et al Biorg. Med. Chem. Left, 14, 2004 2955-2958].

PDE7 ligands can be identified, for example, by screening a compound library. Methods of identifying inhibitors of enzymes are well known to those skilled in the art [Pitt, W J, et al Biorg. Med. Chem. Lett, 14, 2004 2955-2958, particularly reference 13 page 2958]. Specific procedures that can be used to identify PDE7 ligands are presented below.

According to the invention a PDE7 inhibitor can be used to treat neuropathic pain and the symptoms of neuropathic pain including hyperlagesia, allodynia and ongoing pain.

Physiological pain is an important protective mechanism designed to warn of danger from potentially injurious stimuli from the external environment. Neuropathic pain in particular arises from neurons that have themselves been damaged and has important elements which are mediated via activity in sensory nerves which do not normally convey pain, the Aβ neurones.

Neuropathic pain is defined as pain initiated or caused by a primary lesion or dysfunction in the nervous system (IASP definition). Nerve damage can be caused by trauma and disease and thus the term ‘neuropathic pain’ encompasses many disorders with diverse aetiologies. These include but are not limited to, Diabetic neuropathy, Post herpetic neuralgia, Back pain, Cancer neuropathy, HIV neuropathy, Phantom limb pain, Carpal Tunnel Syndrome, chronic alcoholism, hypothyroidism, trigeminal neuralgia, uremia, or vitamin deficiencies. Neuropathic pain is pathological as it has no protective role. It is often present well after the original cause has dissipated, commonly lasting for years, significantly decreasing a patients quality of life (Woolf and Mannion 1999 Lancet 353: 1959-1964). The symptoms of neuropathic pain are difficult to treat, as they are often heterogeneous even between patients with the same disease (Woolf & Decosterd 1999 Pain Supp. 6: S141-S147; Woolf and Mannion 1999 Lancet 353: 1959-1964). They include spontaneous pain, which can be continuous, or paroxysmal and abnormal evoked pain, such as hyperalgesia (increased sensitivity to a noxious stimulus) and allodynia (sensitivity to a normally innocuous stimulus).

The term “therapeutically effective amount” means an amount of a compound or combination of compounds that treats a disease; ameliorates, attenuates, or eliminates one or more symptoms of a particular disease; or prevents or delays the onset of one of more symptoms of the neuropathic pain.

The term “patient” means animals, such as dogs, cats, cows, horses, sheep, geese, and humans. Particularly preferred patients are mammals, including humans of both sexes.

The term “pharmaceutically acceptable” means that the substance or composition must be compatible with the other ingredients of a formulation, and not deleterious to the patient.

The terms “treating”, “treat” or “treatment” include preventative or prophylactic, and palliative treatment.

### Primary Binding Assays

In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by a modification of the method of Thompson W J and Appleman M M; Biochemistry 10(2), 311-316, 1971, as described by Ballard S A et al.; J. Urology 159(6), 2164-2171, 1998. In particular, cGMP-specific PDE5 and cGMP-inhibited cAMP PDE3 can be obtained from human corpus cavernosum tissue, human platelets or rabbit platelets; cGMP-stimulated PDE2 was obtained from human corpus cavernosum; calcium/calmodulin (Ca/CAM)-dependent PDE1 from human cardiac ventricle; cAMP-specific PDE4 from human skeletal muscle; and photoreceptor PDE6 from bovine retina. Phosphodiesterases 7-11 can be generated from full length human recombinant clones transfected into SF9 cells. Assays can be performed either using a modification of the “batch” method of Thompson, W J et al.; Biochemistry 18(23), 5228-5237, 1979, essentially as described by Ballard S A et al.; J. Urology 159(6), 2164-2171, 1998 or using a scintillation proximity assay for the direct detection of [³H]-labelled AMP/GMP using a modification of the protocol described by Amersham pic under product code TRKQ7090/7100. In summary, for the scintillation proximity assay the effect of PDE inhibitors was investigated by assaying a fixed amount of enzyme in the presence of varying inhibitor concentrations and low substrate, (cGMP or cAMP in a 3:1 ratio unlabelled to [³H]-labeled at a concentration of ˜⅓ Kₘ or less) such that IC₅₀≈K_{I}. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension.

PDE7 ligands and inhibitors can be identified, for example by screening a compound library and by employing a variety of screening techniques against PDE7. Methods of identifying ligands and inhibitors of the enzyme are known and examples of these are presented below:

The identification of test compounds as ligands of PDE7 and the affinity with which a test compound binds to the PDE7 may be determined through use of labelled ligand binding assays, for example standard radioligand binding assays, although other modes of labelling are available, wherein the test compound is labelled to detect binding, for example by radiolabelling, and incubated with a preparation of the target PDE7 enzyme. Such an enzyme preparation may be obtained from cells transfected with and expressing a recombinant PDE7 enzyme or chosen from a cell lysate of a cell line known to naturally express PDE7.

In a direct binding assay, PDE7 is contacted with a test compound under conditions that allow binding of the test compound to the PDE7. The binding may take place in solution or on a solid surface. Preferably, the test compound is previously labelled for detection. Any detectable group may be used for labelling, such as but not limited to, a luminescent, fluorescent, or radioactive isotope or group containing same, or a nonisotopic label, such as an enzyme or dye. After a period of incubation sufficient for binding to take place, the reaction is exposed to conditions and manipulations that remove excess or non-specifically bound test compound. Typically, this involves washing with an appropriate buffer. Finally, the presence a PDE7-test compound complex is detected. Alternatively binding interactions can be detected by measuring changes in changes in fluorescence on ligand displacement from the enzyme, change in protein fluorescence or molecular tumbling rate or molecular sedimentation in solution of the enzyme in the presence of test compound.

In a preferred embodiment of the direct binding assay, to facilitate complex formation and detection, the binding assay is carried out with one or more components immobilized on a solid surface. In various embodiments, the solid support could be, but is not restricted to, polycarbonate, polystyrene, polypropylene, polyethylene, glass, nitrocellulose, dextran, nylon, polyacrylamide and agarose. The support configuration can include beads, membranes, microparticles, the interior surface of a reaction vessel such as a microtitre plate, test tube or other reaction vessel. The immobilization of PDE7, or other component, can be achieved through covalent or non-covalent attachments. In one embodiment, the attachment may be indirect, i.e. through an attached antibody. In another embodiment, PDE7 is tagged with an epitope, such as glutatione S-transferase (GST) so that the attachment to the solid surface can be mediated by a commercially available antibody such as anti-GST (Santa Cruz Biotechnology). For example, such an affinity binding assay may be performed using a PDE7 which is immobilized to a solid support. Typically, the non-immobilized component of the binding reaction, in this case the test compound, is labelled to enable detection. A variety of labelling methods are available and may be used, such as detection of luminescent, chromophoric, fluorescent, or radioactive isotopes or groups, or detection of nonisotopic labels, such as enzymes or dyes. In one preferred embodiment, the test compound is labelled with a fluorophore such as fluorescein isothiocyanate (FITC, available from Sigma Chemicals, St. Louis). The labelled test compound, is then allowed to contact with the solid support with the immobilised PDE7, under conditions that allow specific binding to occur. After the binding reaction has taken place, unbound and non-specifically bound test compounds are separated by means of washing the surface. Attachment of the binding partner to the solid phase can be accomplished in various ways known to those skilled in the art, including but not limited to chemical cross-linking, non-specific adhesion to a plastic surface, interaction with an antibody attached to the solid phase, interaction between a ligand attached to the binding partner (such as biotin) and a ligand-binding protein (such as avidin or streptavidin) attached to the solid phase, and the like. Finally, the label remaining on the solid surface may be detected by any detection method known in the art. For example, if the test compound is labelled with a fluorophore, a fluorimeter may be used to detect complexes.

Alternatively, the binding reaction may be carried out in solution. In this assay, the labelled component is allowed to interact with its binding partner(s) in solution. If the size differences between the labelled component and its binding partner(s) permit such a separation, the separation can be achieved by passing the products of the binding reaction through an ultrafilter whose pores allow passage of unbound labelled component but not of its binding partner(s) or of labelled component bound to its partner(s) to determine levels of bound vs free ligand. Separation can also be achieved using any reagent capable of capturing a binding partner of the labelled component from solution, such as an antibody against the binding partner, a ligand-binding protein which can interact with a ligand previously attached to the binding partner, and so on.

Effects of a test compound on the catalytic activity of a PDE7 can be most easily determined by standard competitive binding experiments between PDE inhibitors and cAMP on enzyme activity for which known amounts of cAMP substrate and fixed amounts of enzyme are incubated together with various amounts of inhibitor substance for fixed periods of time, after which the reaction is stopped and the residual amount of unhydrolysed cAMP is measured. This may be done for any test sample by use of a scintillation proximity based assay (SPA) designed to measure the competition between cAMP in the test sample and a known amount of radiolabelled cAMP for binding to a cAMP-specific antibody attached to scintillant beads (Hancock, A. A., Vodenlich, A. D., Maldonado, C., Janis, R. (1995) a2-adrenergic agonist-induced inhibition of cyclic AMP formation in transfected cell lines using a microtiter-based Scintillation Proximity Assay. J. of Receptor and Signal Transduction research 15:557-579). The assay is read in a scintillation counter where the counts per sample are inversely related to the amount of cAMP present in the test sample. SPA kits for measurement of cAMP are available from Amersham Pharmacia Biotech (Amersham, UK).

Identification of inhibitor activity can be judged using a standard SPA (scintillation proximity assay) assay with a PDE7 enzyme. The PDE7 enzyme can be for example recombinant mouse, human or yeast or can be derived from a whole cell lysate of Hut78 Tcell line as a surrogate for the use of a recombinant PDE7A according to the method of Pitts, W J., et al Biorg. Med. Chem. Lett 14 2004 2955-2958. IC50 values of <1 micromolar in the presence of inhibitor are indicative of good inhibition.

In a preferred embodiment, a binding assay can be performed as follows:

Phosphodiesterase activity of PDE7 can be measured using the phosphodiesterase Scintillation Proximity Assay (SPA) (Amersham) according to the manufacturer's protocol, for convenience the assays can be done in triplicate in 96 well format. Reaction times and enzyme dilution are optimised so that the lowest substrate concentration gives no more than 30% conversion of substrate to product to ensure linearity. The reactions can contain for example 25 μl of the appropriately diluted enzyme, 25 μl buffer (20 mM Tris with 5 mM MgCL2.6H2O, pH 7.4 plus 2 mg/ml BSA) and initiated by the addition of 50 μl of either cAMP or cGMP to give a total reaction volume of 100 μl. [³H]-cAMP (Amersham Cat. No. TRK304 B70, 24 Ci/mmol) or [³H]-cGMP (Amersham Cat. No. TRK392 B37, 10.7 Ci/mmol) is mixed with the corresponding cold cyclic nucleotide to give a final concentration range of 1 μM-0.002 μM. This is achieved by performing doubling dilutions across a 96 well plate. Following a 40 min incubation at 30° C., the plates are immediately centrifuged at 2000 rpm for 5 min and then counted on TopCount. Background levels for each cAMP concentration were determined using a Scintillation Counter. Average counts of triplicate results for each assay are determined and the corresponding background subtracted. Counts per min for each assay are converted into pmol of cAMP hydrolysed per min per ml of enzyme and plotted against cAMP concentration (μM). For inhibitor profiling a concentration range of 0.5-300 μM in 1% dimethyl sulphoxide for each inhibitor is used and cAMP concentration is kept constant at ⅓ Kₘ. The assay blank contains all reagents minus the enzyme. Values for Kₘ and IC₅₀ were determined using the computer package GraFit4.

According to an alternative preferred embodiment, a binding assay can be performed as follows:

Inhibition of PDE activity can be determined using Hut78 cell lysate (Hut78 is a Tcell line which expresses PDE7) and an SPA specific for cAMP (Amersham Pharmacia Biotech, Buckinghamshire, UK) according to the manufacturers instructions with minor modifications. Enzyme assays are performed at room temperature in the presence of 50 mM Tris-HCl, pH7.5, containing 8.3 mM MgCl₂, 1.7 mM EGTA, and 0.5 mg/mL BSA. Each assay is performed in a 100 μL reaction volume in 96 well microtitre plates containing the above buffer, 0.3 μL of Hut78 cell lysate treated with 2 μM Zardaverine to inhibit PDE3 and PDE4, 0.05 μCi of [5′,8-³H] Adenosine 3′,5-cyclic phosphate as an ammonium salt for 20 min. Inhibitors are included at a concentration range of 0.5-300 μM for each inhibitor is used and cAMP concentration is kept constant, the assay blank contains all reagents minus the enzyme. The reaction was terminated by the addition of 50 μL PDE SPA beads (1 mg) water with 10 mM cold cAMP (Sigma, St. Louis Mo.). The reaction mix was allowed to settle for 20 min before counting in a Top Count-NXT scintillation counter (Packard BioScience, Meriden, Conn.). For selectivity studies, the assay is essentially unchanged except that ³H-cyclic GMP is used as the substrate for PDE1, PDE5, and PDE6. The following PDEs/activators and enzyme sources are used: PDE1, bovine (Sigma St. Louis), calmodulin; PDE2, rat kidney, cGMP; PDE3, human platelet; PDE4, rat kidney; PDE5, human platelet, and PDE6, bovine retina.

### Selectivity of Inhibitors

The compounds of the invention are PDE7 inhibitors and are preferably potent PDE7 inhibitors. These compounds have low IC₅₀ values for PDE7, typically at less than 100 nM, preferably less than 10 nM, more preferably less than 1 nM.

The compounds of the invention are PDE7 inhibitors and are preferably selective PDE7 inhibitors. The selectivity of PDE7 inhibitor is preferably at least 10 fold selective for PDE7 over other PDEs, preferably it should be at least 100 fold selective and further preferably at least 1000 fold selective. Selectivity in general represents the relative potency of a compound between two enzyme subtypes for the appropriate ligand or inhibitor for the enzyme of interest.

A PDE7 ligand or inhibitor, can be tested for selectivity for the PDE7 in comparison with another PDE such as for example PDE4. In the assay, the capacity of each test compound to compete with binding of labelled-cAMP is measured at both the PDE7 and PDE4 enzymes, and an IC₅₀ value (in μ) is determined. Any of the above mentioned binding assay procedures can be used. For example in an inhibition assay, test compounds are assayed for their ability to disrupt the binding and hydrolysis of cAMP by PDE7. Labelled cAMP may be mixed with PDE7 or a fragment or derivative thereof, and placed under conditions in which the interaction between them would normally occur, either with or without the addition of the test compound. The amount of labelled cAMP that binds and is hydrolysed by PDE7 or PDE4 may be compared to the amount bound and hydrolysed in the presence or absence of test compound, thus the level of inhibition of the process can be determined for any test compound addition at either PDE and compared.

The potency of a PDE7 inhibitor (based on IC50 potency which can be defined as the concentration of inhibitor that gives a halving of the value of the functional activity of a enzyme in a functional assay as described below) is preferably at least 100 nM IC50 at the human enzyme (recombinant and/or native), more preferably less than 10 nM and further preferably less than 1 nM. For instance in a functional cell based assay, IC50 is the molar concentration of an inhibitor that inhibits by 50% the maximal activity of the human PDE7 for example in response to cAMP. In a binding assay, IC50 is the molar concentration of an inhibitor that displaces 50% of the specific binding of labelled cAMP or other appropriate ligand or the molar concentration at which the test compound occupies half of the available PDE7 binding sites.

### Functional Assays

Functional assay methods are known for identifying a compounds that are inhibitors of PDE7. The methods generally include the steps comprising: a) contacting a PDE7-expressing cell with a test compound optionally in the presence of cAMP or another PDE7 substrate ligand; and b) measuring the resultant level of a PDE7 activity, or the level of expression of PDE7 in the cell, such that if said level of measured activity or expression differs from that measured in the absence of the test compound, then a compound that modulates a PDE7-cAMP-mediated process is identified. The PDE7 activity measured can be the ability to interact with cAMP or by a change in cAMP/AMP levels in the cell or the response of the cell to cAMP for example by alterations in gene transcription or protein activity. Example protocols for functional assays are provided below.

The key advantage of functional cell based assays is that they facilitate early and direct pharmacological characterization of compounds by high-throughput quantification and allow identification of compounds that act both at the binding site of the PDE or on a modulatory binding site on a PDE that is topographically distinct from the binding site. The most common systems of functional cell based assays are based on cyclic AMP detection and are reviewed in Williams, C., Nature Reviews Drug Discovery 3 2004 125-135. Cell-based assays in HTS provides the advantage of having the ability to identify inhibitor compounds and to obtain additional information about the mode of action of the compound.

HTS-compatible accumulation assays for cAMP measurement follow a general principle, with changes in intracellular cAMP being detected by the competition between cellular cAMP and a labelled form of cAMP for binding to an anti-cAMP sequestering antibody or directly to the PDE. Protocols for these assays differ markedly and include: radiometric assays, fluorescence polarization cAMP assays, time-resolved fluorescence assays, assays which detect alterations in gene transcription or protein activity for example via initiation of phosphorylation events that regulate target enzymes and transcription factors, enzymatic assays, assays to determine binding to protein kinases within the cell. Homogeneous radiometric assays, such as scintillation proximity assays (SPA, Amersham Biosciences) and Flashplate technology (NEN/Perkin Elmer) enable the direct detection of [125I]-labelled cAMP once it is inclose proximity to a solid scintillant surface [Amersham Life Science. High throughput screening for cAMP formation by scintillation proximity radioimmunoassay. Proximity News Issue No. 23. (1996).&. NEN Life Science Products. A novel adenylyl cyclaseactivation assay on FlashPlate (Flasplate File #1, Application Note). (NEN Life Science Products Inc., Boston, Mass., 1998). 18. Kariv, I. I. et al. High throughput quantitation of cAMP production mediated by activation of seven transmembrane domain receptors. J. Biomol. Screen. 4, 27-32 (1999)].

Fluorescence polarization cAMP assays (available in kit form from companies such as Perkin Elmer and Amersham Biosciences) monitor the light emitted from a fluorescently tagged cAMP molecule following excitation with a polarized light source, the assays is based on a decrease in the extent of molecular rotation of a fluorescently labelled cAMP that occurs following binding to the larger anti-cAMP antibody. Alternatively, dyes such as Bodipy-TMR, MR121, Alexa, Cy3 and Cy5 have been used in FP binding assays.

The HTRF (homogeneous time-resolved fluorescence) technology uses anti-cAMP antibodies labelled with europium cryptate and cAMP that is labelled with a modified allophyocyanin (see the CIS Bio International HTRF web site). In the absence of cellular cAMP, these two fluorescent molecules are in close proximity, FRET occurs and long lifetime fluorescence is emitted at two different wavelengths. When the two molecules are separated by competition with cellular cAMP, no FRET occurs and only emission from the europium is detected. This technique has been successfully applied to high-throughput screening with whole cells in miniaturized formats. [Claret E, Roux P, Ouled-Diaf J, Préaudat C, Drexler C, Grépin C, Seguin P. Phosphodiesterase assays with HTRF® 10th SBS annual conference. September 2004, Orlando, US. Cisbio]

Additionally changes in the intracellular levels of cAMP produce alterations in gene transcription or protein activity and result in the observed functional response of the cell; these events can be measured via transcription factors such as NFAT (nuclear factor activated in T-cells) or CREB (cAMP response element binding protein) and reporter genes under the control of appropriate upstream elements [Hill, S. J. et al. Reporter-gene systems for the study of G-protein-coupled receptors. Curr. Opin. Pharmacol. 1, 526-532 (2001). 29. Wood, K. V. Marker proteins for gene expression. Curr. Opin. Biotechnol. 6, 50-58 (1995). 30. Southward, C. M. & Surett, M. G. The dynamic microbe:green fluorescen.

Reporter-gene assays for cAMP detection Reporter-gene assays follow a general principle, where by receptor-mediated changes in intracellular cAMP con-centrations are detected via changes in the expression level of a particular gene (the reporter), the transcription of which is regulated by the transcription factor cAMP response-element binding protein (CREB) binding to upstream cAMP response elements (CREs). Various reporter genes have been used in in vitro and in vivo studies, including β-galactosidase, green fluorescent protein (GFP), luciferase and β-lactamase 28-31. The reporter-gene method is compatible with screening for activity in live cells or enabling transfected cell populations. Cell lines commonly used in reporter-gene assays are for example Chinese hamster ovary cells (CHO) and human embryonic kidney cells.

Recently, three innovative technologies have emerged that also aim to provide non-radiometric high-sensitivity assays of cAMP accumulation. The first of these—ALPHAScreen (amplified luminescent proximity homogeneous assay; PackardBioscience/Perkin Elmer)—is a homogeneous assay format using chemiluminscent readout. The second system—an enzyme complementation technology from DiscoveRx (Fremont, Calif.)—uses a cAMP molecule tagged with an inactive β-galactosidase component and uses fluorescent or luminescent readout. The third system uses electrochemiluminescence detection and is a technology available from Meso ScaleDiscovery (Gaithersburg, Md.). In this case, the cAMP, is tagged with a ruthenium derivative, which results in the production of light from the labelled cAMP (see Meso Scale Discovery web site).

### In Vivo Procedures

The analgesic effect of PDE7 inhibitors may be determined in vivo using animal models of selected pain conditions. Several models of pain conditions are known and specific procedures that can be used to determine the analgesic effect of PDE7 inhibitors are presented below.

An alternative pain model is the streptozocin induced diabetic model of neuropathic pain in rats. This procedure involves administration of streptozocin (50 mg/kg, i.p.) in a single dose to animals such as Charles River Sprague dawley rats (225-250 g) to induce diabetes. Animals are evaluated 2 weeks following administration using static and dynamic allodynia tests and if neuropathic pain is confirmed they are used to further evaluate compounds for their effect on neuropathic pain (S. R. Chen and H. L. Pan. J. Neurophysiol. (2002), 87, 2726-2733).

The chronic constrictive injury (CCI) model of neuropathic pain in rats involves the tying of loose ligatures around the sciatic nerve Charles River male Sprague dawley rats (175-200 g) are placed in, an anaesthetic chamber and anaesthetised with a 2% isofluorane O₂ mixture. The right hind thigh is shaved and swabbed with 1% iodine. Animals are then transferred to a homeothermic blanket for the duration of the procedure and anaesthesia maintained during surgery via a nose cone. The skin is cut along the line of the thigh bone. The common sciatic nerve is exposed at the middle of the thigh by blunt dissection through biceps femoris. Proximal to the sciatic trifurcation, about 7 mm of nerve is freed by inserting forceps under the nerve and the nerve gently lifted out of the thigh. The forceps are gently opened and closed several times to aid clearance of the fascia from the nerve. Suture is pulled under the nerve using forceps and tied in a simple knot until slight resistance is felt and then double knotted. The procedure is repeated until 4 ligatures (4-0 silk) are tied loosely around the nerve with approx 1 mm spacing. The incision is closed in layers. Fourteen days following surgery, animals are assessed for static allodynia, dynamic allodynia or weight bearing deficit (G. J. Bennett and Y. K. Xie, Pain (1988) 33, 87-107).
Alternative animal models of neuropathic pain conditions include the Seltzer model, partial tight ligation of the sciatic nerve (Seltzer, Z. (1995). Sem. Neurosci, 8: pp. 34-39) or Chung's model, tight ligation of one of the two spinal nerves of the sciatic nerve (Kim S H, Chung J M. Pain (1992); 50: pp. 355-63) or of the Chronic Constrictive Injury model (CCI) (Bennett G J, Xie Y-K. Pain (1988); 33: pp. 87-107).

Alternative animal models of neuropathic pain conditions may involve selection of an animal that naturally possesses a painful disease condition providing neuropathic pain and its symptoms such as HIV or Herpes or cancer or diabetes. Alternatively the animal may be arranged to experience a pain condition by modification of the animal to possess a pain inducing disease condition such as arthritis or HIV or Herpes or cancer or diabetes. Animals may be modified to possess a pain condition due to a disease in a variety of ways for example by administration of Streptozocin to induce a diabetic neuropathy (Courteix, C., Eschalier, A., Lavarenne, J., Pain, 53 (1993) pp. 81-88.) or by administration of viral proteins to cause HIV related neuropathic pain (Herzberg U. Sagen J., Journal of Neuroimmunology. (2001 May 1), 116(1): pp. 29-39) or administration of varicella zoster virus to cause Herpes and post herpatic neuralgia (Fleetwood-Walker S M. Quinn J P. Wallace C. Blackburn-Munro G. Kelly B G. Fiskerstrand C E. Nash A A. Dalziel R G., Journal of General Virology. 80 (Pt 9):2433-6, 1999 Sep.) or administration of a carcinogen or of cancer cells to an animal to cause cancer (Shimoyama M. Tanaka K. Hasue F. Shimoyama N, Pain. 99(1-2): pp. 167-74, 2002 September).

Dynamic allodynia can be assessed by lightly stroking the plantar surface of the hind paw of the animal with a cotton bud. Care is taken to perform this procedure in fully habituated rats that are not active, to avoid recording general motor activity. At least two measurements are taken at each time point, the mean of which represents the paw withdrawal latency (PWL). If no reaction is exhibited within 15 s the procedure is terminated and animals are assigned this withdrawal time. Thus, 15 s effectively represents no withdrawal. A withdrawal response is often accompanied with repeated flinching or licking of the paw. Dynamic allodynia is considered to be present if animals responded to the cotton stimulus within 8 s of commencing stroking.

Following baseline evaluation, animals can be administered compounds for analgesic assessment by one of the following routes, oral administration, subcutaneous., intra-peritoneal., intravenous or intra-thecal. The PWL is re-evaluated at some or all of the following time points, 30 min, 1 h, 2 h, 3 h, 4 h, 5 h, 6 h, 7 h, 24 h. Animals are assigned randomly to each compound group according to their baseline values. The mean and standard error mean are calculated for each compound group at each time point. Measures of dynamic allodynia are compared to their respective controls using a one way ANOVA followed by a Dunnett's t-test comparing vehicle to compound at each time point. The minimum number of animals per group is 6 (M. J. Field et al. Pain (1999), 83, 303-11).

Static allodynia can be evaluated by application of von Frey hairs (Stoelting, Wood Dale, Ill., USA) in ascending order of force (0.6, 1, 1.4, 2, 4, 6, 8, 10, 15 and 26 grams) to the plantar surface of hind paws. Animals are habituated to wire bottom test cages prior to the assessment of allodynia. Each von Frey hair is applied to the paw for a maximum of 6 seconds, or until a withdrawal response occurs. Once a withdrawal response to a von Frey hair is established, the paw is re-tested, starting with the filament below the one that produces a withdrawal, and subsequently with the remaining filaments in descending force sequence until no withdrawal occurs. The highest force of 26 g lifts the paw as well as eliciting a response, thus representing the cut off point. Each animal has both hind paws tested in this manner. The lowest amount of force required to elicit a response is recorded as paw withdrawal-threshold (PWT) in grams. Static allodynia is defined as present if animals responded to a stimulus of, or less than, 4 g, which is innocuous in normal rats.

Following baseline evaluation, animals are administered compounds for analgesic assessment by one of the following routes, orally, subcutaneous, intra-peritoneal., intra-venous or intra-thecal. and the PWT re-evaluated at some or all of the following time points, 30 min, 1 h, 2 h, 3 h, 4 h, 5 h, 6 h, 7 h, 24 h. Static allodynia measurements are analysed using a Kruskall-Wallis test for non-parametric results, followed by Mann-Whitney's U test vs vehicle group. The minimum number of animals per group is 6 (M. J. Field et al. Pain (1999), 83, 303-11). Thermal hyperalgesia is assessed using the rat plantar test (Ugo Basile, Italy) following a modified method of Hargreaves et al., (1988) Pain 32:77-88. Rats are habituated to the apparatus that consists of three individual perspex boxes on an elevated glass table. A mobile radiant heat source is located under the table and focused onto the hind paw and paw withdrawal latencies (PWL) are recorded. There is an automatic cut off point of 22.5 to prevent tissue damage. PWL are taken 2-3 times for both hind paws of each animal, the mean of which represented baselines for right and left hind paws. The apparatus is calibrated to give a PWL of approximately 10 s. PWL are reassessed 2 h following administration of carrageenan. Following administration of compounds for analgesic assessment PWL's are reassessed hourly for up to 6 hours. PWL's of compound groups are compared to their respective controls using a one way ANOVA followed by a Dunnett's t-test. The minimum number of animals per group will be 6.

Weight bearing deficit can be measured according to the method of: Bove S E, et. al. Weight bearing as a measure of disease progression and efficacy of anti-inflammatory compounds in a model of monosodium iodoacetate-induced osteoarthritis. Osteoarthritis Cartilage. 2003 November; 11(11):821-30. Open field test can be carried out according to the method of Prut L and Beizung, C. The open field as a paradigm to measure the effects of compounds on anxiety-like behaviors: a review. Eur J Pharmacol. 2003; 463:3-33. The locomotor test can be carried out according to the method of Salmi P and Ahlenius S-Sedative effects of the dopamine D1 enzyme agonist A 68930 on rat open-field behavior. Neuroreport. 2000 Apr. 27; 11(6):1269-72.

### Combinations

A PDE7 inhibitor may be usefully combined with another pharmacologically active compound, or with two or more other pharmacologically active compounds, in the treatment of neuropathic pain. For example, a PDE7 inhibitor, particularly a compound of general formulae, or a pharmaceutically acceptable salt or solvate thereof, as defined above, may be administered simultaneously, sequentially or separately in combination with one or more agents selected from:
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl)quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT_{1B/1D} agonist such as eletriptan, sumatriptan, naratriptan, zolmitriptan or rizatriptan;
a 5-HT_{2A} receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′, 1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine;
a 5-HT3 antagonist, such as ondansetron;
   and the pharmaceutically acceptable salts and solvates thereof.

A PDE7 inhibitor is administered to a patient in a therapeutically effective amount. A PDE7 inhibitor can be administered alone or as part of a pharmaceutically acceptable composition, in the treatment of neuropathic pain.

### Drug Substance

A PDE7 inhibitor of the present invention, for example a compound of the general formulae, can be administered in the form of a pharmaceutically acceptable salt, for instance an acid addition or a base salt.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, Weinheim, Germany, 2002).

Pharmaceutically acceptable salts may be prepared by one or more of three methods:
(i) by reacting a compound with the desired acid or base;
(ii) by removing an acid- or base-labile protecting group from a suitable precursor of a compound or by ring-opening a suitable cyclic precursor, for example, a lactone or lactam, using the desired acid or base; or
(iii) by converting one salt of a compound to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288, by Haleblian (August 1975).

Hereinafter all references to a PDE7 inhibitor of the present invention, for example a compound of the general formulae, include references to salts, solvates and complexes thereof and to solvates and complexes of salts thereof.

A PDE7 inhibitor of the present invention, for example a compound of the general formulae, may be administered in the form of a prodrug. A prodrug is a compound which may have little or no pharmacological activity itself but which can, when administered into or onto the body, be converted into a compound having the desired activity, for example, by hydrolytic cleavage. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs can, for example, be produced by replacing appropriate functionalities present in a compound with certain moieties known to those skilled in the art as ‘pro-moieties’ as described, for example, in Design of Prodrugs by H. Bundgaard (Elsevier, 1985).

Some examples of prodrugs include
(i) where a compound contains a carboxylic acid functionality (—COOH), an ester thereof, for example, a compound wherein the hydrogen of the carboxylic acid functionality of the compound of the general formulae is replaced by (C₁-C₈)alkyl;
(ii) where a compound contains an alcohol functionality (—OH), an ether thereof, for example, a compound wherein the hydrogen of the alcohol functionality of the compound is replaced by (C₁-C₆)alkanoyloxymethyl; and
(iii) where a compound contains a primary or secondary amino functionality (—NH₂ or —NHR where R≠H), an amide thereof, for example, a compound wherein, as the case may be, one or both hydrogens of the amino functionality of the compound is/are replaced by (C₁-C₁₀)alkanoyl.

Further examples of replacement groups in accordance with the foregoing examples and examples of other prodrug types may be found in the aforementioned references.

Moreover, certain compounds may themselves act as prodrugs of other compounds.

Also included within the scope of the invention are metabolites of a PDE7 inhibitor of the present invention, for example a compound of the general formulae, that is, compounds formed in vivo upon administration of the drug. Some examples of metabolites in accordance with the invention include
(i) where a compound contains a methyl group, an hydroxymethyl derivative thereof (—CH₃->—CH₂OH):
(ii) where a compound contains an alkoxy group, an hydroxy derivative thereof (—OR->—OH);
(iii) where a compound contains a tertiary amino group, a secondary amino derivative thereof (—NR¹R²->—NHR¹ or —NHR²);
(iv) where a compound contains a secondary amino group, a primary derivative thereof (—NHR¹->—NH₂);
(v) where a compound contains a phenyl moiety, a phenol derivative thereof (-Ph->-PhOH); and
(vi) where a compound contains an amide group, a carboxylic acid derivative thereof (—CONH₂->COOH).

A PDE7 inhibitor of the present invention, for example a compound of the general formulae, containing one or more asymmetric carbon atoms can exist as two or more stereoisomers. Where a compound contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of the general formulae containing, for example, an imino, keto, or oxime group, or so-called valence tautomerism in compounds which contain an aromatic moiety. It follows that a single compound may exhibit more than one type of isomerism.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of the general formulae contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art—see, for example, Stereochemistry of Organic Compounds by E. L. Eliel and S. H. Wilen (Wiley, New York, 1994).

A PDE7 inhibitor of the present invention, for example a compound of the general formulae, may exist in one or more isotopic forms wherein one or more atoms are replaced by atoms having the same atomic number, but an atomic mass or mass number different from the atomic mass or mass number which predominates in nature.

Examples of isotopes include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate enzyme occupancy.

Isotopically-labeled compounds can generally be prepared by conventional techniques.

Pharmaceutically acceptable solvates in accordance with the invention include those wherein the solvent of crystallization may be isotopically substituted, e.g. D₂O, d₆-acetone, d₆-DMSO.

### Drug Product

A PDE7 inhibitor of the present invention, for example a compound of the general formulae, intended for pharmaceutical use may be administered as a crystalline or amorphous product. It may be obtained, for example, as a solid plug, powder, or film by methods such as precipitation, crystallization, freeze drying, spray drying, or evaporative drying. Microwave or radio frequency drying may be used for this purpose.

It may be administered alone or in combination with one or more other compounds of the invention or in combination with one or more other drugs (or as any combination thereof). Generally, it will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of a PDE7 inhibitor of the present invention, for example a compound of the general formulae, and methods for its preparation will be readily apparent to those skilled in the art. Such compositions and methods for its preparation may be found, for example, in Remington's Pharmaceutical Sciences, 19th Edition (Mack Publishing Company, 1995).

### Oral Administration

A PDE7 inhibitor of the present invention, for example a compound of the general formulae, may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

A PDE7 inhibitor of the present invention, for example a compound of the general formulae, of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980). Consumable oral films for human or veterinary use are typically pliable water-soluble or water-swellable thin film dosage forms which may be rapidly dissolving or mucoadhesive and typically comprise a compound of the general formulae, a film-forming polymer, a binder, a solvent, a humectant, a plasticiser, a stabiliser or emulsifier, a viscosity-modifying agent and a solvent. Some components of the formulation may perform more than one function.

A PDE7 inhibitor of the present invention, for example a compound of the general formulae, may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, a PDE7 inhibitor of the present invention, for example a compound of the general formulae, may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

### Parenteral Administration

A PDE7 inhibitor of the present invention, for example a compound of the general formulae, may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of a PDE7 inhibitor of the present invention, for example a compound of the general formulae, used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus a PDE7 inhibitor of the present invention, for example a compound of the general formulae, may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

### Topical Administration

A PDE7 inhibitor of the present invention, for example a compound of the general formulae, may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

### Inhaled/Intranasal Administration

A PDE7 inhibitor of the present invention, for example a compound of the general formulae, can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of a PDE7 inhibitor of the present invention, for example a compound of the general formulae, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a PDE7 inhibitor of the present invention, for example a compound of the general formulae, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. The overall daily dose may be administered in a single dose or, more usually, as divided doses throughout the day.

### Rectal/Intravaginal Administration

A PDE7 inhibitor of the present invention, for example a compound of the general formulae, may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

Ocular/Aural Administration

A PDE7 inhibitor of the present invention, for example a compound of the general formulae, may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

### Other Technologies

A PDE7 inhibitor of the present invention, for example a compound of the general formulae, may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

### Kit-Of-Parts

Inasmuch as it may desirable to administer a combination of active compounds, for example, for the purpose of treating a particular disease or condition, it is within the scope of the present invention that two or more pharmaceutical compositions, at least one of which contains a PDE7 inhibitor of the present invention, for example a compound of the general formulae, may conveniently be combined in the form of a kit suitable for coadministration of the compositions.

Thus the kit of the invention comprises two or more separate pharmaceutical compositions, at least one of which contains a PDE7 inhibitor of the present invention, for example a compound of the general formulae, in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

### Dosage

For administration to human patients, the total daily dose of a PDE7 inhibitor of the present invention, for example a compound of the general formulae, is typically in the range 0.1 mg to 1 g depending, of course, on the mode of administration. The element of the pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsules, tablet, cachet, or lozenge itself, or it can be the appropriate number of any of these in packaged form. The quantity of active component in a unit dose preparation may be varied or adjusted from 0.1 mg to 1 g according to the particular application and the potency of the active components. In medical use the drug may be administered one to three times daily as, for example, capsules of 100 or 300 mg. In therapeutic use, the compounds utilized in the pharmaceutical method of this invention are administered at the initial dosage of about 0.01 mg to about 100 mg/kg daily. A daily dose range of about 0.01 mg to about 100 mg/kg is preferred. The total daily dose may be administered in single or divided doses and may, at the physician's discretion, fall outside of the typical range given herein.

These dosages are based on an average human subject having a weight of about 60 kg to 70 kg. The physician will readily be able to determine doses for subjects whose weight falls outside this range, such as infants and the elderly.

For the avoidance of doubt, references herein to “treatment” include references to curative, palliative and prophylactic treatment.

The following examples illustrate the embodiments and principles of the invention:

### EXAMPLES

### General Methods with Reference to the Compounds of Formula (IV)

All of the compounds of formula (IV) can be prepared by the procedures described in the General Methods described below or by the specific methods described in the Examples section and the Preparations section, or by routine modifications thereof. The present invention also encompasses any one or more of these processes for preparing the compounds of formula (IV), in addition to any novel intermediates used therein.

The following abbreviations are used:

DMF=dimethylformamide
DMSO=dimethyl sulphoxide
TEMPO=2,2,6,6-tetramethylpiperidine-N-oxide
THF=tetrahydrofuran

The compounds of formula (IV) may be prepared as shown in Scheme 1 below.

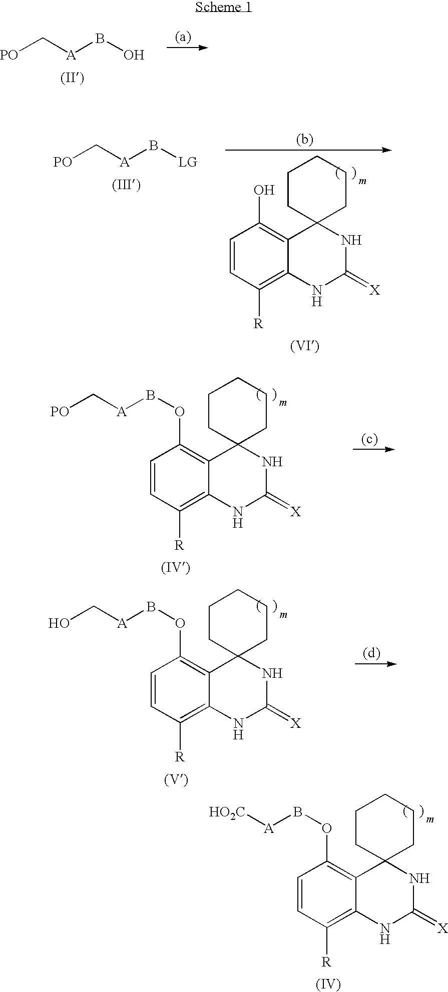

In Scheme 1, P represents a hydroxy-protecting group, suitable examples of which are described in “Protective Groups in Organic Synthesis” by T. W. Greene and P. Wuts, Wiley and Sons, 1991, and LG represents a suitable leaving group, such as halogen or sulphonate (eg methanesulphonate, p-toluenesulphonate or trifluoromethanesulphonate). Preferably P is benzyl and LG is p-toluenesulphonate.

Step (a): The compound of formula (III′) may be prepared from compound (II) and an appropriate agent capable of converting a hydroxy group into a leaving group, typically a sulphonylating reagent (eg methanesulphonyl chloride or p-toluenesulphonyl chloride) in the presence of a base (eg triethylamine or pyridine) in a suitable solvent (eg pyridine or dichloromethane) at 0° C. to room temperature for 15 minutes to 24 hours.

Preferred conditions are: 1 eq compound (II′) in dichloromethane, 1.2 eq p-toluenesulphonyl chloride, 2 eq pyridine at room temperature for 18 hours.

Step (b): The compound of formula (IV′) may be prepared from compound (III′) and the hydroxy compound of formula (VI′) in a suitable solvent (eg DMF, DMSO) in the presence of a suitable base (eg Cs₂CO₃, K₂CO₃), optionally in the presence of a crown ether (eg 18-crown-6) at 50-120° C. overnight.

Preferred conditions are: 1 eq compound (VI′), 1.1eq compound (III′), 1.2 eq Cs₂CO₃, in DMF at 80° C. for 24 hours.

Compounds of formula (VI′) are preferred embodiments of compounds of formulae (I) (II) and (III) generally described in WO 02/074754. Specific compounds of formula (VI′) wherein X is O, m is 1 and R is Cl may be prepared as described in Bioorg. Med. Chem. Lett., (2004), 14 (18), 4627-32.

Step (c): The compound of formula (IV′) may be deprotected by reaction with a deprotecting agent in a suitable solvent to yield the compound of formula (V′). Suitable reagents and methods are described in “Protective Groups in Organic Synthesis” (referred to above). When P is benzyl, examples of suitable reagents include boron trichloride or iron (III′) chloride.

Preferred conditions are: 1 eq compound (IV′) in dichloromethane, 4 eq BCl₃ at room temperature for 18 hours.

Step (d): The compound of formula (IV) may be prepared by oxidation of the compound of formula (V′) using an oxidising agent in a suitable solvent. Typical reagents and conditions include catalytic chromium trioxide and periodic acid (H₅IO₆) in a solvent such as acetonitrile at room temperature to 50° C. for 18 to 36 hours, or alternatively NaOCl plus NaClO₂ in the presence of catalytic TEMPO in a solvent such as acetonitrile at 0° C. to room temperature for 18 to 36 hours.

Preferred conditions are: 1 eq compound (V′), 2.5 eq periodic acid, 0.02 eq CrO₃, in 0.75% aqueous acetonitrile, 24 hours at 40° C.

The compounds of formula (IV) may alternatively be prepared by oxidation of compounds of formula (V′) in a two-step procedure via the aldehydes of formula (VII′) as shown in Scheme 2.

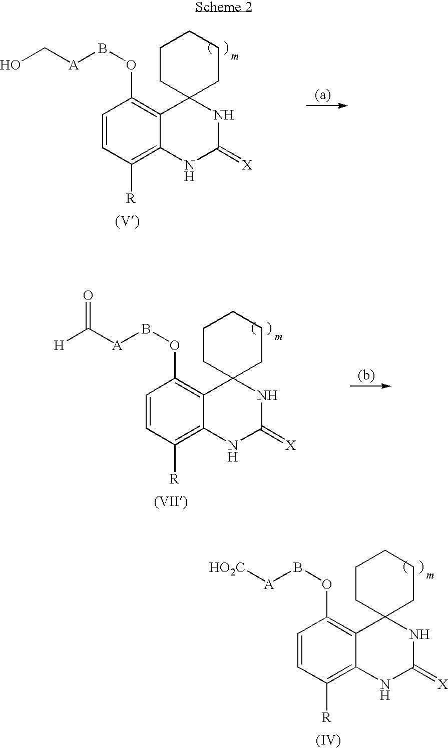

Step (a): Oxidation of the alcohol (V′) to the aldehyde (VII′) is typically carried out using NaOCl with catalytic TEMPO in a suitable solvent, eg acetonitrile, acetone at 0° C. to room temperature for 2-18 hours, or alternatively using sulphur trioxide-pyridine complex with DMSO in a solvent such as THF at 0° C. to room temperature for 2-18 hours.

Step (b): Further oxidation of the aldehyde (VII′) to the acid (IV) with is typically carried out using NaClO₂ in the presence of potassium phosphate in a solvent such as aqueous t-butanol at 0° C. to room temperature for 2-18 hours, or alternatively using trichloroisocyanuric acid with catalytic TEMPO in a suitable solvent, eg acetone or acetonitrile, at 0° C. to room temperature for 2-18 hours.

Compounds of formula (II′) are known in the literature. For example, compounds of formula (II′) wherein A is a cis-1,3-cyclobutylene group and B is a single bond may be prepared as described in J. Chem. Soc., Perkin Trans. 1, (1995), 18, 2281-7.

Alternatively compounds of formula (Ib), which are compounds of formula (IV) wherein A is a cis- or trans-1,3-cyclobutylene group and B is a single bond may be prepared from compound (VIII′) or compound (IX′) by standard methods, such as shown in Scheme 3.

Trans compounds (II′) and (X₁) may be obtained from cis compounds (II′) and (X₁) respectively by inversion using Mitsunobu chemistry analogous to that described in Synthesis, (1981), 1.

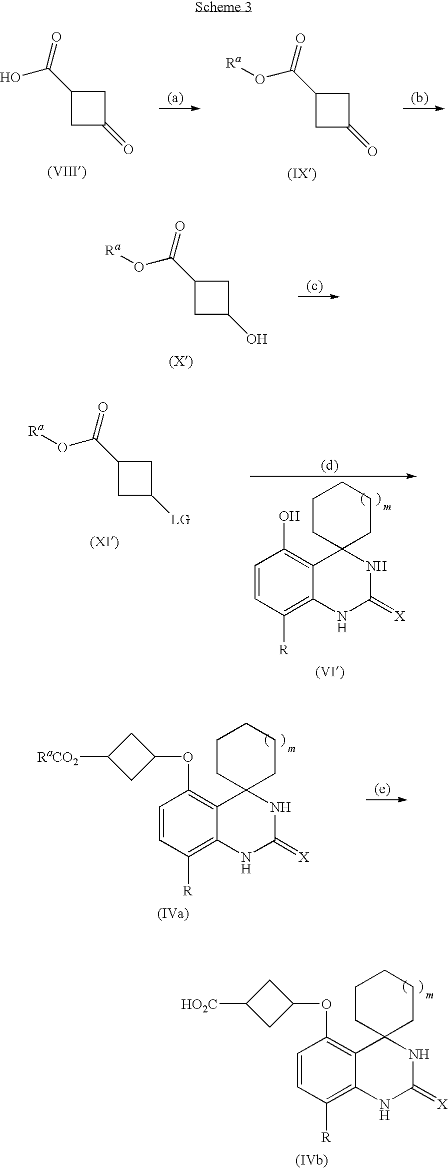

In Scheme 3, R^{a} is an ester residue, suitable examples of which are described in “Protective Groups in Organic Synthesis” (referred to above) (eg (C₁₋₆)alkyl, benzyl or (+) or (−)-menthyl), and LG is a leaving group such as halogen or sulphonate (eg methanesulphonate, p-toluenesulphonate or trifluoromethanesulphonate).

Step (a): The compound of formula (IX′) may be prepared by reaction of compound (VII′) with a suitable alcohol of formula R^{a}OH (eg methanol, t-butanol, (−) menthol) under a variety of conditions, suitable examples of which are described in “Protective Groups in Organic Synthesis” (referred to above).

Preferred conditions are: 1 eq compound (VII′), 1.1 eq. 1,1′-carbonyl diimidazole, in ethyl acetate at reflux for 1 hour followed by 1 eq R^{a}OH at room temperature for 4 hours.

Step (b): Reduction of compound (IX′) to the alcohol (X′) may be carried out using a suitable reducing agent, eg sodium borohydride or L-Selectride®, in a suitable solvent such as THF.

Preferred conditions are: 1 eq compound (IX′), 0.5 eq NaBH₄ in 20:1 THF:methanol at 0° C. for 20 minutes.

Step (c): The compound of formula (XI′) may be prepared from compound (X′) using reagents and conditions similar to those described in Scheme 1, step (a).

Preferred conditions are: 1 eq compound (X′), 1.05 eq p-toluenesulphonyl chloride in pyridine at 0° C. to room temperature.

Step (d): The compound of formula (Ia) may be prepared from compound (XI′) and the hydroxy compound of formula (VI′) using reagents and conditions similar to those described in Scheme 1, step (b).

Preferred conditions are: 1.2 eq compound (XI′), 1.0 eq compound (VI′), 1.5 eq Cs₂CO₃ in DMF at 80° C. for 18 hours.

Step (e): The compound of formula (Ia) may be hydrolysed to provide the compound of formula (Ib). This reaction may be achieved under a variety of conditions, suitable examples of which are described in “Protective Groups in Organic Synthesis” (referred to above).

Preferred conditions are: compound (Ia), 2 eq NaOH in 1:1 ethanol:water at 60° C. for 2 hours.

Compound (VIII′) is described in J. Org. Chem., (1981), 53, 3841-43 and compound (IX′) is described in J. Org. Chem., (1994), 59, 2132-34.

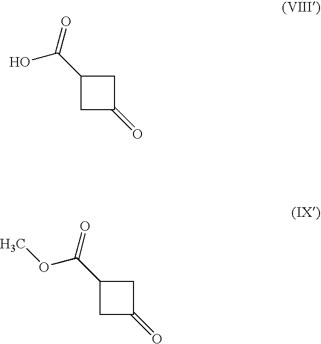

¹H Nuclear magnetic resonance (NMR) spectra were in all cases consistent with the proposed structures, Characteristic chemical shifts (6) are given in parts per million (ppm) downfield from tetramethylsilane using conventional abbreviations for designation of major peaks: e.g. s, singlet; d, doublet; t, triplet; q, quartet; m, multiplet; br, broad. The mass spectra (m/z) were recorded using either electrospray ionisation (ESI) or atmospheric pressure chemical ionisation (APCI). The following abbreviations have been used for common solvents: CDCl₃, deuterochloroform; D₆-DMSO, hexadeuterodimethylsulphoxide.

### Example 1

### Cis-3-[(8′-chloro-2′-oxo-2′,3′-dihydro-1′H-spiro[cyclohexane-1,4′-quinazolin]-5′-yl)oxy]cyclobutanecarboxylic acid

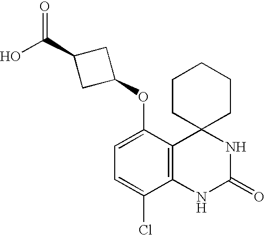

To a solution of the alcohol of Preparation 8 (50 mg, 0.14 mmol) in 99.25:0.75 acetonitrile:water (2 ml) was added a solution of periodic acid (82 mg, 0.359 mmol) and chromium (VI) oxide (1.6 mg, 0.016 mmol) in 99.25:0.75 acetonitrile:water (2 ml), maintaining the reaction temperature below 5° C. The reaction mixture was stirred at room temperature for 18 hours. The reaction mixture was filtered and the residue washed with 99.25:0.75 acetonitrile: water, 2N hydrochloric acid:methanol (5:1), water and methanol. The residue was dried in vacuo to yield the title compound as a white solid (28 mg, 0.077 mmol, 55%).

¹H-NMR (D₆-DMSO, 400 MHz): δ 1.17 (m, 1H), 1.40-1.65 (m, 5H), 1.79 (m, 2H), 2.16 (m, 2H), 2.48 (m, 2H), 2.72 (m, 3H), 4.64 (m, 1H), 6.43 (d, 1H), 7.0 (s, 1H), 7.21 (d, 1H), 7.90 (s, 1H), 12.26 (bs, 1H). LRMS m/z (APCI): 365[M+H]⁺, 406[M+CH₃CN+H]⁺

### Example 2

### Trans-3-[(8′-chloro-2′-oxo-2′,3′-dihydro-1′H-spiro[cyclohexane-1,4′-quinazolin]-5′-yl)oxy]cyclobutanecarboxylic acid

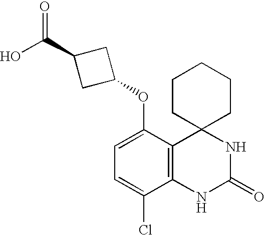

To a solution of the alcohol of Preparation 11 (2.05 g, 5.84 mmol) in acetonitrile containing 0.75% water (50 ml) was added a solution of chromium (VI) oxide (12 mg, 0.11 mmol) and periodic acid (3.33 g, 14.6 mmol) and the reaction mixture stirred at 40° C. for 96 hours. Water (100 ml) was added and the suspension stirred for 2 hours. The resulting precipitate was collected by filtration, washed with water and dried in vacuo to yield the title compound (1.90 g, 5.2 mmol, 89%).

¹H-NMR (D₆-DMSO, 400 MHz): □ 1.2 (m, 1H), 1.2 (m, 2H), 1.6 (m, 2H), 1.8 (m, 2H), 2.3 (m, 2H), 2.6 (m, 2H), 3.1 (m, 1H), 3.2 (s, 1H), 4.0 (bs, 1H), 4.8 (m, 1H), 6.4 (d, 1H), 7.0 (s, 1H), 7.2 (d, 1H), 7.9 (s, 1H). LRMS m/z (APCI) 365 [MH]⁺

### Preparations

### Preparation 1

### 3-[(Benzyloxy)methyl]-2,2-dichlorocyclobutanone

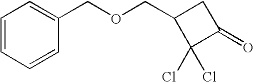

Zinc dust (6.54 g, 0.1 mol) was suspended in water (30 ml) and argon bubbled through the suspension for 15 minutes before the addition of copper (II) sulphate (780 mg, 3.1 mmol). The reaction mixture was stirred at room temperature, under argon for 30 minutes. The mixture was filtered under a stream of argon and the solid washed with water (100 ml), acetone (100 ml) and dried in vacuo for 4 hours. The resultant zinc/copper couple was suspended in diethyl ether: 1,2-dimethoxyethane (70 ml: 10 ml) under argon and allyl benzyl ether (4.6 ml, 30 mmol) added. A solution of trichloroacetyl chloride (9 ml, 81 mmol) in diethyl ether: 1,2-dimethoxyethane (58 ml: 7 ml) was added dropwise over 45 minutes and the reaction mixture heated to reflux for 48 hours. The reaction mixture was filtered through Celite® and the salts washed with diethyl ether (3×70 ml). The filtrate was evaporated in vacuo and the residue redissolved in hexane (150 ml). The remaining solids were removed by filtration and the filtrate washed with a saturated aqueous solution of sodium hydrogen carbonate (2×100 ml), brine (80 ml), dried over magnesium sulphate, filtered and evaporated in vacuo. The crude material was purified by column chromatography over silica gel eluting with 10-25% hexane: diethyl ether. The title compound was obtained as a yellow oil (7.03 g, 27.3 mmol, 91%).

¹H-NMR (CDCl₃, 400 MHz): □ 3.11-3.21 (m, 2H), 3.48 (m, 1H), 3.70 (m, 1H), 3.85 (m, 1H), 7.35 (m, 5H), 4.58 (s, 2H).

### Preparation 2

### 3-[(Benzyloxy)methyl]cyclobutanone

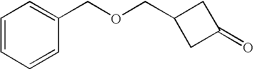

To a solution of the dichlorocyclobutanone of Preparation 1 (5.98 g, 23.08 mmol) in methanol saturated with ammonium chloride (90 ml) was added zinc powder (9.25 g, 142 mmol) and the reaction mixture stirred at room temperature for 2 hours. Ammonium chloride was added and the reaction mixture stirred at room temperature for a further 6 hours. The mixture was filtered through Celite® and the salts washed with diethyl ether (50 ml). The filtrate was concentrated in vacuo and the residue partitioned between diethyl ether (200 ml) and water (100 ml). The mixture was filtered and the organic phase washed with water, dried over magnesium sulphate, filtered and evaporated in vacuo. The title compound was obtained as a yellow oil (3.7 g, 19.5 mmol, 84%).

¹H-NMR (CDCl₃, 400 MHz): □ 2.69 (m, 1H), 2.90 (m, 2H), 3.11 (m, 2H), 3.60 (d, 2H), 4.56 (s, 2H), 7.34 (m, 5H).

### Preparation 3

### Cis-3-[(benzyloxy)methyl]cyclobutanol

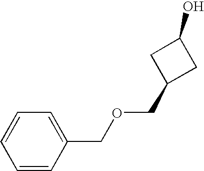

To a solution of the cyclobutanone of Preparation 2 (1.166 g, 6.13 mmol) in tetrahydrofuran stirring at −70° C., was added dropwise a 1M solution of lithium tri-sec-butylborohydride in tetrahydrofuran (40 ml), maintaining the reaction temperature below −65° C. The reaction was allowed to warm to room temperature over 18 hours. The reaction mixture was quenched with a saturated aqueous solution of sodium hydrogen carbonate (25 ml) then cooled to 5° C. 30% Aqueous hydrogen peroxide (4 ml) was added dropwise, maintaining the reaction temperature below 10° C. The mixture was extracted from water into ethyl acetate (50 ml) and the combined organic phases washed with brine (30 ml), dried over magnesium sulphate, filtered and evaporated in vacuo. The crude material was purified by column chromatography over silica gel eluting with 25-50% ethyl acetate: pentane to yield a colourless oil (1.05 g, 5.5 mmol, 89%). ¹H-NMR indicated that a 15:1 ratio of cis:trans isomers had been obtained.

¹H-NMR (CDCl₃, 400 MHz): □ 1.70 (m, 2H), 2.10 (m, 1H), 2.46 (m, 2H), 3.45 (d, 2H), 4.15 (q, 1H), 4.52 (s, 2H), 7.33 (m, 5H).

### Preparation 4

### Trans-3-[(benzyloxy)methyl]cyclobutyl 4-nitrobenzoate

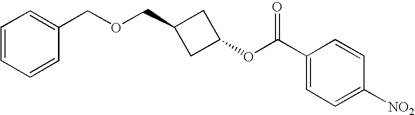

A solution of diethyl azodicarboxylate (2 g, 11.5 mmol) in tetrahydrofuran (5 ml) was added dropwise to a stirred solution of the cyclobutyl alcohol of Preparation 3 (1.05 g, 5.47 mmol), 4-nitrobenzoic acid (1.82 g, 10.9 mmol) and triphenylphosphine (3.016 g, 11.5 mmol) in tetrahydrofuran (20 ml) at 0° C. The reaction mixture was stirred at room temperature for 18 hours. The solvent was evaporated in vacuo and the residue redissolved in diethyl ether (30 ml). The remaining solid was removed by filtration and the filtrate evaporated in vacuo. The crude material was purified by column chromatography over silica gel eluting with 1:10 to 1:3 ethyl acetate: pentane to yield a colourless oil (1.64 g, 4.8 mmol, 88%). ¹H-NMR indicated that a 15:1 ratio of trans:cis isomers had been obtained.

¹H-NMR (CDCl₃, 400 MHz): δ 2.40 (m, 4H), 2.67 (m, 1H), 3.53 (d, 2H), 4.57 (s, 2H), 5.36 (q, 1H), 7.37 (m, 5H), 8.20 (d, 2H), 8.29 (d, 2H).

### Preparation 5

### Trans-3-[(benzyloxy)methyl]cyclobutanol

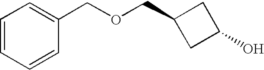

To a solution of the p-nitroester of Preparation 4 (1.64 g, 4.8 mmol) in 1,4-dioxane (35 ml) was added a solution of sodium hydroxide (385 mg, 9.6 mmol) in water (25 ml) and the reaction mixture stirred at room temperature for 30 minutes. Acetic acid (0.4 ml, 7 mmol) was added and the mixture concentrated in vacuo. The residue was extracted from a saturated aqueous solution of sodium hydrogen carbonate into ethyl acetate (20 ml), dried over magnesium sulphate, filtered and evaporated in vacuo. The title compound was obtained as a yellow oil (850 mg, 4.4 mmol, 92%).

¹H-NMR (CDCl₃, 400 MHz): δ 2.08 (m, 2H), 2.20 (m, 2H), 2.47 (m, 1H), 3.47 (d, 2H), 4.39 (q, 1H), 4.52 (s, 2H), 7.34 (m, 5H).

### Preparation 6

### Trans-3-[(benzyloxy)methyl]cyclobutyl p-toluenesulphonate

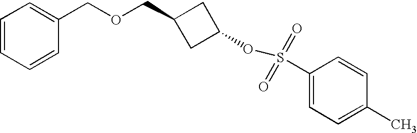

p-Toluenesulphonyl chloride (1.18 g, 6.2 mmol) was added portionwise to a stirred solution of the cyclobutanol of Preparation 5 (850 mg, 4.42 mmol) in pyridine (5 ml) at 0° C. and the reaction mixture stirred at room temperature for 18 hours. The solvent was concentrated in vacuo and the residue redissolved in ethyl acetate (30 ml), washed with 2N hydrochloric acid, (30 ml) a saturated aqueous solution of sodium hydrogen carbonate (30 ml), brine (30 ml), dried over magnesium sulphate, filtered and evaporated in vacuo. The crude material was purified by column chromatography over silica gel eluting with dichloromethane. The title compound was obtained as a colourless oil (1.53 g, 4.4 mmol).

¹H-NMR (CDCl₃, 400 MHz): δ 2.15 (m, 2H), 2.31 (m, 2H), 2.44 (s, 3H), 2.49 (m, 1H), 3.4 (d, 2H), 4.49 (s, 2H), 4.93 (q, 1H), 7.32 (m, 7H), 7.75 (d, 2H).

### Preparation 7

### 5′-({Cis-3-[(benzyloxy)methyl]cyclobutyl}oxy)-8′-chloro-1′H-spiro[cyclohexane-114′-quinazolin]-2′(3′H)-one

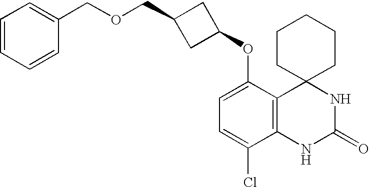

8′-Chloro-5′-hydroxy-1′H-spiro[cyclohexane-1,4′-quinazolin]-2′(3′H)-one (prepared as described in Bioorg. Med. Chem. Lett, (2004), 14 (18), 4627-4632) (640 mg, 2.4 mmol), potassium carbonate (400 mg, 2.9 mmol) and 18-crown-6 (767 mg, 2.9 mmol) were combined in dimethylformamide (8 ml) and the reaction mixture heated to 80° C. A solution of the tosylate of Preparation 6 (1 g, 2.9 mmol) in dimethylformamide was added in 3 portions and the mixture heated at 80° C. for a further 18 hours. The reaction mixture was partitioned between ethyl acetate (100 ml) and water (150 ml) and the solid collected by filtration. The phases were separated and the aqueous phase reextracted with ethyl acetate, diluted with brine and again extracted into ethyl acetate. The combined organic phases were concentrated in vacuo and the residue triturated with water and methanol. The combined crude products were purified by column chromatography over silica gel eluting with dichloromethane to dichloromethane: ethyl acetate (1:1) to obtain the title compound as an off-white solid (685 mg, 1.156 mmol, 64%).

¹H-NMR (D₆-DMSO, 400 MHz): δ 1.1 (m, 1H), 1.4 (m, 2H), 1.6 (m, 3H), 1.7 (m, 2H), 1.8 (m, 2H), 2.3 (m, 1H), 2.5 (m, 4H), 3.4 (s, 2H), 4.4 (s, 2H), 4.6 (m, 1H), 6.4 (d, 1H), 7.0 (s, 1H), 7.2 (d, 1H), 7.3 (m, 5H), 7.8 (s, 1H).

### Preparation 8

### 8′-Chloro-5′-{[cis-3-(hydroxymethyl)cyclobutyl]oxy}-1′H-spiro[cyclohexane-1,4′-quinazolin]-2′(3′H)-one

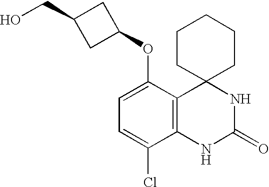

A 2M solution of boron trichloride-dimethyl sulfide complex in dichloromethane (1.8 ml, 3.6 mmol) was added to a suspension of the benzyl alcohol of Preparation 7 (400 mg, 0.9 mmol) in dichloromethane (10 ml) and the reaction mixture stirred at room temperature overnight. A saturated aqueous solution of sodium hydrogen carbonate (10 ml) was added and the mixture stirred for 5 minutes. Dichloromethane and water were added and the resultant solid collected by filtration. The title compound was obtained as a white solid (230 mg, 0.657 mmol, 73%).

¹H-NMR (D₆-DMSO, 400 MHz): δ 1.17 (m, 1H), 1.42 (m, 2H), 1.57 (m, 3H), 1.82 (m, 4H), 2.05 (m, 1H), 2.45 (m, 4H), 3.38 (t, 2H), 4.58 (m, 2H), 6.41 (d, 1H), 6.99 (s, 1H), 7.20 (d, 1H), 7.86 (s, 1H). LRMS m/z (APCI) 351 [MH]⁺

### Preparation 9

### Cis-3-[(benzyloxy)methyl]cyclobutyl p-toluenesulphonate

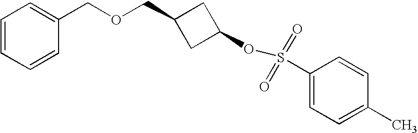

Pyridine (14.3 ml, 176 mmol) and p-toluenesulphonyl chloride (20.2 g, 105.9 mmol) were added to a solution of the alcohol of Preparation 3 (17 g, 88.4 mmol) in dichloromethane (90 ml) stirring at 5° C. and the reaction mixture was stirred at room temperature for 18 hours. The reaction mixture was diluted with dichloromethane (50 ml), washed with 2N hydrochloric acid (50 ml), a saturated aqueous solution of sodium hydrogen carbonate (50 ml), dried over magnesium sulphate, filtered and evaporated in vacuo. The crude material was purified by column chromatography over silica gel eluting with pentane: ethyl acetate (19:1, 9:1, 4:1). The title compound was obtained as a colourless oil (24.8 g, 71.6 mmol, 81%).

¹H-NMR (CDCl₃, 400 MHz): δ 1.95 (m, 2H), 2.1 (m, 1H), 2.35 (m, 2H), 2.45 (s, 3H), 3.4 (m, 2H), 4.5 (s, 2H), 4.7 (m, 1H), 7.3 (m, 7H), 7.8 (m, 2H). LRMS m/z (ESI) 347 [MH]⁺

### Preparation 10

### 5′-({Trans-3-[(benzyloxy)methyl]cyclobutyl}oxy)-8′-chloro-1′H-spiro[cyclohexane-1,4′-quinazolin]-2′(3′H)-one

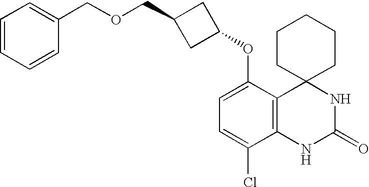

### Method A

Caesium carbonate (730 mg, 2.24 mmol) was added to a stirred suspension of 8′-chloro-5′-hydroxy-1′H-spiro[cyclohexane-1,4′-quinazolin]-2′(3′H)-one (500 mg, 1.87 mmol) in dimethylformamide (2 ml) and the reaction mixture heated to 80° C. After 5 minutes a solution of the tosylate of Preparation 9 (710 mg, 2.05 mmol) in dimethylformamide (1 ml) was added and the reaction mixture heated at 80° C. for 18 hours. The mixture was extracted from brine (60 ml) into ethyl acetate (1×80 ml, 2×30 ml), washed with brine (3×100 ml), dried over magnesium sulphate, filtered and evaporated in vacuo. The title compound was obtained as a slightly impure cream solid (800 mg, 0.96 mmol, 96%).

### Method B

To a solution of 8′-chloro-5′-hydroxy-1′H-spiro[cyclohexane-1,4′-quinazolin]-2′(3′H)-one (950 mg, 3.56 mmol) in dimethylformamide (12 ml) stirring at 80° C. was added potassium carbonate (590 mg, 4.27 mmol) and 18-crown-6 (1.1 g, 4.27 mmol). The reaction mixture was stirred for 10 minutes before the addition of a solution of the tosylate of Preparation 9 (1.48 g, 4.27 mmol) in dimethylformamide (3 ml). The reaction mixture was heated at 80° C. for 24 hours. The mixture was poured onto water:methanol (75 ml: 25 ml), stirred for 10 minutes and the resulting precipitate collected by filtration and washed with methanol. The solid was dissolved in dichloromethane, filtered through Celite® and the resulting filtrate evaporated in vacuo to yield the title compound as a 9:1 mixture of trans:cis isomers (887 mg, 2.0 mmol, 56%).

¹H-NMR (CDCl₃, 400 MHz): δ 1.3 (m, 1H), 1.5-1.9 (m, 9H), 2.4 (m, 3H), 2.6 (m, 2H), 3.5 (d, 2H), 4.6 (s, 2H), 4.75 (m, 1H), 5.85 (bs, 1H), 6.25 (d, 1H), 7.05 (bs, 1H), 7.1 (d, 1H), 7.3-7.4 (m, 5H). LRMS m/z (ESI) 441 [MH]⁺

### Preparation 11

### 8′-Chloro-5′-{[trans-3-(hydroxymethyl)cyclobutyl]oxy}-1′H-spiro[cyclohexane-1,4′-quinazolin]-2′(3′H)-one

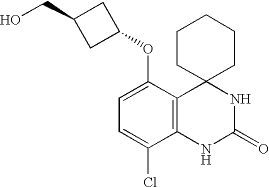

A 2M solution of boron trichloride-dimethyl sulfide complex in dichloromethane (15 ml) was added dropwise to a solution of the benzyl ether of Preparation 10 (3.5 g, 7.9 mmol) in dichloromethane (80 ml) and the reaction mixture stirred at room temperature for 18 hours. The mixture was poured into a saturated aqueous solution of sodium hydrogen carbonate (200 ml) and stirred until the effervescence ceased. The mixture was extracted into dichloromethane (1×200 ml, 2×100 ml), washed with brine (50 ml), dried over magnesium sulphate, filtered and evaporated in vacuo. The crude material was recrystallised from acetonitrile to yield the title compound as a 91:9 ratio of trans:cis products (2.33 g, 6.65 mmol, 84%).

¹H-NMR (CDCl₃, 400 MHz): δ 1.3 (m, 1H), 1.5 (m, 2H), 1.8 (m, 5H), 2.4 (m, 4H), 2.6 (m, 3H), 3.8 (d, 2H), 4.8 (m, 1H), 5.7 (bs, 1H), 6.25 (d, 1H), 7.0 (bs, 1H), 7.1 (d, 1H). LRMS m/z (ESI) 351 [MH]⁺

### Assay of Examples 1 and 2

The ability of the compounds of formula (IV) to inhibit PDE7 may be measured using the following assay protocol.

PDE7A and PDE7B enzymes catalyse the hydrolysis of 3′,5′-cyclic adenosine monophosphate (cAMP) to the 5′adenosine monophosphate, 5′AMP. In a multiwell plate, PDE enzyme, [³H]-cAMP and the tested compounds, are incubated at room temperature. The incubation is terminated by addition of commercially available yttrium silicate scintillation proximity assay (SPA) beads containing zinc sulphate. The yttrium silicate beads preferentially bind linear nucleotides, thus the product of the enzyme reaction, [³H]-5′AMP binds to the bead to produce a light signal, which is detected by a scintillation counter. The amount of signal produced directly correlates with the amount of product formed, and thus the activity of the enzyme. The maximum signal is obtained where enzyme and substrate are incubated alone. The background signal is measured from wells either containing no enzyme, or from wells containing a supra-maximal concentration of a known PDE7A/B inhibitor. Each purified batch of enzyme is quality controlled and its Kₘ, Vₘₐₓ and specific activity determined from kinetic studies before use in compound inhibition studies. The inhibition of the enzyme, by a test compound, is calculated relative to the maximum and background responses. Using these data a % inhibition value is calculated relative to the maximum and minimum values obtained.

### Preparation of Working Solutions

A 1000 ml stock of buffer was prepared from the following ingredients:

| | | Final | Stock Soln. | ml/1000 |
|---|---|---|---|---|
| Reagent | Source | concentration | concentration | ml |
| HEPES (buffer) | Sigma | 50 mM | 1 | 50 |
| MgCl₂ | Sigma | 5 mM | 1 | 5 |
| Pluronic ® | Sigma | 0.025% | 5% | 5 |
| (detergent) | | | | |
| Millipore ® 18 mΩ | Milli- | | | 940 |
| purified water | pore | | | |

The stock buffer was adjusted to pH 7.4 at room temperature and then filtered through a 0.2 μm filter. The stock buffer is stable at 4° C. for 1 month from the date of preparation.

On the day of experiment, Bovine Serum Albumin (BSA, available from Sigma) was added to the required volume of buffer to create a 0.00625% BSA final solution. This was achieved by preparing a stock 10% BSA solution as follows:

### Preparation of Stock 10% BSA Solution

1 g BSA was dissolved in 10 ml purified water, mixed by inversion to ensure homogeneity and aliquot in 100 μl volumes in appropriately labelled tubes. The 10% BSA solution is stable at −20° C. for up to 6 months.

An aliquot of the stock 10% BSA stock solution was removed from storage and allowed to thaw out at room temperature before being used to create the BSA working solution as follows:

### Preparation of 10 ml Working BSA Assay Buffer

| | | Final BSA |
|---|---|---|
| Reagent | Volume | concentration |
| 1x Buffer stock | 9.99 ml | |
| 10% BSA stock | 6.25 μl | 0.00625% |

### Preparation of Standard Compound and Controls

The compound of Example 75 of WO 02/074754, 5′-carboxypropoxy-8′-chloro-spiro[cyclohexane-1-4′-(3′,4′-dihydro)quinazolin]-2′(1′H)-one (hereinafter “Compound A”) was used as a standard.

4 mM stock solution prepared in 100% DMSO can be stored at 4° C. The volume of DMSO can be calculated as follows:

$$Volume of DMSO ( ml ) = \frac{weight of compound}{Molecular weight of compound} \times 250$$

The 30× Max control is a solution of 100% DMSO. The 30×Min control is achieved using a 30 μM of Compound A in 100% DMSO to yield no enzyme activity. 5 ml of a 30 μM solution of Compound A can be prepared by adding 4.962 ml of 100% DMSO to 37.5 μl of 4 mM Compound A.

### Method

On the day of assay, the 1× final assay buffer was prepared as detailed previously and kept on ice until needed.

### Kinetic Studies

For each new batch of enzyme, the Kₘ was determined, and the amount of enzyme required to obtain ˜1000 cpm signal in 45 minutes, whilst remaining in the linear portion of the reaction progress curve, was assessed. Ideally <10% of available [³H]-cAMP will be hydrolysed during the course of the assay.

### Enzyme Solution

The optimisation of this assay has been carried out using cell lysate containing full length PDE7A and PDE7B enzyme. The concentration of the enzyme in this cell lysate sample is unknown, so the specific activity of the cell lysate is used as a measure to ensure that the same activity per well is used despite any batch-to-batch variation of concentration/activity.

### Preparation of PDE7A/B Enzyme

PDE7 stock enzyme was prepared and kept at −20° C. in appropriately sized aliquots to reduce the number of freeze/thaw cycles. The following table shows the volumes required to make 9 mls of PDE7A/B enzyme solution. PDE7A is diluted to 1/8000 and PDE7B to 1/10000.

| | | | | Overall |
|---|---|---|---|---|
| | | Vol. of PDE7 | Vol. of | Dilution of |
| | | stock/diluted | Buffer + | Enzyme |
| Enzyme | Dilution | soln (μl) | BSA (μl) | stock |
| PDE7A | PDE7B 1:100 | 5 | 495 | 1:100 |
| | dilution of stock | | | |
| | 1:40 dilution of | 25 | 975 | 1:4000 |
| | above solution | | | |
| | This enzyme solution is further diluted when all the assay | | | |
| | components are dispensed into the assay plate i.e. 14 μl | | | |
| | enzyme solution is dispensed into a total assay volume of | | | |
| | 30 μl, giving an overall 1/8000-enzyme dilution. | | | |
| PDE7B | PDE7B 1:100 | 5 | 495 | 1:100 |
| | dilution of stock | | | |
| | 1:50 dilution of | 20 | 980 | 1:5000 |
| | above solution | | | |

This enzyme solution is further diluted when all the assay
components are dispensed into the assay plate i.e. 14 μl
enzyme solution is dispensed into a total assay volume of
30 μl, giving an overall 1/10000-enzyme dilution.

Once the enzyme solution was prepared it was kept on ice prior to usage.

Preparation of 50 nM Adenosine 3′, 5′ Cyclic Phosphate (cAMP) Substrate Solution

The substrate is composed of a mixture of unlabelled cAMP and cAMP radiolabelled with tritium ([³H]-cAMP). The specifications of the stock of [³H]-cAMP will determine the volumes used.

The preparation of 9 ml of substrate solution using a [³H]-cAMP stock which is 1 mCi/ml and 24 Ci/mmol (therefore 41.66 μM) is described below:

Kₘ for the enzymes batches to date is as follows:

PDE7A—20 nM PDE7B—100 nM

The assay requires 15 μl of substrate solution to be dispensed into a total assay volume of 30 μl, ie a ×2 dilution in the assay plate occurs.

The final assay [cAMP] of ˜25 nM is required, so ˜50 nM [³H]-CAMP was prepared.

9 ml of substrate solution was prepared by mixing 10.8 μl of [³H]-CAMP (available from Amersham) with 8975 μl of assay buffer.

The exact concentration of cAMP was determined by taking 3 samples of 15 μl into scintillation vials. 4 ml Starscint® (a scintillation cocktail, available from Perkin Elmer), was then added and the tubes counted on a β-counter on a dpm program.

The concentration of radioligand is determined by the following equation:

$$[ Radioligand ] ( M ) = \frac{DPM}{{( 2.22 \times {10}^{12} )}_{( DPM / Ci )} \times {( specific activity )}_{( Ci / Mol )} \times {( volume of sample )}_{( L )}}$$

The concentration is then divided by 2 to allow for the ×2 dilution occurring in the assay plate.

Preparation of 6.6 mg/ml Yttrium Silicate PDE SPA Beads

Phosphodiesterase SPA beads (Yttrium Silicate) are available from Amersham.

Following the manufacturer's recommendations the vial of beads was reconstituted using 28 ml distilled or deionised water (˜20 mg/ml). The reconstituted beads are stable for 1 month when stored at 2-8° C. To prepare the beads for the assay, the reconstituted beads were diluted 3-fold in sterile double distilled water (˜6.6 mg/ml). The beads can settle, so were constantly stirred/agitated whilst dispensing.

30 μl of the ˜6.6 mg/ml beads are added to the 30 μl assay, giving a final bead concentration of ˜0.2 mg/well.

Compound dilutions and “background” wells were made 30 stronger than required in the assay plate to allow for 1 μl compound to be diluted by 29 μl of other assay components (14 μl enzyme and 15 μl radioligand). Thus for a final assay concentration of 10 μM, the compound must be at 300 μM in the compound addition plate. 4 mM stocks of compound are supplied in 100% DMSO (or are made up @ 4 mM from powder submissions). This requires 1/13.33 dilution in DMSO to be made.

### Assay Protocol

1 μl test compound was transferred into a suitable multi-well assay plate immediately prior to reagent assay addition, 14 μl enzyme solution was then added to the assay plate, followed by 15 μl substrate solution (ie: final assay volume 30 μl, with a final screening compound concentration of 1 μM). The plate was then sealed using a plate sealer and incubated at room temperature for 45 min on the plate shaker.

30 μl Yttrium Silicate PDE4 SPA beads were then added, ensuring constant stirring of the beads to give even distribution in the assay plate. The plate was then sealed using a plate sealer and incubated at room temperature for 30mins on the plate shaker. The beads were then allowed to settle for 30mins, before spinning the plates for 1 min at 200 g.

The plates were then read on a suitable radioactive counter, for example NXT-TopCount™ (available from Perkin Elmer) using the relevant protocol (30 second read time per well).

The data was fitted to a sigmoid curve using a least squares algorithm.

The IC₅₀ value was converted to a Kᵢ value using the Cheng-Prussof equation:

$${K}_{i} = \frac{{IC}_{50}}{1 + \underline{[ radioligand ]}}$$

The PDE7 inhibitory activity of the compounds of the present invention was tested according to the above protocol. The Kᵢ values obtained are as follows:

| Example No | Kᵢ PDE7A (nM) | Kᵢ PDE7B (nM) |
|---|---|---|
| 1 | 1.9 | 4.6 |
| 2 | 3.1 | 13.4 |

### Example 3

The following example illustrates the embodiments and principles of the invention and comprises the use of a potent and selective inhibitor of the PDE7 5′-(3-(Carboxy)propoxy)-8′-chlorospiro[cyclohexane-1,4′-quinazolin]-2′(1′H)-one. The structure of inhibitor 5′-(3-(Carboxy)propoxy)-8′-chlorospiro[cyclohexane-1,4′-quinazolin]-2′(1′H)-one is:

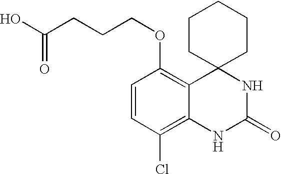

### Assay of Example 3

### Animals for In Vivo Models

Male Sprague Dawley rats weighing 150-400 g obtained from Charles River (Manston, Kent, UK.) were housed in groups of 4. All animals were kept under a 12 h light/dark cycle (lights on at 07 h 00 min) with food and water ad libitum. All experiments were carried out by an observer blind to the treatments and in accordance with the Home Office Animals (Scientific Procedures) Act 1986.

### Chronic Constriction Injury (CCI) Rat Model of Neuropathic Pain

The CCI of sciatic nerve was performed as previously described by Bennett and Xie (Bennett G J, Xie Y K. A peripheral mononeuropathy in rat that produces disorders of pain sensation like those seen in man. Pain: 33:87-107, 1988). Animals were anaesthetised with a 2% isofluorane/O₂ mixture. The right hind thigh was shaved and swabbed with 1% iodine. Animals were then transferred to a homeothermic blanket for the duration of the procedure and anaesthesia maintained during surgery via a nose cone. The skin was cut along the line of the thighbone. The common sciatic nerve was exposed at the middle of the thigh by blunt dissection through biceps femoris. About 7 mm of nerve was freed proximal to the sciatic trifurcation, by inserting forceps under the nerve and the nerve gently lifted out of the thigh. Suture was pulled under the nerve using forceps and tied in a simple knot until slight resistance was felt and then double knotted. The procedure was repeated until 4 ligatures (4-0 silk) were tied loosely around the nerve with approx 1 mm spacing. The incision was closed in layers.

### Assessment of Static and Dynamic Allodynia in the Rat

### Static Allodynia.

Animals were habituated to wire bottom test cages prior to the assessment of allodynia. Static allodynia was evaluated by application of von Frey hairs (Stoelting, Wood Dale, Ill., USA.) in ascending order of force (0.6, 1, 1.4, 2, 4, 6, 8, 10, 15 and 26 grams) to the plantar surface of hind paws. Each von Frey hair was applied to the paw for a maximum of 6 sec, or until a withdrawal response occurred. Once a withdrawal response to a von Frey hair was established, the paw was re-tested, starting with the filament below the one that produced a withdrawal, and subsequently with the remaining filaments in descending force sequence until no withdrawal occurred. The highest force of 26 g lifted the paw as well as eliciting a response, thus represented the cut off point. Each animal had both hind paws tested in this manner. The lowest amount of force required to elicit a response was recorded as paw withdrawal threshold (PWT) in grams. Static allodynia was defined as present if animals responded to a stimulus of, or less than, 4 g, which is innocuous in naive rats (Field M J, Bramwell S, Hughes J, Singh L. Detection of static and dynamic components of mechanical allodynia in rat models of neuropathic pain: are they signalled by distinct primary sensory neurones? Pain, 1999; 83:303-11).

### Dynamic Allodynia

Dynamic allodynia was assessed by lightly stroking the plantar surface of the hind paw with a cotton bud. To avoid recording general motor activity, care was taken to perform this procedure in fully habituated rats that were not active. At least two measurements were taken at each time point, the mean of which represented the paw withdrawal latency (PWL). If no reaction was exhibited within 15 sec the procedure was terminated and animals were assigned this withdrawal time. A pain withdrawal response was often accompanied with repeated flinching or licking of the paw. Dynamic allodynia was considered to be present if animals responded to the cotton stimulus within 8 sec of commencing stroking (Field et al, 1999).

### Data Analysis

All the experiments were conducted in blind. When the experiment was carried out in more then one day and where technically possible, all groups occurred on each day with equal replication. Static allodynia was expressed as median [LQ; UQ] and analysed by Mann Whitney U test. Dynamic allodynia was expressed as arithmetic mean±SEM and analysed by ANOVA.

### Effect of 5′-(3-(Carboxy)propoxy)-8′-chlorospiro[cyclohexane-1,4′-quinazolin]-2′(1′H)-one on CCI-Induced Static and Dynamic Allodynia

Naïve rats exhibit paw withdrawal thresholds of approximately log to von Frey application and find application of a cotton bud stimulus completely innocuous. Following nerve injury rats display increased sensitivity to both of these stimuli indicating the development of static and dynamic allodynia. From 14 days post surgery animals exhibited typical static and dynamic allodynic responses and the baseline recorded before the test were <4 g and <4 sec, respectively in all animals. These allodynic responses remained consistent throughout the experiments in the vehicle-treated group. Following oral (PO) administration, 5′-(3-(Carboxy)propoxy)-8′-chlorospiro[cyclohexane-1,4′-quinazolin]-2′(1′H)-one (0.3, 1 and 3 mg/kg) reversed the maintenance of CCI-induced static and dynamic allodynia in a dose dependent manner (FIG. 1A and FIG. 1B). The MED for static and dynamic allodynia were 1 mg/kg and 3 mg/kg respectively and both end points produced a peak effect at 1 hr post administration. The highest dose showed an anti-allodynic effect in both behavioral tests from 30 min post dose (p<0.01 vs vehicle-treated group). It reversed static allodynia with a curve profile comparable to gabapentin (100 mg/kg, PO) while its effect in dynamic allodynia is less potent but significantly different from vehicle treated CCI rats (10.2±1.4 vs 3.7±0.7 at 1 hrs post administration).

1. Use of a PDE7 inhibitor for the manufacture of a medicament for the treatment of neuropathic pain.

2. Use as claimed in claim 1 wherein the PDE7 inhibitor is a selective PDE7 inhibitor.

3. Use as claimed in claim 1 or claim 2 wherein the PDE7 inhibitor is a compound having the following formula (I), (II) or (III), 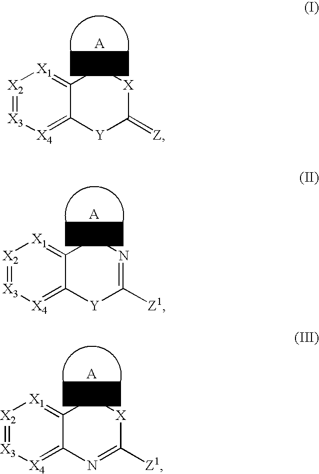
in which,
a) X₁, X₂, X₃ and X₄ are the same or different and are selected from:
N, provided that not more than two of the groups X₁, X₂, X₃ and X₄ simultaneously represent a nitrogen atom, or,
C—R¹, in which R¹ is selected from:
Q1, or
lower alkyl, lower alkenyl or lower alkynyl, these groups being unsubstituted or substituted with one or several groups Q2;
the group X⁵—R⁵ in which,
X⁵ is selected from:
a single bond,
lower alkylene, lower alkenylene or lower alkynylene, optionally interrupted with 1 or 2 heteroatoms chosen from O, S, S(═O), SO₂ or N, the carbon atoms of these groups being unsubstituted or substituted with one or several groups, identical or different, selected from SR⁶, OR⁶, NR⁶R⁷, ═O, ═S or ═N—R⁶ in which R⁶ and R⁷ are the same or different and are selected from hydrogen or lower alkyl, and,
R⁵ is selected from aryl, heteroaryl, cycloalkyl optionally interrupted with C(═O) or with 1, 2, or 3 heteroatoms chosen from O, S, S(═O), SO₂ or N, cycloalkenyl optionally interrupted with C(═O) or with 1, 2, or 3 heteroatoms chosen from O, S, S(═O), SO₂ or N, or a bicyclic group, these groups being unsubstituted or substituted with one or several groups selected from Q3, heteroaryl or lower alkyl optionally substituted with Q3;
in which Q1, Q2, Q3 are the same or different and are selected from
hydrogen, halogen, CN, NO₂, SO₃H, P(═O)(OH)₂
OR², OC(═O)R², C(═O)OR², SR², S(═O)R², C(═O)—NH—SO₂—CH₃, NR³R⁴, Q-R², Q-NR³R⁴, NR²-Q-NR³R⁴ or NR³-Q-R² in which Q is selected from C(═NR), C(═O), C(═S) or SO₂, R is selected from hydrogen, CN, SO₂NH₂ or lower alkyl and R², R³ and R⁴ are the same or different and are selected from:
hydrogen,
lower alkyl optionally interrupted with C(═O), Q4-aryl, Q4-heteroaryl, Q4-cycloalkyl optionally interrupted with C(═O) or with 1 or 2 heteroatoms chosen from O, S, S(═O), SO₂ or N, or Q4-cycloalkenyl optionally interrupted with C(═O) or with 1 or 2 heteroatoms chosen from O, S, S(═O), SO₂ or N, in which
Q4 is selected from (CH₂)ₙ, lower alkyl interrupted with one heteroatom selected from O, S or N, lower alkenyl or lower alkynyl, these groups being optionally substituted with lower alkyl, OR′ or NR′R″ in which R′ and R″ are the same or different and are selected from hydrogen or lower lower alkyl;
n is an integer selected from 0, 1, 2, 3 or 4;
these groups being unsubstituted or substituted with one or several groups selected from lower alkyl, halogen, CN, CH₃, SO₃H, SO₂CH₃, C(═O)—NH—SO₂—CH₃, CF₃, OR⁶, COOR₆, C(═O)R⁶, NR³R⁷, NR C(═O)R⁷, C(═O)NR⁶R⁷ or SO₂NR⁶R⁷, in which R³ and R⁷ are the same or different and are selected from hydrogen or lower alkyl optionally substituted with one or two groups selected from OR, COOR or NRR⁸ in which R and R⁸ are hydrogen or lower alkyl, and,
R⁶ and R⁷, and/or, R³ and R⁴, together with the nitrogen atom to which they are linked, can form a 4- to 8-membered heterocyclic ring, which may contain one or two heteroatoms selected from O, S, S(═O), SO₂, or N, and which may be substituted with,
(CH₂)ᵣ-Q5, in which n is an integer selected from 0, 1, 2 and 3, and Q5 is a 4- to 8-membered heterocyclic ring which may contain one or two heteroatoms selected from O, S or N and which may be substituted with a lower alkyl, or,
a lower alkyl optionally substituted with OR′, NR′R″, C(═O)NR′R″ or COOR′ in which R′ and R″ are the same or different and are selected from,
H, or,
lower alkyl optionally substituted with OR or COOR in which R is hydrogen or lower alkyl and,
R′ and R″ together with the nitrogen atom to which they are linked, can form a 4- to 8-membered heterocyclic ring, which may contain one or two heteroatoms selected from O, S or N; or,
when X₁ and X₂ both represent C—R¹, the 2 substituents R¹ may form together with the carbon atoms to which they are attached, a 5-membered heterocyclic ring comprising a nitrogen atom and optionally a second heteroatom selected from O, S or N;
b) X is O or NR⁹, in which R⁹ is selected from,
hydrogen, CN, OH, NH₂,
lower alkyl, lower alkenyl or lower alkynyl, these groups being unsubstituted or substituted with cycloalkyl optionally interrupted with 1 or 2 heteroatoms chosen from O, S, S(═O), SO₂ or N, cycloalkenyl optionally interrupted with 1 or 2 heteroatoms chosen from O, S, S(═O), SO₂ or N, aryl, heteroaryl, OR¹⁰, COOR¹⁰ or NR¹⁰R¹¹ in which R¹⁰ and R¹¹ are the same or different and are selected from hydrogen or lower alkyl;
c) Y is selected from O, S or N—R¹², in which R¹² is selected from:
hydrogen, CN, OH, NH₂,
lower alkyl, lower alkenyl or lower alkynyl, these groups being unsubstituted or substituted with, cycloalkyl optionally interrupted with 1 or 2 heteroatoms chosen from O, S, S(═O), SO₂ or N, cycloalkenyl optionally interrupted with 1 or 2 heteroatoms chosen from O, S, S(═O), SO₂ or N, aryl, heteroaryl, OR¹⁰, COOR¹⁰ or NR¹⁰R¹¹ in which R¹⁰ and R¹¹ are the same or different and are selected from hydrogen or lower alkyl;
d) Z is chosen from CH—NO₂, O, S or NR¹³ in which R¹³ is selected from:
hydrogen, CN, OH, NH₂, aryl, heteroaryl, cycloalkyl optionally interrupted with one or several heteroatoms chosen from O, S, S(═O), SO₂ or N, cycloalkenyl optionally interrupted with one or several heteroatoms chosen from O, S, S(═O), SO₂ or N, C(═O)R¹⁴, C(═O)NR¹⁴R¹⁵, OR¹⁴, or,
lower alkyl, unsubstituted or substituted with one or several groups which are the same or different and which are selected OR¹⁴, COOR¹⁰ or NR¹⁴R¹⁵;
R¹⁴ and R¹⁵ being independently selected from hydrogen or lower alkyl, or, R¹⁴ and R¹⁵, together with the nitrogen atom to which they are linked, can form a 4- to 8-membered heterocyclic ring which may contain one or two heteroatoms chosen from O, S or N, and which may be substituted with a lower alkyl, or,
when Y is N—R¹² and Z is N—R¹³, may form together a —CH═N— group or a —C═C— group,
when X is N—R⁹ and Z is N—R¹³, R⁹ and R¹³ may form together a —CH═N— group or a —C═C— group;
e) Z¹ is chosen from H, CH₃ or NR¹⁶R¹⁷ in which R¹⁶ and R¹⁷ are the same or different and are selected from:
hydrogen, CN, aryl, heteroaryl, cycloalkyl optionally interrupted with one or several heteroatoms chosen from O, S, S(═O), SO₂ or N, cycloalkenyl optionally interrupted with one or several heteroatoms chosen from O, S, S(═O), SO₂ or N, C(═O)R¹⁴, C(═O)NR¹⁴R¹⁵OR¹⁴ or,
lower alkyl unsubstituted or substituted with one or several groups selected from OR¹⁴, COOR¹⁴ or NR¹⁴R¹⁵
R¹⁴ and R¹⁵ being chosen from hydrogen or lower alkyl, and,
R¹⁴ and R¹⁵, and/or, R¹⁶ and R¹⁷, together with the nitrogen atom to which they are linked, can form a 4- to 8-membered heterocyclic ring which may contain one or two heteroatoms chosen from O, S or N, and which may be substituted with a lower alkyl;
f) A is a cycle chosen from: 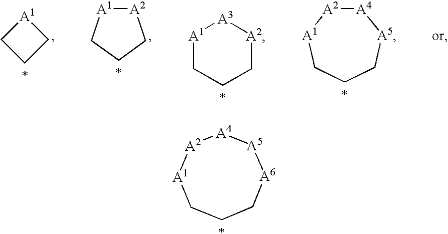
in which,
A¹, A², A⁴, A⁵ and A⁶ are the same or different and are selected from O, S, C, C(═O), SO, SO₂ or N—R¹¹ in which R¹⁵ is selected from:
hydrogen, aryl, heteroaryl, cycloalkyl optionally interrupted with one or several heteroatoms chosen from O, S, S(═O), SO₂ or N, cycloalkenyl optionally interrupted with one or several heteroatoms chosen from O, S, S(═O), SO₂ or N,
lower alkyl unsubstituted or substituted with aryl, heteroaryl, cycloalkyl optionally interrupted with one or several heteroatoms chosen from O, S, S(═O), SO₂ or N, cycloalkenyl optionally interrupted with one or several heteroatoms chosen from O, S, S(═O), SO₂ or N, CN, NR¹⁹R²⁰, C(═O)NR¹⁹R²⁰, OR¹⁹, C(═O)R¹⁹ or C(═O)OR¹⁹ in which R¹⁹ and R²⁰ are identical or different and are selected from hydrogen or lower alkyl;
A³ is selected from O, S, C, C(═O), SO or SO₂, or N—R¹⁸ when A¹ and/or A² are C(═O) or when Y is O or S, wherein R¹⁸ is as defined above;
* represents the carbon atom which is shared between the cycle A and the backbone cycle containing X and/or Y;
each carbon atom of the cycle A is unsubstituted or substituted with 1 or 2 groups, identical or different, selected from lower alkyl optionally substituted with OR²¹, NR²¹R²², COOR²¹ or CONR²¹R²², lower haloalkyl, CN, F, ═O, SO₂NR¹⁹R²⁰, OR¹⁹, SR¹⁹, C(═O)OR¹⁹, C(═O)NR¹⁹R²⁰ or NR¹⁹R²⁰ in which R¹⁹ and R²⁰ are identical or different and are selected from hydrogen or lower alkyl optionally substituted with OR²¹, NR²¹R²², COOR²¹ or CONR²¹R²² in which R²¹ and R²² identical or different and are selected from hydrogen or lower alkyl, and, R¹⁹ and R²⁰, and/or, R²¹ and R²², together with the nitrogen atom to which they are linked, can form a 4- to 8-membered heterocyclic ring;
2 atoms of the cycle A, which are not adjacent, may be linked by a 2, 3 or 4 carbon atom chain which may be interrupted with 1 heteroatom chosen from O, S or N;
provided that:
not more than two of the groups A¹, A², A³, A⁴, A⁵ and A⁶ simultaneously represent a heteroatom;
the cycle A does not contain more than 2 carbon atoms in an sp₂ hybridization state;
when X is O, X₂ is not C—R¹ in which R¹ is
a thienyl substituted with CN or with CN and CH₃,
a phenyl substituted with CN, Cl, NO₂ or CN and F,
Br
F;
or their tautomeric forms, their racemic forms or their isomers and their pharmaceutically acceptable derivatives.

4. Use as claimed in claim 3, wherein the PDE7 inhibitor is 5′-(3-(Carboxy)propoxy)-8′-chlorospiro[cyclohexane-1,4′-quinazolin]-2′(1′H)-one, or a pharmaceutically acceptable salt or solvate thereof.

5. Use as claimed in claim 1 or 2, wherein the PDE7 inhibitor is a compound of formula (IV): 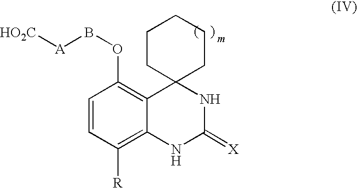
wherein:
m is 0, 1 or 2;
X is O, S or N—CN;
R is F, Cl or CN;
A is a C₃₋₆ cycloalkylene group optionally substituted with a C₁₋₂ alkyl group; and
B is a single bond or a C₁₋₂ alkylene group;
or a pharmaceutically acceptable salt, solvate or prodrug thereof.

6. Use as claimed in claim 5, wherein the PDE7 inhibitor is a compound selected from:
cis-3-[(8′-Chloro-2′-oxo-2′,3′-dihydro-1′H-spiro[cyclohexane-1,4′-quinazolin]-5′-yl)oxy]cyclobutanecarboxylic acid;
trans-3-[(8′-Chloro-2′-oxo-2′,3′-dihydro-1′H-spiro[cyclohexane-1,4′-quinazolin]-5′-yl)oxy]cyclobutanecarboxylic acid;
or a pharmaceutically acceptable salt, solvate or prodrug thereof.

7. Use as claimed in claim 1 or 2, wherein the PDE7 inhibitor is an antibody, an antibody ligand binding domain or a polynucleotide.

8. Use as claimed in any of claims 1 to 7 wherein the PDE7 inhibitor is used separately, sequentially or simultaneously in a combination combined with a second pharmacologically active compound.

9. Use as claimed in claim 8 wherein the second pharmacologically active compound of the combination is selected from;
an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine or pentazocine;
a nonsteroidal antiinflammatory drug (NSAID), e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac;
a barbiturate sedative, e.g. amobarbital, aprobarbital, butabarbital, butabital, mephobarbital, metharbital, methohexital, pentobarbital, phenobartital, secobarbital, talbutal, theamylal or thiopental;
a benzodiazepine having a sedative action, e.g. chlordiazepoxide, clorazepate, diazepam, flurazepam, lorazepam, oxazepam, temazepam or triazolam;
an H₁ antagonist having a sedative action, e.g. diphenhydramine, pyrilamine, promethazine, chlorpheniramine or chlorcyclizine;
a sedative such as glutethimide, meprobamate, methaqualone or dichloralphenazone;
a skeletal muscle relaxant, e.g. baclofen, carisoprodol, chlorzoxazone, cyclobenzaprine, methocarbamol or orphrenadine;
an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®, a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
an alpha-adrenergic, e.g. doxazosin, tamsulosin, clonidine, guanfacine, dexmetatomidine, modafinil, or 4-amino-6,7-dimethoxy-2-(5-methane-sulfonamido-1,2,3,4-tetrahydroisoquinol-2-yl)-5-(2-pyridyl)quinazoline;
a tricyclic antidepressant, e.g. desipramine, imipramine, amitriptyline or nortriptyline;
an anticonvulsant, e.g. carbamazepine, lamotrigine, topiratmate or valproate;
a tachykinin (NK) antagonist, particularly an NK-3, NK-2 or NK-1 antagonist, e.g. (αR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
a muscarinic antagonist, e.g oxybutynin, tolterodine, propiverine, tropsium chloride, darifenacin, solifenacin, temiverine and ipratropium;
a COX-2 selective inhibitor, e.g. celecoxib, rofecoxib, parecoxib, valdecoxib, deracoxib, etoricoxib, or lumiracoxib;
a coal-tar analgesic, in particular paracetamol;
a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
a vanilloid receptor agonist (e.g. resinferatoxin) or antagonist (e.g. capsazepine);
a beta-adrenergic such as propranolol;
a local anaesthetic such as mexiletine;
a corticosteroid such as dexamethasone;
a 5-HT receptor agonist or antagonist, particularly a 5-HT1B/1D agonist such as eletriptan, sumatriptan, naratriptan, zolmitriptan or rizatriptan;
a 5-HT₂A receptor antagonist such as R(+)-alpha-(2,3-dimethoxy-phenyl)-1-[2-(4-fluorophenylethyl)]-4-piperidinemethanol (MDL-100907);
a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
Tramadol®;
a PDEV inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′, 1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
a cannabinoid;
metabotropic glutamate subtype 1 receptor (mGluR1) antagonist;
a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
a noradrenaline (norepinephrine) reuptake inhibitor, such as maprotiline, lofepramine, mirtazepine, oxaprotiline, fezolamine, tomoxetine, mianserin, buproprion, buproprion metabolite hydroxybuproprion, nomifensine and viloxazine (Vivalan®), especially a selective noradrenaline reuptake inhibitor such as reboxetine, in particular (S,S)-reboxetine;
a dual serotonin-noradrenaline reuptake inhibitor, such as venlafaxine, venlafaxine metabolite O-desmethylvenlafaxine, clomipramine, clomipramine metabolite desmethylclomipramine, duloxetine, milnacipran and imipramine;
an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-5-chloro-3-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl)butyl]thio]-6-(trifluoromethyl)-3 pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, or guanidinoethyldisulfide;
an acetylcholinesterase inhibitor such as donepezil;
a prostaglandin E₂ subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(1S)-1-({[5-chloro-2-(3-fluorophenoxy)pyridin-3-yl]carbonyl}amino)ethyl]benzoic acid;
a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870,
a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl), 1,4-benzoquinone (CV-6504);
a sodium channel blocker, such as lidocaine;
a 5-HT3 antagonist, such as ondansetron;
and the pharmaceutically acceptable salts and solvates thereof.

10. A method for the treatment of neuropathic pain, in a mammalian subject, which comprises administering to said subject a therapeutically effective amount of an inhibitor of PDE7.

11. A method of treatment as claimed in claim 10 wherein the PDE7 inhibitor is a compound as defined in any one of claims 3 to 7 or is provided in a combination as defined in claims 8 to 9.
